(19) United States
(12) Patent Application Publication
(10) Pub. No.: US 2010/0173381 A1
(43) Pub. Date: Jul. 8, 2010

(54) CRYSTAL STRUCTURES OF HIV-1 PROTEASE INHIBITORS BOUND TO HIV-1 PROTEASE

(75) Inventors: Celia A. SCHIFFER, Shrewsbury, MA (US); Madhavi NALAM, Worcester, MA (US); Inge DIERYNCK, Berchem (BE); Annick Ann PEETERS, Wilrijk (BE); Tim Hugo JONKERS, Edegem (BE)

Correspondence Address: FISH & RICHARDSON PC, P.O. BOX 1022, MINNEAPOLIS, MN, 55440-1022, US

(73) Assignees: UNIVERSITY OF MASSACHUSETTS, Boston, MA (US); TIBOTEC PHARMACEUTICALS LTD, Co. Cork (IE)

(21) Appl. No.: 12/519,748

(22) PCT Filed: Dec. 18, 2007
(86) PCT No.: PCT/US2007/087990

§ 371 Date: Jan. 4, 2010

Related U.S. Application Data
(60) Provisional application No. 60/875,461, filed on Dec. 18, 2006.

(51) Int. Cl. C12N 9/96; G06G 7/48

(52) U.S. Cl. 435/188; 703/11

(57) ABSTRACT

Described herein are methods for rational design of inhibitors of HIV-1 protease, and crystal structures of HIV-1 protease inhibitors bound to HIV-1 protease.

### CROSS REFERENCE TO RELATED APPLICATION

This application claims priority from U.S. Provisional Patent Application No. 60/875,461, filed on Dec. 18, 2006, the contents of which are incorporated herein by reference in its entirety

### TECHNICAL FIELD

This invention relates to human immunodeficiency virus (HIV)-1 protease/inhibitor complexes, crystals of HIV-1 protease/inhibitor complexes, and related methods and software systems.

### BACKGROUND

Human immunodeficiency virus type 1 (HIV-1) protease plays an essential role in the viral life cycle by cleaving Gag and Gag-Pol polyproteins into structural and functional proteins necessary for viral assembly and maturation (Debouck, AIDS Res Hum Retroviruses 8, 153-164, 1992). Therefore HIV-1 protease is a prime target of drugs developed to control HIV/AIDS with nine protease-inhibitor drugs approved for clinical use since 1995 by the U.S. Food and Drug Administration. The nine protease inhibitors are saquinavir (SQV), indinavir (IDV), ritonavir (RTV), nelfinavir (NFV), amprenavir (APV), lopinavir (LPV), atazanavir (ATV), tipranavir (TPV) and darunavir (DRV/TMC114). All of these drugs are competitive inhibitors that bind in the active site of HIV-1 protease, and all these inhibitors, except for TPV, are peptidomimetics, i.e., they have a common hydroxyethylene or hydroxyethylamine core element instead of a peptide bond (Randolph and DeGoey, Curr Top Med Chem 4(10), 1079-1095, 2004). These core elements act as noncleavable peptide isosteres to mimic the transition state formed by the HIV-1 protease substrates during cleavage (Randolph and DeGoey, 2004, supra). The clinical pharmacokinetics and potency of these inhibitors were maximized by structure-based design.

Complexes between peptidomimetic inhibitors and HIV-1 protease are characterized by a noticeable structural feature, a conserved water molecule that mediates contacts between the P2/P1′ carbonyl oxygen atoms of the inhibitors and the amide groups of Ile50/Ile50′ of the enzyme (Wlodawer et al., Annu Rev Biochem 62, 543-585, 1993; Appelt, Perspect Drug Discovery Des 1, 23-48, 1993). Replacing this conserved water was proposed as a way of making highly specific protease inhibitors (Swain et al., Proc Natl Acad Sci USA 87, 8805-8809, 1990). This approach was used to design compounds with a 7-membered cyclic urea ring as the starting pharmacophore (Lam et al., J Med Chem 39, 3514-3525, 1996). The crystal structure of this cyclic urea compound in complex with HIV-1 protease showed that the urea oxygen replaces the role of the conserved water. One of these cyclic urea inhibitors, DMP-450, was shown to have excellent inhibitory properties, was highly potent against the virus in cell cultures, and was orally bioavailable in humans. DMP-450 showed promising results until Phase III trials, but its development was discontinued due to safety concerns (Rusconi et al., Therapy 3(1), 79-88, 2006). TPV is another protease inhibitor in which the conserved water is replaced by the lactone oxygen atom of the inhibitor's dihydropyrone ring (Turner et al., J Med Chem 41, 3467-3476, 1998). TPV was the first nonpeptidic compound among the currently marketed protease inhibitors (Flexner et al., Nat Rev Drug Discov 4, 955-956, 2005).

The development of protease inhibitors has improved the lives of AIDS patients and contributed to the success of highly active anti-retroviral therapy (HAART). However, the rapid emergence of resistance to these protease inhibitors has become a major issue. This problem has generated a pressing need to improve current drugs in terms of greater antiretroviral potency, bioavailability, toxicity, and higher activity towards drug-resistant mutant viruses. These goals are being targeted by the development of many second-generation protease inhibitors.

One way of developing new drugs is to modify the substituents of existing protease inhibitors or to design totally new molecular cores. Recently lysine sulfonamides were developed as novel HIV-1 protease inhibitors (Stranix et al., Bioorg Med Chem Lett 13, 4289-4292, 2003). One of these lysine sulfonamides, PL-100, is highly potent against drug-resistant proteases and exhibits a favorable cross-resistance profile against the marketed protease inhibitors (Wu et al., 15th International HIV Drug Resistance Workshop, Jun. 13-17, 2006 Sitges, Spain; Abstract published in Antiviral Therapy 11:S152 (2006); poster available at ambrilia.com/en/products/hiv-aids-pPPL-100-references.php). PPL-100, a prodrug of PL-100, is in Phase I human clinical trials with promising results thus far (Wu et al., 2006, supra). Another lysine sulfonamide inhibitor with a chemical structure similar to that of PL-100 is P867883 (see FIGS. 1A-B).

### SUMMARY

The present invention is based, at least in part, on the elucidation of the crystal structures of several novel inhibitors, including P867883, in complex with HIV-1 protease. These crystal structures can be used in rational drug design methods. In particular, P867883 binds to the protease in a novel mode by replacing the conserved water, and thus provides a completely new structural paradigm for inhibitor design, which may yield inhibitors that are less susceptible to the development of drug-resistant viruses, e.g., P867883 analogs as described herein.

In one aspect, the invention features a crystallized HIV-1 protease/inhibitor complex that includes an HIV-1 protease and an inhibitor described herein, e.g., P867883. As used herein, “an HIV-1 protease” is a dimer formed by two identical HIV-1 protease polypeptides. The amino acids of the two polypeptides are differentiated herein by the use of the notation “prime” 0 on the amino acids of one of the polypeptides. Thus, Asp50 and Asp50′ refer to amino acid 50 in each of the two polypeptides.

In another aspect, the invention features a composition that includes a crystal. The crystal includes an HIV-1 protease and an inhibitor described herein, e.g., P867883.

In another aspect, the invention features a method that includes using a three-dimensional model of a complex that includes an HIV-1 protease. In some embodiments, the complex includes a fragment of the protease as defined by structural coordinates of amino acids sufficient to define a binding pocket. The structural coordinates can be as shown in table 2, or a homolog thereof that has a root mean square deviation of not more than 1.5 Angstroms from the backbone atoms of the amino acids as shown in table 2. The protease can be free (unbound) or bound to an inhibitor, e.g., P867883. The three-dimensional model can be used to select or design an inhibitor that binds the HIV-1 protease, with specific binding features as described herein, e.g., the formation of hydrogen bonds with the protease. In some embodiments, employing the three-dimensional structural model to design or select a potential inhibitor includes providing a three-dimensional model of the potential inhibitor, employing computational means to perform a fitting operation between the model of the potential inhibitor and the model of the HIV-1 protease active site to provide an energy minimized configuration of the potential inhibitor in the active site, and evaluating the results of the fitting operation to design or select a potential inhibitor that has the specified interactions with the HIV-1 protease.

As used herein, a “hydrogen bond” is an interaction between a proton acceptor and a proton donor that forms when the proton-acceptor distance is less than 2.5 Angstroms and the angle defined by the donor-hydrogen-acceptor atoms lies between 90 and 180 degrees (see, e.g., Baker and Hubbard, Prog. Biophys. Molec. Biol. 44:97-179 (1984)).

In a further aspect, the invention features methods that include using a three-dimensional model of an HIV-1 protease to select or design an inhibitor that binds the HIV-1 protease.

In some embodiments, the methods include the use of a three-dimensional structural model that includes at least the atomic coordinates of the atoms of HIV-1 protease amino acids 24-30, 24′-30′, 47-53, 47′-53′, 84 and 84′, and optionally amino acids 82 and 82′, according to Table 2± a root mean square deviation from the backbone atoms of said amino acids of not more than 1.5 Å. In some embodiments, a three-dimensional structural model is a computer model.

In another aspect, the invention features methods that include selecting an inhibitor by performing rational drug design with a three-dimensional structure of a crystalline complex.

In some embodiments, the potential inhibitors identified, designed or selected by a method described herein form at least one hydrogen bond with the backbone, e.g., the backbone nitrogen atoms, of amino acids 50 and 50′ of the HIV-1 protease via a sulfonyl or selenonyl group without an intervening water molecule. In some embodiments, the potential inhibitors form at least one hydrogen bond with the backbone, e.g., the backbone nitrogen atoms, of amino acids 50 and 50′ of the HIV-1 protease via an acyclic group without an intervening water molecule.

A sulfonyl group is an organic radical or functional group obtained from a sulfonic acid by the removal of the hydroxyl group. Sulfonyl groups can be written as having the general formula R—S(═O)₂—R′, where there are two double bonds between the sulfur and oxygen. A selenonyl group is a selenium version of a thioketone group, and can be written as R—Se(═O)₂—R′, where there are two double bonds between the selenium and oxygen.

As used herein, an “intervening water molecule” is a water molecule that forms hydrogen bonds with both the potential inhibitor and the specified amino acid, linking the two together. For example, the conserved water molecule shown in FIG. 3 is an intervening water molecule that binds to both the inhibitor Amprenavir and the backbone of amino acids 50 and 50′ of the flap regions of the HIV-1 protease.

In some embodiments, the potential inhibitors further form at least one hydrogen bond with the conserved side chain atoms, e.g., oxygen atoms, of at least one of amino acids Asp25 and Asp25′ of the HIV-1 protease, e.g., a hydrogen bond with the side chain that is formed by a primary hydroxyl, thiol, or amino group on the potential inhibitor. In some embodiments, the hydrogen bond is a bifurcated hydrogen bond. In some embodiments, the potential inhibitor does not hydrogen bond with any atoms of amino acid 27 of the HIV-1 protease. In some embodiments, the potential inhibitor forms at least one hydrogen bond with a backbone atom, e.g., a nitrogen atom, of one or both of amino acids 48 or 28 of the HIV-1 protease, and/or forms at least one hydrogen bond with atoms of the conserved side chain, e.g., the oxygens atoms, of amino acid Asp29 of the HIV-1 protease

In some embodiments, the potential inhibitors identified, designed or selected by a method described herein have the following interactions with the HIV-1 protease:
(a) hydrogen bonding with the backbone, e.g., the backbone nitrogen atoms, of amino acids 50 and 50′ of the HIV-1 protease, without an intervening water molecule; and
(b) direct hydrogen bonding with the conserved side chain, e.g., the side chain oxygens, of amino acid Asp25 and Asp25′ of the HIV-1 protease;

and two or more of the following:
(c) no hydrogen bonding interaction with any atoms of amino acid 27 of the HIV-1 protease;
(d) direct hydrogen bonding with the backbone, e.g., the backbone nitrogen atoms, of amino acids 48 and 28 of the HIV-1 protease; and
(e) direct hydrogen bonding with the conserved side chain of amino acid Asp29, e.g., the side chain oxygen atoms, of the HIV-1 protease.
   In some embodiments, the potential inhibitors have all of interactions (a)-(e) with the HIV-1 protease. In some embodiments, the potential inhibitors additionally have one of the following interactions with the HIV-1 protease:
(f) direct hydrogen bonding with the backbone, side chains, or both, of one or more of amino acids 29′, 30′, and 48′ of the HIV-1 protease; or
(g) indirect (e.g., via an intervening water molecule) hydrogen bonding with the backbone, side chains, or both, of one or more of amino acids 29′, 30′, and 48′ of the HIV-1 protease.

In some embodiments, the designed inhibitor is then synthesized or otherwise obtained, and contacted with an HIV-1 protease, and the ability of the inhibitor to bind and/or inhibit the HIV-1 protease is detected.

In yet another aspect, the invention features a method that includes contacting an HIV-1 protease with an inhibitor to form a composition and crystallizing the composition to form a crystalline complex where the inhibitor is bound to the HIV-1 protease. The crystalline complex can diffract X-rays to a resolution of at least about 3.5 Å, e.g., 2 Å. The inhibitor is an inhibitor described herein, e.g., P867883.

In another aspect, the invention features a software system that includes instructions for causing a computer system to accept information relating to the structure of an HIV-1 protease bound to an inhibitor, accept information relating to a candidate inhibitor, and determine binding characteristics of the candidate inhibitor to the HIV-1 protease. Determination of the binding characteristics is based on the information relating to the structure of the HIV-1 protease bound to the inhibitor and the information relating to the candidate inhibitor. The inhibitor is an inhibitor described herein, e.g., P867883.

In another aspect, the invention features a computer program on a computer readable medium on which is stored a plurality of instructions. When the instructions are executed by one or more processors, the processors accept information relating to the structure of a complex that includes an HIV-1 protease bound to an inhibitor. The processors further accept information relating to a candidate inhibitor and determine binding characteristics of the candidate inhibitor to the HIV-1 protease. Determination of the binding characteristics is based on the information related to the structure of the HIV-1 protease and the information related to the candidate inhibitor.

In a further aspect, the invention features a method that includes accepting information relating to the structure of a complex including an HIV-1 protease bound to an inhibitor and modeling the binding characteristics of the HIV-1 protease with a candidate inhibitor. Such a method is implemented by a software system.

In another aspect, the invention features a computer program on a computer readable medium on which is stored a plurality of instructions. When the instructions are executed by one or more processors, the processors accept information relating to a structure of a complex that includes an HIV-1 protease bound to an inhibitor. The processors further model the binding characteristics of the HIV-1 protease with a candidate inhibitor.

In an additional aspect, the invention features a software system that includes instructions for causing a computer system to accept information relating to a structure of a complex that includes an HIV-1 protease bound to an inhibitor. The instructions also cause a computer system to model the binding characteristics of the HIV-1 protease with a candidate inhibitor.

In another aspect, the invention features a method of modulating HIV-1 protease activity in a subject. The method includes using rational drug design to select an inhibitor that is capable of modulating HIV-1 protease activity, and administering a therapeutically effective amount of the inhibitor to the subject.

In another aspect, the invention features a method of treating a subject having a condition associated with HIV-1 protease activity. The method includes using rational drug design to select an inhibitor that is capable of affecting HIV-1 protease activity and administering a therapeutically effective amount of the inhibitor to a subject in need of such an inhibitor.

In another aspect, the invention features a method of prophylactically treating a subject susceptible to a condition associated with HIV-1 protease activity. The method includes determining that the subject is susceptible to the condition associated with the activity, using rational drug design to select an inhibitor that is capable of reducing HIV-1 protease activity, and administering a therapeutically effective amount of the inhibitor to the subject.

The following abbreviations are used throughout the application:

| | Singe Letter | Three Letter |
|---|---|---|
| Amino Acid | Abbreviation | Abbreviation |
| Alanine | A | Ala |
| Threonine | T | Thr |
| Valine | V | Val |
| Cysteine | C | Cys |
| Leucine | L | Leu |
| Tyrosine | Y | Tyr |
| Isoleucine | I | Ile |
| Asparagine | N | Asn |
| Proline | P | Pro |
| Glutamine | Q | Gln |
| Phenylalanine | F | Phe |
| Aspartic Acid | D | Asp |
| Tryptophan | W | Trp |
| Glutamic Acid | E | Glu |
| Methionine | M | Met |
| Lysine | K | Lys |
| Glycine | G | Gly |
| Arginine | R | Arg |
| Serine | S | Ser |
| Histidine | H | His |

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIGS. 1A-D are the chemical structures of P867883 (1A), PL-100(1B), Amprenavir (APV), 1C), and Tipranavir (TPV, 1D).

FIG. 2A is a ribbon diagram of HIV-1 protease dimer (in dark and medium grey) bound to P867883 (in light grey). The oxygen and nitrogen atoms are shown in black.

FIG. 2B is a model showing hydrogen bonding interactions between the HIV-1 protease and P867883 (shown in gray). The nitrogen and oxygen atoms are shown in black and white respectively. Notice that the inhibitor makes hydrogen bonds directly to the flaps, unlike in other peptidomimetic inhibitors.

FIG. 2C is a packing diagram of the HIV-1 protease with P867883. The nitrogen and oxygen atoms are shown in black and white respectively.

FIG. 3 is a model showing hydrogen bonding interactions between the HIV-1 protease and Amprenavir, a peptidomimetic inhibitor. A water molecule mediates hydrogen bonding between the flaps of the protease (at the top of the drawing) and the inhibitor (in the center).

FIG. 4 is a model showing hydrogen bonding interactions between the HIV-1 protease and Tipranavir, a non-peptidomimetic inhibitor. Direct hydrogen bonding occurs between the flaps of the protease (at the top of the drawing) and the inhibitor (in the center).

### DETAILED DESCRIPTION

In general, the present invention relates to HIV-1 protease/inhibitor complexes, crystals of HIV-1 protease/inhibitor complexes, and related methods and software systems, including methods using the crystal structures of HIV-1 protease/inhibitor complexes for designing or identifying other inhibitors of HIV-1 protease.

HIV-1 Protease Polypeptides

As noted above, an HIV-1 protease is made up of two identical HIV-1 protease polypeptides. An exemplary HIV-1 protease polypeptide sequence used in the methods and structures described herein is as follows:

(SEQ ID NO: 1)

PQITLWKRPLVTIRIGGQLKEALLDTGADDTVLEEMNLPGKWKPKMIGGI
GGFIKVRQYDQIPIEICGHKAIGTVLVGPTPVNIIGRNLLTQIGCTLNF

The methods can also be carried out using other variants of HIV-1 protease polypeptide, e.g., as found in the HIV reverse transcriptase and protease sequence database, an on-line relational database that catalogues evolutionary and drug-related sequence variation in the human immunodeficiency virus (HIV) reverse transcriptase (RT) and protease enzymes, the molecular targets of antiretroviral therapy (hivdb.stanford.edu, described in Rhee et al., Nuc. Acids Res. 31(1):298-303 (2003)).

For example, other HIV-1 protease polypeptide sequences that can be used include:

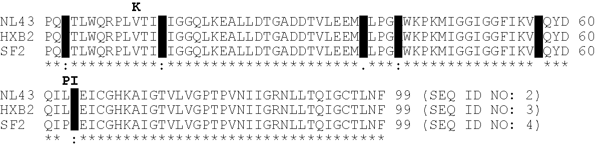

In some embodiments, the HIV-1 protease polypeptide sequence is SF2. The SF2 shown above (SEQ ID NO:4) is the original, wild type sequence. In some embodiments, the sequence includes modifications; for example, the protease polypeptide sequence that was used in the examples set forth herein describing the crystallization of the protease/P867883 complex includes Gln7Lys and Val64Ile mutations to the SF2 sequence above. In some embodiments, the HIV-1 protease polypeptide sequence is as shown in SEQ ID NO:1.

HIV-1 Protease/Inhibitor P867883 Complex Compositions

The HIV-1 protease polypeptides can be produced by any known method, including synthetic methods, such as solid phase, liquid phase and combination solid phase/liquid phase syntheses; recombinant DNA methods, including cDNA cloning, optionally combined with site directed mutagenesis; and/or purification of the natural products, optionally combined with enzymatic cleavage methods to produce fragments of naturally occurring HIV-1 protease polypeptides. The P867883 inhibitor can also be produced by any known method, e.g., as described in pending application EP 06.114.672.6, filed 30 May 2006, and Nalam et al., J. Virol., 81(17):9512-9518 (2007). According to a preferred embodiment, the compositions described herein are crystallizable.

HIV-1 Protease/Inhibitor P867883 Complex Structures

Advantageously, the crystallizable compositions provided herein are amenable to x-ray crystallography. Thus, provided herein is the three-dimensional structure of an HIV-1 protease/inhibitor P867883 complex, at 2.5 Angstrom resolution or better. In some embodiments, the three-dimensional structure of the HIV-1 protease/inhibitor P867883 complex described herein is defined by a set of structural coordinates as set forth in table 2. The term “structural coordinates” refers to Cartesian coordinates derived from mathematical equations related to the patterns obtained on diffraction of a monochromatic beam of X-rays by the atoms (scattering centers) of an HIV-1 protease/inhibitor P867883 complex in crystal form. The diffraction data are used to calculate an electron density map of the repeating unit of the crystal. The electron density maps are then used to establish the positions of the individual atoms of the HIV-1 protease/inhibitor P867883 complex.

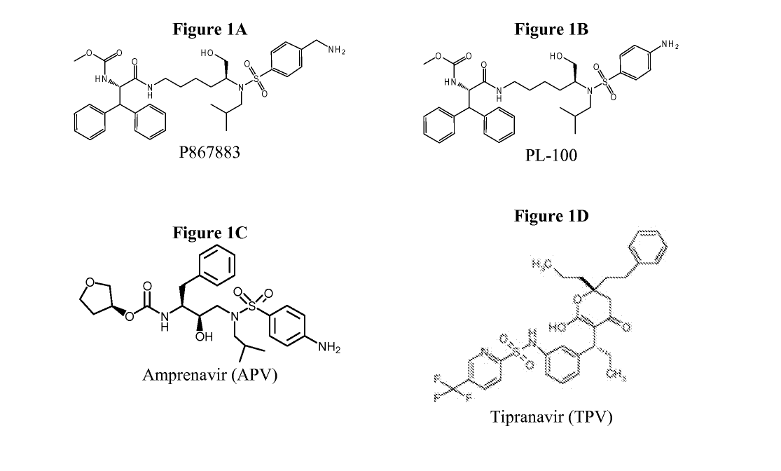

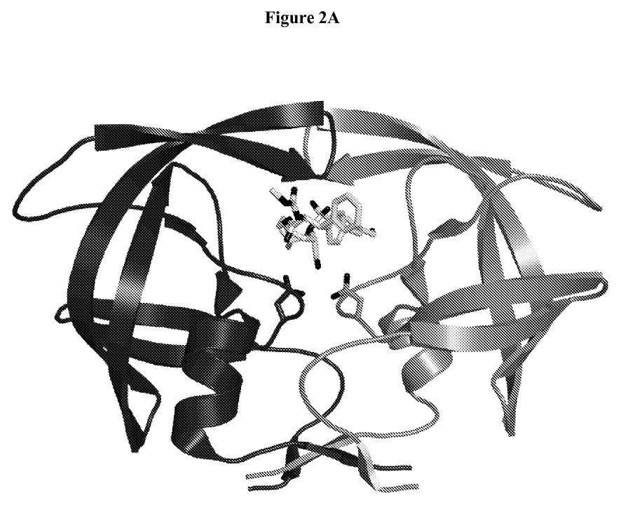

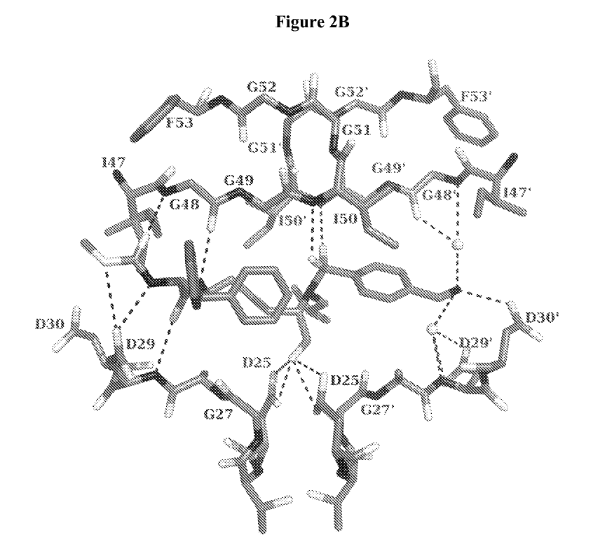

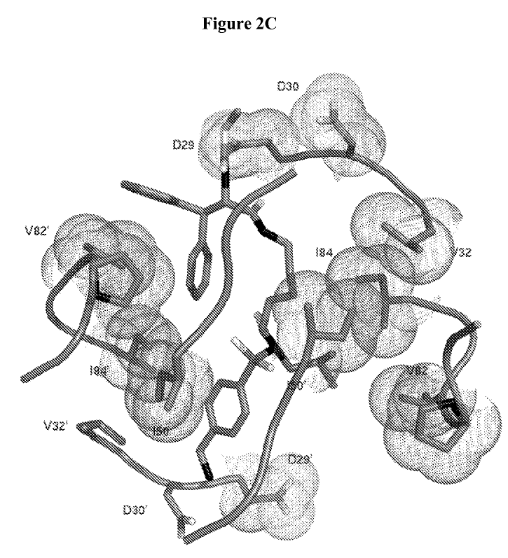

By solving the structure of a complex between HIV-1 protease and the inhibitor P867883, the present inventors have shown that this inhibitor binds in a novel way to the active site compared to other protease inhibitors in clinical use. P867883 is a nonpeptidic, competitive inhibitor (see FIG. 1A). As demonstrated herein, P867883 forms hydrogen bonds mostly to protease main-chain atoms or conserved side-chain atoms (see FIGS. 2A-2C). This bonding pattern is a favorable situation for the inhibitor since the pattern is less likely to be affected by drug-resistant mutations in protease residues of the active site.

The structure of P867883 is similar to PL-100 (see FIG. 1B; also see U.S. Pat. No. 6,632,816; PL-100 is available from Ambrilia Biopharma Inc.). The only P867883 hydrogen-bonding interaction with a nonconserved residue side chain is from the P2′ benzyl amine group to Asp30′. This interaction may not be present in PL-100 since it has a P2′ phenyl amine group, one methylene group less than the benzyl amine group of P867883. PPL-100 is in Phase I clinical trials (Wu et al., 2006, supra). Although the structure of the PL-100-protease complex has not yet been published, its hydrogen bonding patterns are likely to be similar to those of the P867883-protease complex, except for bonds made by the amine group at P2′ where the benzyl amine is replaced by a phenyl amine in PL-100. PL-100 may form water-mediated hydrogen bonds similar to P867883 (See FIG. 2B).

Insights into the clinical significance of PL-100's hydrogen-bonding pattern can be gained by examining PL-100's pattern of selecting for protease mutations in vitro. When PL-100 was subjected to an in vitro test of its ability to select for protease mutants conferring resistance (see Wu et al., 2006, supra), a novel selection pattern was found of four mutations (K45R, M46I, T80I, and P81S). Single-, double- or triple-viral mutants did not show resistance to PL-100, and only mild resistance was observed with the quadruple-viral mutant (Wu et al., 2006, supra). PL-100 also shows a favorable cross-resistance profile to the clinical protease inhibitors APV, LPV, ATV, SQV, IDV and NFV (Wu et al., 2006, supra). Since these inhibitors select for signature protease mutations in the active site, they do not affect the hydrogen-bonding pattern of PL-100. Thus, the experimental observations for PL-100 are consistent with predictions of its structure based on the crystal structure of P867883 in complex with HIV-1 protease.

P867883, and hence PL-100, has properties that contribute to it binding differently from other protease inhibitors to HIV-1 protease. P867883 has a primary OH group that interacts with the two catalytic aspartic acids, Asp25 and Asp25′, whereas all other peptidomimetic protease inhibitors have a secondary OH group that interacts with Asp25 and Asp25′. The hydrogen bonding between this primary OH group in P867883 and Asp25 and Asp25′ is facilitated by the methylene group that connects the hydroxyl group and the inhibitor core; the methylene group pushes the entire inhibitor towards the protease flaps. This displacement in turn brings the two oxygen atoms of the inhibitor's sulfone group within hydrogen-bonding distance of Ile50 and Ile50′.

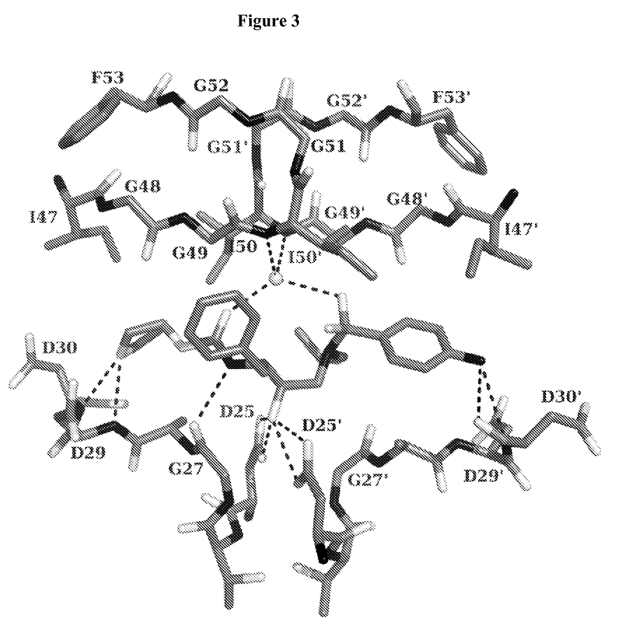

All other protease inhibitors lack the methylene group, accounting in part for the different binding of P867883. For instance, in APV, a peptidomimetic inhibitor whose P1′ and P2′ are similar to those of P867883 (compare FIGS. 1A and 1C), the secondary hydroxyl group forms hydrogen bonds with Asp25 and Asp25′ as in P867883 (FIG. 3). However, APV is closer than P867883 to the bottom of the active site cavity and further from the flap region of the protease. APV cannot directly form hydrogen bonds with the flaps and hence requires a water molecule to mediate its interactions with the protease flaps. This need for a water molecule is true for all other peptidomimetic inhibitors. The presence of a primary hydroxyl group instead of a secondary hydroxyl group might be a primary reason for the novel binding of P867883 to the protease, resulting in the absence of the conserved water molecule in the crystal structure.

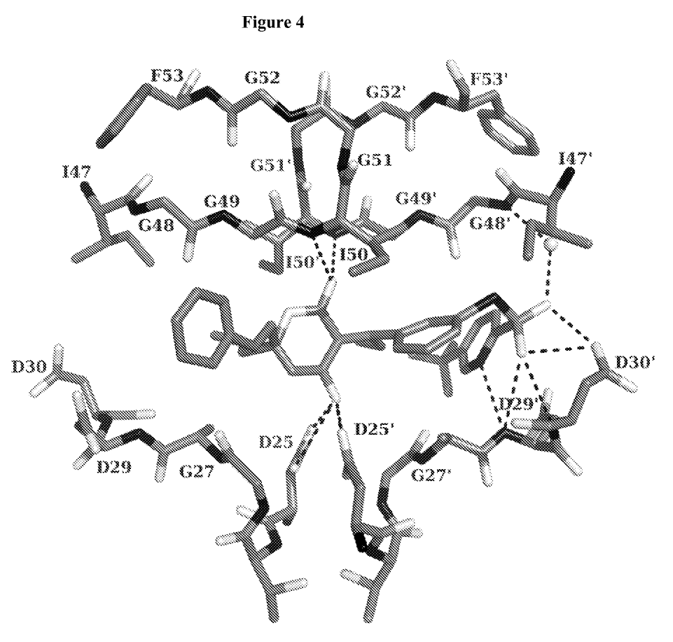

TPV and P867883 are non-peptidomimetic protease inhibitors with a different binding mode than previously described for other protease inhibitors (compare FIGS. 1A and 1C, and see FIG. 4). P867883 and TPV have two features that distinguish them from other protease inhibitors. First, these two inhibitors do not form hydrogen bonds with Gly27 in the floor of the protease active site. This interaction with Gly27 is conserved in all other known protease-substrate and protease-inhibitor complexes. Second, both inhibitors form hydrogen bonds directly with the protease flaps, without the mediation of a water molecule, although each inhibitor binds differently. In TPV, the lactone oxygen atom of the dihydropyrone ring forms hydrogen bonds with the amide nitrogen atoms of the flap residues, analogous to how water molecules in peptidomimetic inhibitors interact with flap residues. In contrast, in P867883, two oxygen atoms from the sulfone group form two hydrogen bonds with the flap residues. This kind of interaction distinguishes the binding of P867883 not only from that of TPV, but also from that of all known crystal structures of protease-inhibitor complexes.

Structural Models

As described herein, X-ray crystallography can be used to obtain structural coordinates of a complex of HIV-1 protease bound to an inhibitor. However, such structural coordinates can be obtained using other techniques, including NMR and other techniques known in the art. Additional structural information can be obtained from spectral techniques (e.g., optical rotary dispersion (ORD), circular dichroism (CD)), homology modeling, and computational methods (e.g., computational methods that can include data from molecular mechanics, computational methods that include data from dynamics assays).

Various software programs allow for the graphical representation of a set of structural coordinates, e.g., the coordinates provided herein, to obtain a structural model that represents a complex of HIV-1 protease bound to an inhibitor, or a portion of one of these complexes. In general, such a representation should accurately reflect (relatively and/or absolutely) structural coordinates, or information derived from structural coordinates, such as distances or angles between features. In some embodiments, the representation is a two-dimensional figure, such as a stereoscopic two-dimensional figure. In certain embodiments, the representation is an interactive two-dimensional display, such as an interactive stereoscopic two-dimensional display. An interactive two-dimensional display can be, for example, a computer display that can be rotated to show different faces of a polypeptide, a fragment of a polypeptide, a complex and/or a fragment of a complex.

In some embodiments, the representation is a three-dimensional representation. As an example, a three-dimensional model can be a physical model of a molecular structure (e.g., a ball-and-stick model). As another example, a three dimensional representation can be a graphical representation of a molecular structure (e.g., a drawing or a figure presented on a computer display). A two-dimensional graphical representation (e.g., a drawing) can correspond to a three-dimensional representation when the two-dimensional representation reflects three-dimensional information, for example, through the use of perspective, shading, or the obstruction of features more distant from the viewer by features closer to the viewer.

In some embodiments, a representation can be modeled at more than one level. As an example, when the three-dimensional representation includes a polypeptide, such as a complex of HIV-1 protease bound to an inhibitor, the polypeptide can be represented at one or more different levels of structure, such as primary (amino acid sequence), secondary (e.g., α-helices and β-sheets), tertiary (overall fold), and quaternary (oligomerization state) structure. A representation can include different levels of detail. For example, the representation can include the relative locations of secondary structural features of a protein without specifying the positions of atoms. A more detailed representation could, for example, include the positions of atoms.

In some embodiments, a representation can include information in addition to the structural coordinates of the atoms in a complex of HIV-1 protease bound to an inhibitor. For example, a representation can provide information regarding the shape of a solvent accessible surface, the van der Waals radii of the atoms of the model, and the van der Waals radius of a solvent (e.g., water). Other features that can be derived from a representation include, for example, electrostatic potential, the location of voids or pockets within a macromolecular structure, and the location of hydrogen bonds and salt bridges.

In some embodiments, X-ray diffraction data can be used to construct an electron density map of a complex of HIV-1 protease bound to an inhibitor or a fragment thereof, and the electron density map can be used to derive a representation (e.g., a two dimensional representation, a three dimensional representation) of HIV-1 protease bound to an inhibitor, or a portion thereof. Creation of an electron density map typically involves using information regarding the phase of the X-ray scatter. Phase information can be extracted, for example, either from the diffraction data or from supplementing diffraction experiments to complete the construction of the electron density map. Methods for calculating phase from X-ray diffraction data include, for example, multiwavelength anomalous dispersion (MAD), multiple isomorphous replacement (MIR), multiple isomorphous replacement with anomalous scattering (MIRAS), single isomorphous replacement with anomalous scattering (SIRAS), reciprocal space solvent flattening, molecular replacement, or any combination thereof.

Upon determination of the phase, an electron density map can be constructed. The electron density map can be used to derive a representation of the complex or a fragment thereof, e.g., by aligning a three-dimensional model of a previously solved HIV-1 protease (e.g., pdb reference no. 1F7A) or a previously known complex (e.g., a complex containing a HIV-1 protease bound to an inhibitor) with the electron density map. For example, the electron density map corresponding to a HIV-1 protease/inhibitor complex can be aligned with the electron density map corresponding to HIV-1 protease complexed to another compound, such as another inhibitor, or to a mutant or variant HIV-1 protease.

The alignment process results in a comparative model that shows the degree to which the calculated electron density map varies from the model of the previously known polypeptide or the previously known complex. The comparative model is then refined over one or more cycles (e.g., two cycles, three cycles, four cycles, five cycles, six cycles, seven cycles, eight cycles, nine cycles, 10 cycles) to generate a better fit with the electron density map. A software program such as CNS (Brunger et al., Acta Crystallogr. D54:905-921, 1998) can be used to refine the model. The quality of fit in the comparative model can be measured by, for example, an R_{work} or R_{free} value. A smaller value of R_{work} or R_{free} generally indicates a better fit. Misalignments in the comparative model can be adjusted to provide a modified comparative model and a lower R_{work} or R_{free} value. The adjustments can be based on information (e.g., sequence information) relating to, e.g., HIV-1 protease, alone or bound to another inhibitor.

As an example, in embodiments in which a model of a previously known complex of a HIV-1 protease bound to an inhibitor is used, an adjustment can include replacing the inhibitor in the previously known complex with a test or candidate inhibitor as described herein. As another example, in certain embodiments, an adjustment can include replacing an amino acid in the HIV-1 protease used previously with the amino acid in the corresponding site of a mutant or variant HIV-1 protease. When adjustments to the modified comparative model satisfy a best fit to the electron density map, the resulting model is that which is determined to describe the polypeptide or complex from which the X-ray data was derived (e.g., an HIV-1 protease inhibitor complex). Methods of such processes are disclosed, for example, in Carter and Sweet, eds., “Macromolecular Crystallography” in Methods in Enzymology, Vol. 277, Part B, New York: Academic Press, 1997, and articles therein, e.g., Jones and Kjeldgaard, “Electron-Density Map Interpretation,” p. 173, and Kleywegt and Jones, “Model Building and Refinement Practice,” p. 208.

Those of skill in the art will understand that a set of structure coordinates for a complex as described herein is a relative set of points that define a shape in three dimensions. Thus, it is possible that an entirely different set of coordinates could define a similar or identical shape. Moreover, slight variations in the individual coordinates will have little effect on overall shape.

For example, variations in coordinates may be generated because of mathematical manipulations of the structure coordinates. For example, the structure coordinates set forth in Table 2 could be manipulated by crystallographic permutations of the structure coordinates, fractionalization of the structure coordinates, integer additions or subtractions to sets of the structure coordinates, inversion of the structure coordinates or any combination of the above.

Alternatively, modifications in the crystal structure due to mutations, additions, substitutions, and/or deletions of amino acids, or other changes in any of the components that make up the crystal, e.g., substitution of different HIV-1 protease polypeptides, could also account for variations in structure coordinates. If such variations are within an acceptable standard error as compared to the original coordinates, the resulting three-dimensional shape is considered to be the same.

Various computational analyses can be used to determine whether a molecule or molecular complex or a portion thereof is sufficiently similar to all or parts of the HIV-1 protease/inhibitor complex described herein as to be considered the same. Such analyses may be carried out in current software applications, such as the Molecular Similarity application of QUANTA (Molecular Simulations Inc., San Diego, Calif.) version 4.1, and as described in the accompanying User's Guide.

For the purpose of the methods described herein, any molecule or molecular complex that has a root mean square deviation of conserved residue backbone atoms (N, Cα, C, or O) of less than 1.5 Angstrom when superimposed on the relevant backbone atoms described by structure coordinates listed in Table 2 are considered identical. In some embodiments, the root mean square deviation is less than 1.0 Angstrom.

The term “root mean square deviation” means the square root of the arithmetic mean of the squares of the deviations from the mean, and expresses the deviation or variation from a trend or object. For purposes of the present application, the “root mean square deviation” defines the variation in the backbone of a protein or protein complex from the relevant portion of the backbone of the HIV-1 protease portion of the complex as defined by the structure coordinates described herein.

A machine, such as a computer, can be programmed in memory with the structural coordinates of a complex of the HIV-1 protease or the HIV-1 protease bound to an inhibitor as described herein, together with a program capable of generating a graphical representation of the structural coordinates on a display connected to the machine. Alternatively or additionally, a software system can be designed and/or utilized to accept and store the structural coordinates. The software system can be capable of generating a graphical representation of the structural coordinates. The software system can also be capable of accessing external databases to identify compounds (e.g., polypeptides) with similar structural features as an inhibitor described herein, and/or to identify one or more candidate inhibitors with characteristics that may render the candidate inhibitor(s) likely to interact with HIV-1 protease in a manner similar to an inhibitor described herein, e.g., P867883. Thus, the structural coordinates of a HIV-1 protease polypeptide/P867883 complex and portions thereof can be stored in a machine-readable storage medium. Such data may be used for a variety of purposes, such as drug discovery and x-ray crystallographic analysis or protein crystal. Accordingly, also provided herein is a machine-readable data storage medium comprising a data storage material encoded with the structure coordinates set forth in Table 2.

Rational Design of Candidate HIV-Protease Inhibitors

HIV-1 resists efforts to find a cure that will eradicate the virus from infected individuals or to develop a vaccine. Until a safe and effective vaccine is developed against HIV-1, new drugs need to be developed to reach high plasma levels and to possibly overcome the cross-resistance among various inhibitors. One approach to overcoming cross-resistance is to design new inhibitors that not only bind tightly to mutant proteases but also bind in a mode different from existing inhibitors. Support for this approach comes from TPV, which has a different binding mode and has been shown to bind to drug-resistant mutants (Rusconi et al., Antimicrob Agents Chemother 44, 1328-32, 2000). The crystal structure of the P867883-protease complex presented herein provides a new direction for designing candidate protease inhibitors. In fact, PPL-100, which is similar to P867883, is currently in Phase-I clinical trials and showing promising results. Now, however, with the structure of P867883 in complex with HIV-1 protease it is possible to design other inhibitors that fill the active site cavity in a novel manner.

Novel inhibitors of HIV-1 protease can be identified or designed by a method that includes using a model of HIV-1 protease or a portion thereof (e.g., of the active site, as described herein, or a complex of HIV-1 protease bound to an inhibitor described herein or a portion of either one of these complexes.

In some embodiments, the representation can be of an analog polypeptide, e.g., a mutant or variant of HIV-1 protease, alone or in a complex with an inhibitor, e.g., an inhibitor described herein or known in the art. A candidate inhibitor that interacts with the representation can be designed or identified by performing computer fitting analysis of the candidate inhibitor with the representation. Examples of candidate inhibitors include peptides, peptidomimetics, and small organic or inorganic molecules. The interaction can be mediated by any of the forces noted herein, including, for example, hydrogen bonding, electrostatic forces, hydrophobic interactions, and van der Waals interactions.

As noted above, X-ray crystallography, NMR, or other methods can be used to obtain structural coordinates of a complex of HIV-1 protease bound to an inhibitor.

A machine having a memory containing structure data or a software system containing such data, as described herein, can aid in the rational design or selection of candidate HIV-1 protease inhibitors. For example, such a machine or software system can aid in the evaluation of the ability of a candidate inhibitor to associate with HIV-1 protease in a manner similar to an inhibitor described herein, e.g., P867883, or can aid in the modeling of compounds related by structural homology to P867883, e.g., structural analogs that are or may be candidate inhibitors.

The machine can produce a representation (e.g., a two dimensional representation, a three dimensional representation) of the active site of the HIV-1 protease or a complex of the HIV-1 protease or a portion thereof, e.g., the active site of the HIV-1 protease, alone or bound to an inhibitor. A software system, for example, can cause the machine to produce such information. The machine can include a machine-readable data storage medium including a data storage material encoded with machine-readable data. The machine-readable data can include structural coordinates of atoms of HIV-1 protease or a complex of HIV-1 protease bound to an inhibitor, or a portion thereof, e.g., the active site of the HIV-1 protease. Machine-readable storage media (e.g., data storage material) include, for example, conventional computer hard drives, floppy disks, DAT tape, CD-ROM, DVD, and other magnetic, magneto-optical, optical, and other media which may be adapted for use with a machine (e.g., a computer).

The machine can also have a working memory for storing instructions for processing the machine-readable data, as well as a central processing unit (CPU) coupled to the working memory and to the machine-readable data storage medium for the purpose of processing the machine-readable data into the desired three-dimensional representation. A display can be connected to the CPU so that the three-dimensional representation can be visualized by the user. Accordingly, when used with a machine programmed with instructions for using the data (e.g., a computer loaded with one or more programs of the sort described herein) the machine is capable of displaying a graphical representation (e.g., a two dimensional graphical representation, a three-dimensional graphical representation) of any of the polypeptides, polypeptide fragments, complexes, or complex fragments described herein.

A display (e.g., a computer display) can show a representation of HIV-1 protease or a complex of HIV-1 protease bound to an inhibitor, e.g., a candidate inhibitor or an inhibitor described herein, or a fragment of either of these complexes. The user can inspect the representation and, using information gained from the representation, generate a model of a complex that includes HIV-1 protease or fragment thereof and a candidate inhibitor, i.e., an inhibitor other than an inhibitor described herein, e.g., an analog of an inhibitor described herein, e.g., an analog of P867883. The model can be generated, for example, by altering a previously existing representation of an HIV-1 protease bound to an inhibitor, e.g., P867883, or a previously existing representation of the active site of an HIV-1 protease bound to an inhibitor, e.g., P867883. Optionally, the user can superimpose a three-dimensional model of a candidate inhibitor on the representation of the active site of an HIV-1 protease bound to an inhibitor, e.g., P867883 or the entire HIV-1 protease bound to an inhibitor, e.g., P867883. In some embodiments, the inhibitor can be a known compound or fragment of a compound. In certain embodiments, the inhibitor can be a previously unknown compound, or a fragment of a previously unknown compound.

It can be desirable for the candidate inhibitor to have a shape that complements the shape of the active site. There can be a preferred distance, or range of distances, between atoms of the candidate inhibitor and atoms of the HIV-1 protease. Distances longer than a preferred distance may be associated with a weak interaction between the candidate inhibitor and the active site of an HIV-1 protease. Distances shorter than a preferred distance may be associated with repulsive forces that can weaken the interaction between the candidate inhibitor and the polypeptide.

A steric clash can occur when distances between atoms are too short. A steric clash occurs when the locations of two atoms are unreasonably close together, for example, when two atoms are separated by a distance less than the sum of their van der Waals radii. If a steric clash exists, the user can adjust the position of the inhibitor relative to the HIV-1 protease (e.g., a rigid body translation or rotation of the inhibitor), until the steric clash is relieved. The user can adjust the conformation or composition of the inhibitor in order to relieve a steric clash. Steric clashes can be removed, for example, by altering the structure of the inhibitor, for example, by changing a “bulky group,” such as an aromatic ring, to a smaller group, such as to a methyl or hydroxyl group, or by changing a rigid group to a flexible group that can accommodate a conformation that does not produce a steric clash.

Electrostatic forces can also influence an interaction between an inhibitor and a ligand-binding domain. For example, electrostatic properties can be associated with repulsive forces that can weaken the interaction between the inhibitor and the HIV-1 protease. Electrostatic repulsion can be relieved by altering the charge of the inhibitor, e.g., by replacing a positively charged group with a neutral group.

Forces that influence binding strength between an inhibitor and HIV-1 protease can be evaluated in the protease/inhibitor model. These can include, for example, hydrogen bonding, electrostatic forces, hydrophobic interactions, van der Waals interactions, dipole-dipole interactions, π-stacking forces, and cation-π interactions. The user can evaluate these forces visually, for example by noting a hydrogen bond donor/acceptor pair arranged with a distance and angle suitable for formation of a hydrogen bond. Based on the evaluation, the user can alter the model to find a more favorable interaction between the HIV-1 protease and the inhibitor.

Altering the model will generally include altering the chemical structure of the inhibitor, for example by substituting, adding, or removing groups. For example, if a hydrogen bond donor on the HIV-1 protease is located near a hydrogen bond donor on the inhibitor, the user can replace the hydrogen bond donor on the inhibitor with a hydrogen bond acceptor.

The relative locations of an inhibitor and the HIV-1 protease, or their conformations, can be adjusted to find an optimized binding geometry for a particular inhibitor to the HIV-1 protease, e.g., within the bounds of the electron density map. An optimized binding geometry is characterized by, for example, favorable hydrogen bond distances and angles, maximal electrostatic attractions, minimal electrostatic repulsions, the sequestration of hydrophobic moieties away from an aqueous environment, and the absence of steric clashes. The optimized geometry can have the lowest calculated energy of a family of possible geometries for an HIV-1 protease/inhibitor complex. An optimized geometry can be determined, for example, through molecular mechanics or molecular dynamics calculations.

A series of representations of complexes of HIV-1 protease bound to different inhibitors, e.g., candidate inhibitors or inhibitors described herein, can be generated. A score can be calculated for each representation. The score can describe, for example, an expected strength of interaction between HIV-1 protease and the inhibitor or inhibitor. The score can reflect one of the factors described above that influence binding strength. The score can be an aggregate score that reflects more than one of the factors. The different inhibitors can be ranked according to their scores.

Steps in the design of a candidate inhibitor can be carried out in an automated fashion by a machine. For example, a representation of HIV-1 protease, or the active site of an HIV-1 protease, can be programmed in the machine, along with representations of candidate inhibitors. The machine can find an optimized binding geometry for each of the candidate inhibitors to the active site, and calculate a score to determine which of the inhibitors in the series is likely to interact most strongly with the active site of the HIV-1 protease.

A software system can be designed and/or implemented to facilitate these steps. Software systems (e.g., computer programs) used to generate representations or perform the fitting analyses include, for example: MCSS, Ludi, QUANTA, Insight II, Cerius2, CHarMM, and Modeler from Accelrys, Inc. (San Diego, Calif.); SYBYL, Unity, F1eXX, and LEAPFROG from TRIPOS, Inc. (St. Louis, Mo.); AUTODOCK (Scripps Research Institute, La Jolla, Calif.); GRID (Oxford University, Oxford, UK); DOCK (University of California, San Francisco, Calif.); and Flo+ and Flo99 (Thistlesoft, Morris Township, N.J.). Other useful programs include ROCS, ZAP, FRED, Vida, and Szybki from Openeye Scientific Software (Santa Fe, N. Mex.); Maestro, Macromodel, and Glide from Schrodinger, LLC (Portland, Oreg.); MOE (Chemical Computing Group, Montreal, Quebec), Allegrow (Boston De Novo, Boston, Mass.), and GOLD (Jones et al., J. Mol. Biol. 245:43-53, 1995). The structural coordinates can also be used to visualize the three-dimensional structure of an ERalpha polypeptide using MOLSCRIPT, RASTER3D, or PYMOL (Kraulis, J. Appl. Crystallogr. 24: 946-950, 1991; Bacon and Anderson, J. Mol. Graph. 6: 219-220, 1998; DeLano, The PyMOL Molecular Graphics System (2002) DeLano Scientific, San Carlos, Calif.).

A candidate inhibitor can, for example, be selected by screening an appropriate database, can be designed de novo by analyzing the steric configurations and charge potentials of unbound HIV-1 protease in conjunction with the appropriate software systems, and/or can be designed using characteristics of known inhibitors, e.g., P867883 or another inhibitor described herein. The method can be used to design or select inhibitors of HIV-1 protease that bind to HIV-1 protease in a manner similar to P867883. A software system can be designed and/or implemented to facilitate database searching, and/or inhibitor selection and design.

Once a candidate inhibitor has been designed or identified, it can be obtained or synthesized and further evaluated for its effect on HIV-1 protease. For example, the inhibitor can be evaluated by contacting it with HIV-1 protease and measuring the effect of the inhibitor on protease activity. A method for evaluating the inhibitor can include an activity assay performed in vitro or in vivo. An activity assay can be a cell-based assay, for example. The candidate inhibitor can also be subjected to cross-resistance profiling, e.g., as described in Petropoulos, Antimicrob Agents Chemother 44:920-8 (2000); and Wu et al., 2006, supra. For example, cross-resistance profiling can be performed using the PhenoSense™ HIV phenotypic drug resistance assay (Monogram Biosciences, Inc., South San Francisco, Calif.) and/or the ANTIVIROGRAM®, a conventional HIV-1 phenotyping assay that uses fully replication-competent recombinant virus to assess the susceptibility to each of the currently available protease and reverse transcriptase inhibitors (Virco BVBA, Mechelen, Belgium).

Depending upon the action of the inhibitor on HIV-1 protease, the inhibitor can be classified as an inhibitor. A crystal containing HIV-1 protease bound to the identified inhibitor can be grown and the structure determined by X-ray crystallography. A second inhibitor can be designed or identified based on the interaction of the first inhibitor with HIV-1 protease.

Various molecular analysis and rational drug design techniques are further disclosed in, for example, U.S. Pat. Nos. 5,834,228, 5,939,528 and 5,856,116, as well as in PCT Application No. PCT/US98/16879, published as WO 99/09148.

### EXAMPLES

The invention is further described in the following examples, which do not limit the scope of the invention described in the claims.

### Example 1

### Crystal Structure of Inhibitor P867883 Bound to HIV-1 Protease

This Example describes the preparation and solution of crystals of P867883 bound to the HIV-1 protease.

Protein Expression and Purification

The wild-type protease was expressed from a synthetic gene optimized for Escherichia coli codon usage with the Gln7Lys mutation to prevent autoproteolysis (Rose, J. R. et al. J Biol Chem 268, 11939-11945, 1993) (shown in SEQ ID NO:1). The protease was expressed and purified as previously described (King, N. M. et al. Protein Sci 11, 418-429, 2002). The protein was refolded by rapid dilution in a 10-fold volume of 0.05 M sodium acetate buffer at pH 5.5, containing 10% glycerol, 5% ethylene glycol and 5 mM dithiothreitol (refolding buffer). To reduce the volume, the protease was concentrated and dialyzed to remove any residual acetic acid. Protease used for crystallization was further purified with a Pharmacia Superdex 75 fast-performance liquid chromatography column equilibrated with refolding buffer.

Crystallization and Data Collection

Crystals were set up with a three-fold molar excess of inhibitor to protease, which ensures ubiquitous binding. The concentration of the protein was 1.6 mg/ml in refolding buffer. The hanging drop method was used for crystallization as previously described (Prabu-Jeyabalan et al. J Virol 77, 1306-1315, 2003). The reservoir solution consisted of 126 mM phosphate buffer at pH 6.2, 63 mM sodium citrate and 26% ammonium sulfate.

Intensity data were collected on an in-house Rigaku X-ray generator equipped with an R-axis IV image plate system. Data were collected at −80° C. Approximately 200 5-minute frames were collected with 1-degree oscillations and no overlap between frames. The frames were integrated and scaled using the programs DENZO (Minor, (Purdue University., West Lafayette, Ind., 1993) and ScalePack (Otwinowski et al., Methods Enzymol 276, 307-326, 1997), respectively. The data collection statistics are listed in Table 1.

TABLE 1
Data Collection Statistics

| | |
|---|---|
| Parameter | P867883 |

Data Collection

| | |
|---|---|
| Space group | P2₁2₁2₁ |
| Z | 4 |
| a (Å) | 51.11 |
| b (Å) | 58.10 |
| c (Å) | 61.60 |
| Resolution (Å) | 2.0 |
| Total number of reflections | 81058 |
| Number of unique reflections | 12879 |
| Rmerge (%) | 7.2 |
| Completeness (%) | 99.2 |
| I/σ_{I} | 13.1 |

Refinement:

| | |
|---|---|
| R_{work} value (%) | 18.7 |
| R_{free} (%) | 23.2 |
| RMSD | |
| Bond length (Å) | 0.007 |
| Bond angles (°) | 1.260 |
| Number of waters | 110 |

Structure Solution and Crystallographic Refinement

The CCP41 interface to the CCP4 suite (Collaborative-Computational-Project, N. The CCP4 suite: programs for protein crystallography. Acta Crystallogr D Biol Crystallogr 50, 760-763, 1994) was used to refine the structure. The structure was solved with the molecular replacement package AMoRe (Navaza, Acta Crystallogr D Biol Crystallogr A50, 157-163, 1994), with 1F7A (Prabu-Jeyabalan, M et al. J Mol Biol 301, 1207-20, 2000) as the starting model. A radius of integration of 25 Å and X-ray data within 8.0 to 3.0 Å were used for the structure solution. The molecular replacement phases were further improved by using ARP/wARP (Morris et al., Acta Crystallogr D Biol Crystallogr D58, 968-975, 2002) to build solvent molecules into the unaccounted regions of electron density. Difference Fourier maps were computed and inspected with the interactive graphic program 0 (Jones et al., Methods in Molecular Design (eds. Bugg and Ealick) 189-195 (Springer-Verlag Press, Berlin, 1990), and major structural changes were incorporated in the model, such as inclusion of inhibitor and solvent molecules. Conjugate gradient refinement using Refmac5 (Murshudov et al., Acta Crystallogr D Biol Crystallogr D53, 240-255, 1997) was performed by incorporating the Schomaker and Trueblood tensor formulation of TLS (translation, libration, screw-rotation) parameters (Kuriyan and Weis, Proc Natl Acad Sci USA 88, 2773-2777, 1991, Schomaker and Trueblood, Acta Crystallogr B24, 63-76, 1968, Tickle and Moss, in IUCr99 Computing School IUCr, London, 1999). The working R (R_{factor} 1 and its cross validation (R_{free}) were monitored throughout the refinement.

Results

The crystal structure of the wild-type protease-P867883 complex, which crystallized in P2₁2₁2₁ space group with one protease dimer per asymmetric unit, was solved and refined to 2.0 Å. The inhibitor was modeled in one orientation, with continuous electron density for the entire molecule except for the isopropyl group at P1′. The final R_{factor} value is 18.7% (R_{free}=23.2%). The crystallographic statistics are listed in Table 1. The atomic coordinates are listed in Table 2.

P867883 binds to the active site through interactions between two oxygen atoms of the inhibitor's sulfonyl group and the nitrogen atoms of protease's Ile50 and Ile50′. In other protease-inhibitor and protease-substrate complexes these interactions are made by a conserved water molecule to the nitrogen atoms of Ile50 and Ile50′, indicating a novel mode of binding for P867883.

P867883 protease hydrogen bonds. P867883 displays a novel hydrogen bonding pattern compared to other protease inhibitors. FIG. 2B shows hydrogen bonding between atoms in the protease active site and atoms in the inhibitor, including those mediated by water. The catalytic aspartic acids of the enzyme, Asp25 and Asp25′, are within hydrogen bonding distance of the hydroxyl group of P867883. The inhibitor makes 12 hydrogen bonds with the protease. Except for one interaction, all hydrogen bonds are between the inhibitor and either protease main-chain atoms or side-chain atoms of conserved residues (Asp25 and Asp29). The two nitrogen atoms of Ile50 and Ile50′ at the tips of the flaps form hydrogen bonds with the two oxygen atoms of the inhibitor's sulfonyl group. This hydrogen bonding is a novel structural feature compared to the structures of most protease-inhibitor complexes in which a water molecule tetrahedrally coordinates the nitrogen atoms of Ile50 and Ile50′ with the inhibitor atoms.

The amide and carbamate groups of P867883 form hydrogen bonds with residue Gly48 in the flap and with residue Asp29 in the floor of the active site. At the other end of the inhibitor, the amine group forms a hydrogen bond with the carboxyl group of Asp30′. This amine group also forms water-mediated hydrogen bonds with the residues Gly48′ in the flap and Asp29′ at the bottom of the active site. The important feature of the P867883-protease hydrogen bonds is their involvement with residues in both the flap (Gly48 and Ile50) and the active site (Asp25, Asp29 and Asp30). This feature is distinct from the inhibitor-protease hydrogen bonds formed by peptidomimetic inhibitors (IDV, NFV, DRV, APV, LPV), which do not form hydrogen bonds with the flap residues (Prabu-Jeyabalan et al., Antimicrob Agents Chemother 50, 1518-1521, 2006). The substrates, in contrast, form hydrogen bonds with the flap residue, Gly48, and in certain cases, even with Met46 (Prabu-Jeyabalan et al., Structure 10, 369-381, 2002).

van der Waals contacts. P867883 packs in an extended conformation in the active site by forming 134 van der Waals (vdW) contacts to the protease, with an interatomic distance of <4.2 Å (FIG. 2C). The isopropyl group of the P1′ site is surrounded by residues Leu23, Asp25, Ile84, Gly27′ and Ile50′ of the protease. The benzyl amine group of P2′ forms vdW contacts with Ile50, Gly48′, Gly49′ of the flap as well as with Asp30′ and Val32′. The aliphatic hydrophobic amino alkyl central part of the inhibitor backbone is within vdW distance of the aliphatic hydrophobic residues Ala28, Ile47, Gly48, Ile50′, Ile84. An aromatic diphenyl methyl group occupies P3 position. One of the phenyl rings of this group points in the direction of the P1 site. This phenyl ring is within vdW distance of many protease residues (Leu23′, Asp25′, Gly27, Gly48, Gly49, Ile50, Pro81′, Ile84′), whereas the other phenyl ring forms vdW contacts with only three residues (Arg8′, Pro81′, Val82′).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

TABLE 2

| | | | |
|---|---|---|---|
| HEADER | --- | XX-XXX-XX | MADH |
| COMPND | --- | | |

| | | | |
|---|---|---|---|
| REMARK | 3 | | |
| REMARK | 3 | REFINEMENT. | |
| REMARK | 3 | PROGRAM: | REFMAC 5.2.0005 |
| REMARK | 3 | AUTHORS: | MURSHUDOV, VAGIN, DODSON |
| REMARK | 3 | | |

| REMARK | 3 | REFINEMENT TARGET: MAXIMUM LIKELIHOOD | | |
|---|---|---|---|---|
| REMARK | 3 | | | |
| REMARK | 3 | DATA USED IN REFINEMENT. | | |
| REMARK | 3 | RESOLUTION RANGE HIGH | (ANGSTROMS): | 2.00 |
| REMARK | 3 | RESOLUTION RANGE LOW | (ANGSTROMS): | 42.26 |
| REMARK | 3 | DATA CUTOFF | (SIGMA(F)): | NONE |
| REMARK | 3 | COMPLETENESS FOR RANGE | (%): | 98.82 |
| REMARK | 3 | NUMBER OF REFLECTIONS: | | 12220 |
| REMARK | 3 | | | |

| REMARK | 3 | FIT TO DATA USED IN REFINEMENT. | | |
|---|---|---|---|---|
| REMARK | 3 | CROSS-VALIDATION METHOD: | | THROUGHOUT |
| REMARK | 3 | FREE R VALUE TEST SET SELECTION: | | RANDOM |
| REMARK | 3 | R VALUE | (WORKING + TEST SET): | 0.18898 |
| REMARK | 3 | R VALUE | (WORKING SET): | 0.18676 |
| REMARK | 3 | FREE R VALUE: | | 0.23230 |

| | | | | |
|---|---|---|---|---|
| REMARK | 3 | FREE R VALUE TEST SET SIZE | (%): | 4.8 |

| | | | |
|---|---|---|---|
| REMARK | 3 | FREE R VALUE TEST SET COUNT: | 619 |
| REMARK | 3 | | |
| REMARK | 3 | FIT IN THE HIGHEST RESOLUTION BIN. | |

| REMARK | 3 | TOTAL NUMBER OF BINS USED: | | 20 |
|---|---|---|---|---|
| REMARK | 3 | BIN RESOLUTION RANGE HIGH: | | 1.995 |
| REMARK | 3 | BIN RESOLUTION RANGE LOW: | | 2.047 |
| REMARK | 3 | REFLECTION IN BIN | (WORKING SET): | 844 |
| REMARK | 3 | BIN COMPLETENESS | (WORKING + TEST) (%): | 94.32 |
| REMARK | 3 | BIN R VALUE | (WORKING SET): | 0.219 |

| | | | |
|---|---|---|---|
| REMARK | 3 | BIN FREE R VALUE SET COUNT: | 36 |
| REMARK | 3 | BIN FREE R VALUE: | 0.312 |
| REMARK | 3 | | |

| REMARK | 3 | NUMBER OF NON-HYDROGEN ATOMS USED IN REFINEMENT. | |
|---|---|---|---|
| REMARK | 3 | ALL ATOMS: | 1679 |
| REMARK | 3 | | |
| REMARK | 3 | B VALUES. | |

| | | | | |
|---|---|---|---|---|
| REMARK | 3 | FROM WILSON PLOT | (A**2): | NULL |
| REMARK | 3 | MEAN B VALUE | (OVERALL, A**2): | 24.819 |

| | | |
|---|---|---|
| REMARK | 3 | OVERALL ANISOTROPIC B VALUE. |

| | | | |
|---|---|---|---|
| REMARK | 3 | B11 (A**2): | −1.10 |
| REMARK | 3 | B22 (A**2): | −0.32 |
| REMARK | 3 | B33 (A**2): | 1.42 |
| REMARK | 3 | B12 (A**2): | 0.00 |
| REMARK | 3 | B13 (A**2): | 0.00 |
| REMARK | 3 | B23 (A**2): | 0.00 |
| REMARK | 3 | | |

| | | | | |
|---|---|---|---|---|
| REMARK | 3 | ESTIMATED OVERALL COORDINATE ERROR. | | |
| REMARK | 3 | ESU BASED ON R VALUE | (A): | 0.210 |
| REMARK | 3 | ESU BASED ON FREE R VALUE | (A): | 0.175 |
| REMARK | 3 | ESU BASED ON MAXIMUM LIKELIHOOD | (A): | 0.127 |
| REMARK | 3 | ESU FOR B VALUES BASED ON MAXIMUM LIKELIHOOD | (A**2): | 8.829 |
| REMARK | 3 | | | |
| REMARK | 3 | CORRELATION COEFFICIENTS. | | |

| REMARK | 3 | CORRELATION COEFFICIENT FO-FC: | 0.954 | | | |
|---|---|---|---|---|---|---|
| REMARK | 3 | CORRELATION COEFFICIENT FO-FC FREE: | 0.935 | | | |
| REMARK | 3 | | | | | |
| REMARK | 3 | RMS DEVIATIONS FROM IDEAL VALUES | | COUNT | RMS | WEIGHT |
| REMARK | 3 | BOND LENGTHS REFINED ATOMS | (A): | 1593; | 0.007; | 0.022 |
| REMARK | 3 | BOND LENGTHS OTHERS | (A): | 1534; | 0.001; | 0.020 |
| REMARK | 3 | BOND ANGLES REFINED ATOMS | (DEGREES): | 2169; | 1.260; | 2.025 |
| REMARK | 3 | BOND ANGLES OTHERS | (DEGREES): | 3552; | 0.647; | 3.000 |
| REMARK | 3 | TORSION ANGLES, PERIOD 1 | (DEGREES): | 200; | 6.588; | 5.000 |
| REMARK | 3 | TORSION ANGLES, PERIOD 2 | (DEGREES): | 52; | 37.808; | 25.000 |
| REMARK | 3 | TORSION ANGLES, PERIOD 3 | (DEGREES): | 260; | 12.364; | 15.000 |
| REMARK | 3 | TORSION ANGLES, PERIOD 4 | (DEGREES): | 7; | 14.960; | 15.000 |
| REMARK | 3 | CHIRAL-CENTER RESTRAINTS | (A**3): | 256; | 0.080; | 0.200 |
| REMARK | 3 | GENERAL PLANES REFINED ATOMS | (A): | 1727; | 0.006; | 0.020 |
| REMARK | 3 | GENERAL PLANES OTHERS | (A): | 282; | 0.001; | 0.020 |
| REMARK | 3 | NON-BONDED CONTACTS REFINED ATOMS | (A): | 234; | 0.174; | 0.200 |
| REMARK | 3 | NON-BONDED CONTACTS OTHERS | (A): | 1559; | 0.188; | 0.200 |
| REMARK | 3 | NON-BONDED TORSION REFINED ATOMS | (A): | 741; | 0.175; | 0.200 |
| REMARK | 3 | NON-BONDED TORSION OTHERS | (A): | 1025; | 0.085; | 0.200 |
| REMARK | 3 | H-BOND (X...Y) REFINED ATOMS | (A): | 80; | 0.177; | 0.200 |
| REMARK | 3 | SYMMETRY VDW REFINED ATOMS | (A): | 11; | 0.289; | 0.200 |
| REMARK | 3 | SYMMETRY VDW OTHERS | (A): | 61; | 0.205; | 0.200 |
| REMARK | 3 | SYMMETRY H-BOND REFINED ATOMS | (A): | 8; | 0.282; | 0.200 |
| REMARK | 3 | | | | | |
| REMARK | 3 | ISOTROPIC THERMAL FACTOR RESTRAINTS. | | COUNT | RMS | WEIGHT |
| REMARK | 3 | MAIN-CHAIN BOND REFINED ATOMS | (A**2): | 1059; | 0.743; | 1.500 |
| REMARK | 3 | MAIN-CHAIN BOND OTHER ATOMS | (A**2): | 416; | 0.196; | 1.500 |
| REMARK | 3 | MAIN-CHAIN ANGLE REFINED ATOMS | (A**2): | 1614; | 0.960; | 2.000 |
| REMARK | 3 | SIDE-CHAIN BOND REFINED ATOMS | (A**2): | 647; | 1.519; | 3.000 |
| REMARK | 3 | SIDE-CHAIN ANGLE REFINED ATOMS | (A**2): | 555; | 2.208; | 4.500 |
| REMARK | 3 | | | | | |

| REMARK | 3 | NCS RESTRAINTS STATISTICS | | |
|---|---|---|---|---|
| REMARK | 3 | NUMBER OF NCS GROUPS: NULL | | |
| REMARK | 3 | | | |
| REMARK | 3 | | | |
| REMARK | 3 | TLS DETAILS | | |
| REMARK | 3 | NUMBER OF TLS GROUPS: 21 | | |
| REMARK | 3 | ATOM RECORD CONTAINS RESIDUAL B FACTORS ONLY | | |
| REMARK | 3 | | | |
| REMARK | 3 | TLS GROUP: 1 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 1 | A 7 |
| REMARK | 3 | ORIGIN FOR THE GROUP (A): 20.2533 14.1639 22.3260 | | |
| REMARK | 3 | T TENSOR | | |
| REMARK | 3 | T11: 0.0088 T22: −0.0757 | | |
| REMARK | 3 | T33: 0.0032 T12: −0.0134 | | |
| REMARK | 3 | T13: 0.0506 T23: −0.0236 | | |
| REMARK | 3 | L TENSOR | | |
| REMARK | 3 | L11: 7.2367 L22: 5.8335 | | |
| REMARK | 3 | L33: 8.2982 L12: −4.4678 | | |
| REMARK | 3 | L13: −2.5184 L23: 1.4125 | | |
| REMARK | 3 | S TENSOR | | |
| REMARK | 3 | S11: −0.0791 S12: 0.1776 S13: −0.8795 | | |
| REMARK | 3 | S21: 0.9283 S22: −0.0128 S23: 0.6753 | | |
| REMARK | 3 | S31: 0.6836 S32: −0.6229 S33: 0.0920 | | |
| REMARK | 3 | | | |
| REMARK | 3 | TLS GROUP: 2 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 8 | A 12 |
| REMARK | 3 | ORIGIN FOR THE GROUP (A): 13.4279 18.2148 16.0872 | | |
| REMARK | 3 | T TENSOR | | |
| REMARK | 3 | T11: −0.0742 T22: −0.1087 | | |
| REMARK | 3 | T33: 0.0117 T12: 0.0132 | | |
| REMARK | 3 | T13: −0.0238 T23: −0.0240 | | |
| REMARK | 3 | L TENSOR | | |
| REMARK | 3 | L11: 53.3065 L22: 9.9464 | | |
| REMARK | 3 | L33: 1.7758 L12: −4.6868 | | |
| REMARK | 3 | L13: −4.6541 L23: −0.5984 | | |
| REMARK | 3 | S TENSOR | | |
| REMARK | 3 | S11: −0.0951 S12: 1.6104 S13: −0.6781 | | |
| REMARK | 3 | S21: −0.1481 S22: 0.0634 S23: 0.8956 | | |
| REMARK | 3 | S31: 0.0656 S32: −0.5675 S33: 0.0316 | | |
| REMARK | 3 | | | |
| REMARK | 3 | TLS GROUP: 3 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 13 | A 26 |
| REMARK | 3 | ORIGIN FOR THE GROUP (A): 6.5613 22.3590 16.8042 | | |
| REMARK | 3 | T TENSOR | | |
| REMARK | 3 | T11: −0.0690 T22: −0.0738 | | |
| REMARK | 3 | T33: 0.0639 T12: −0.0085 | | |
| REMARK | 3 | T13: 0.0149 T23: −0.0296 | | |
| REMARK | 3 | L TENSOR | | |
| REMARK | 3 | L11: 28.5732 L22: 1.6520 | | |
| REMARK | 3 | L33: 5.4724 L12: 3.4678 | | |
| REMARK | 3 | L13: 7.9967 L23: 2.1138 | | |
| REMARK | 3 | S TENSOR | | |
| REMARK | 3 | S11: 0.1614 S12: 0.6621 S13: −0.9402 | | |
| REMARK | 3 | S21: −0.0359 S22: 0.0032 S23: 0.4095 | | |
| REMARK | 3 | S31: 0.1956 S32: −0.4190 S33: −0.1646 | | |
| REMARK | 3 | | | |
| REMARK | 3 | TLS GROUP: 4 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 27 | A 33 |
| REMARK | 3 | ORIGIN FOR THE GROUP (A): 12.5642 32.1612 18.0832 | | |
| REMARK | 3 | T TENSOR | | |
| REMARK | 3 | T11: −0.0854 T22: −0.1178 | | |
| REMARK | 3 | T33: 0.0032 T12: 0.0257 | | |
| REMARK | 3 | T13: −0.0208 T23: −0.0325 | | |
| REMARK | 3 | L TENSOR | | |
| REMARK | 3 | L11: 1.6373 L22: 0.1812 | | |
| REMARK | 3 | L33: 11.7405 L12: −0.5447 | | |
| REMARK | 3 | L13: 4.3844 L23: −1.4585 | | |
| REMARK | 3 | S TENSOR | | |
| REMARK | 3 | S11: −0.1072 S12: −0.3691 S13: 0.2231 | | |
| REMARK | 3 | S21: −0.0220 S22: 0.1583 S23: 0.4399 | | |
| REMARK | 3 | S31: −0.6264 S32: −0.4651 S33: −0.0511 | | |
| REMARK | 3 | | | |
| REMARK | 3 | TLS GROUP: 5 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 34 | A 37 |
| REMARK | 3 | ORIGIN FOR THE GROUP (A): 1.9732 30.0448 9.2304 | | |
| REMARK | 3 | T TENSOR | | |
| REMARK | 3 | T11: 0.0853 T22: 0.1024 | | |
| REMARK | 3 | T33: 0.0375 T12: −0.1363 | | |
| REMARK | 3 | T13: −0.1390 T23: 0.0459 | | |
| REMARK | 3 | L TENSOR | | |
| REMARK | 3 | L11: 42.3118 L22: 55.6425 | | |
| REMARK | 3 | L33: 19.9083 L12: −39.7854 | | |
| REMARK | 3 | L13: −18.3235 L23: 3.4888 | | |
| REMARK | 3 | S TENSOR | | |
| REMARK | 3 | S11: 0.2342 S12: 1.0275 S13: −0.3756 | | |
| REMARK | 3 | S21: −0.3264 S22: −0.1747 S23: 1.0820 | | |
| REMARK | 3 | S31: 1.2727 S32: −1.3739 S33: −0.0595 | | |
| REMARK | 3 | | | |
| REMARK | 3 | TLS GROUP: 6 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 38 | A 45 |
| REMARK | 3 | ORIGIN FOR THE GROUP (A): 1.4368 39.9831 14.5142 | | |
| REMARK | 3 | T TENSOR | | |
| REMARK | 3 | T11: −0.0964 T22: 0.0171 | | |
| REMARK | 3 | T33: 0.2971 T12: 0.0558 | | |
| REMARK | 3 | T13: 0.0945 T23: 0.0757 | | |
| REMARK | 3 | L TENSOR | | |
| REMARK | 3 | L11: 26.9281 L22: 22.0339 | | |
| REMARK | 3 | L33: 2.9887 L12: 17.4593 | | |
| REMARK | 3 | L13: −6.3171 L23: −8.1137 | | |
| REMARK | 3 | S TENSOR | | |
| REMARK | 3 | S11: 0.3495 S12: −0.4777 S13: 2.4648 | | |
| REMARK | 3 | S21: 0.7240 S22: 0.7035 S23: 2.0061 | | |
| REMARK | 3 | S31: −0.1871 S32: −0.4257 S33: −1.0530 | | |
| REMARK | 3 | | | |
| REMARK | 3 | TLS GROUP: 7 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 46 | A 50 |
| REMARK | 3 | ORIGIN FOR THE GROUP (A): 17.2779 37.7842 11.9730 | | |
| REMARK | 3 | T TENSOR | | |
| REMARK | 3 | T11: −0.0395 T22: −0.0505 | | |
| REMARK | 3 | T33: −0.1297 T12: 0.0397 | | |
| REMARK | 3 | T13: 0.0382 T23: 0.0033 | | |
| REMARK | 3 | L TENSOR | | |
| REMARK | 3 | L11: 83.4841 L22: 47.2632 | | |
| REMARK | 3 | L33: 2.9140 L12: −52.9502 | | |
| REMARK | 3 | L13: 3.9398 L23: 3.6100 | | |
| REMARK | 3 | S TENSOR | | |
| REMARK | 3 | S11: 0.4329 S12: −0.2094 S13: −0.4895 | | |
| REMARK | 3 | S21: −0.9040 S22: 0.0805 S23: 0.5825 | | |
| REMARK | 3 | S31: 0.2856 S32: 0.3092 S33: −0.5134 | | |
| REMARK | 3 | | | |
| REMARK | 3 | TLS GROUP: 8 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 51 | A 74 |
| REMARK | 3 | ORIGIN FOR THE GROUP (A): 5.5620 32.1079 18.8720 | | |
| REMARK | 3 | T TENSOR | | |
| REMARK | 3 | T11: −0.0609 T22: −0.0407 | | |
| REMARK | 3 | T33: 0.0590 T12: 0.0157 | | |
| REMARK | 3 | T13: −0.0118 T23: 0.0962 | | |
| REMARK | 3 | L TENSOR | | |
| REMARK | 3 | L11: 6.1367 L22: 4.5073 | | |
| REMARK | 3 | L33: 6.4415 L12: 3.5409 | | |
| REMARK | 3 | L13: −4.1492 L23: −5.3874 | | |
| REMARK | 3 | S TENSOR | | |
| REMARK | 3 | S11: 0.2060 S12: −0.2608 S13: 0.4530 | | |
| REMARK | 3 | S21: −0.1078 S22: 0.1981 S23: 0.6654 | | |
| REMARK | 3 | S31: 0.1840 S32: −0.0602 S33: −0.4040 | | |
| REMARK | 3 | | | |
| REMARK | 3 | TLS GROUP: 9 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 75 | A 88 |
| REMARK | 3 | ORIGIN FOR THE GROUP (A): 10.5467 30.2638 15.1896 | | |
| REMARK | 3 | T TENSOR | | |
| REMARK | 3 | T11: −0.0934 T22: −0.0781 | | |
| REMARK | 3 | T33: −0.0040 T12: 0.0263 | | |
| REMARK | 3 | T13: −0.0032 T23: 0.0216 | | |
| REMARK | 3 | L TENSOR | | |
| REMARK | 3 | L11: 5.1046 L22: 5.7971 | | |
| REMARK | 3 | L33: 5.0675 L12: −3.2991 | | |
| REMARK | 3 | L13: 3.9947 L23: −5.2492 | | |
| REMARK | 3 | S TENSOR | | |
| REMARK | 3 | S11: 0.2465 S12: 0.2892 S13: 0.4017 | | |
| REMARK | 3 | S21: −0.5560 S22: −0.2905 S23: 0.3795 | | |
| REMARK | 3 | S31: 0.4765 S32: 0.1718 S33: 0.0439 | | |
| REMARK | 3 | | | |
| REMARK | 3 | TLS GROUP: 10 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | A 89 | A 99 |
| REMARK | 3 | ORIGIN FOR THE GROUP (A): 15.9880 24.3215 28.7317 | | |
| REMARK | 3 | T TENSOR | | |
| REMARK | 3 | T11: −0.0184 T22: 0.0238 | | |
| REMARK | 3 | T33: −0.0866 T12: 0.0476 | | |
| REMARK | 3 | T13: 0.1024 T23: 0.0264 | | |
| REMARK | 3 | L TENSOR | | |
| REMARK | 3 | L11: 11.8857 L22: 8.8690 | | |
| REMARK | 3 | L33: 2.6154 L12: −8.8530 | | |
| REMARK | 3 | L13: 4.0519 L23: −1.8852 | | |
| REMARK | 3 | S TENSOR | | |
| REMARK | 3 | S11: −0.3704 S12: −0.3117 S13: −0.3156 | | |
| REMARK | 3 | S21: 0.5457 S22: 0.1360 S23: 0.3309 | | |
| REMARK | 3 | S31: 0.1590 S32: −0.1623 S33: 0.2345 | | |
| REMARK | 3 | | | |
| REMARK | 3 | TLS GROUP: 11 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 1 | B 7 |
| REMARK | 3 | ORIGIN FOR THE GROUP (A): 19.8953 27.8418 31.9033 | | |
| REMARK | 3 | T TENSOR | | |
| REMARK | 3 | T11: 0.1136 T22: 0.0499 | | |
| REMARK | 3 | T33: −0.1241 T12: 0.0609 | | |
| REMARK | 3 | T13: −0.0149 T23: −0.0795 | | |
| REMARK | 3 | L TENSOR | | |
| REMARK | 3 | L11: 9.5293 L22: 8.8408 | | |
| REMARK | 3 | L33: 2.6999 L12: −1.3452 | | |
| REMARK | 3 | L13: −2.9545 L23: −0.9705 | | |
| REMARK | 3 | S TENSOR | | |
| REMARK | 3 | S11: −0.2714 S12: −1.0681 S13: 0.1374 | | |
| REMARK | 3 | S21: 1.4438 S22: 0.3372 S23: −0.2156 | | |
| REMARK | 3 | S31: 0.3511 S32: 0.0013 S33: −0.0659 | | |
| REMARK | 3 | | | |
| REMARK | 3 | TLS GROUP: 12 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 8 | B 13 |
| REMARK | 3 | ORIGIN FOR THE GROUP (A): 28.1340 32.1189 25.7782 | | |
| REMARK | 3 | T TENSOR | | |
| REMARK | 3 | T11: −0.0560 T22: −0.0610 | | |
| REMARK | 3 | T33: −0.1372 T12: 0.0460 | | |
| REMARK | 3 | T13: 0.0027 T23: −0.0489 | | |
| REMARK | 3 | L TENSOR | | |
| REMARK | 3 | L11: 34.5010 L22: 2.5197 | | |
| REMARK | 3 | L33: 0.0930 L12: 1.6655 | | |
| REMARK | 3 | L13: 0.1730 L23: 0.4825 | | |
| REMARK | 3 | S TENSOR | | |
| REMARK | 3 | S11: 0.4725 S12: −0.3054 S13: 0.5007 | | |
| REMARK | 3 | S21: 0.2123 S22: −0.3302 S23: −0.0755 | | |
| REMARK | 3 | S31: −0.4414 S32: −0.0315 S33: −0.1422 | | |
| REMARK | 3 | | | |
| REMARK | 3 | TLS GROUP: 13 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 14 | B 20 |
| REMARK | 3 | ORIGIN FOR THE GROUP (A): 40.9424 31.6242 22.7181 | | |
| REMARK | 3 | T TENSOR | | |
| REMARK | 3 | T11: −0.0873 T22: −0.1326 | | |
| REMARK | 3 | T33: −0.0436 T12: −0.0143 | | |
| REMARK | 3 | T13: −0.0424 T23: 0.0320 | | |
| REMARK | 3 | L TENSOR | | |
| REMARK | 3 | L11: 37.2815 L22: 17.4530 | | |
| REMARK | 3 | L33: 22.7316 L12: −0.6723 | | |
| REMARK | 3 | L13: −13.2444 L23: 6.9131 | | |
| REMARK | 3 | S TENSOR | | |
| REMARK | 3 | S11: −0.2339 S12: 0.1492 S13: 0.9680 | | |
| REMARK | 3 | S21: −0.2042 S22: 0.2664 S23: −1.3109 | | |
| REMARK | 3 | S31: 0.2100 S32: 0.7640 S33: −0.0325 | | |
| REMARK | 3 | | | |
| REMARK | 3 | TLS GROUP: 14 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 21 | B 29 |
| REMARK | 3 | ORIGIN FOR THE GROUP (A): 25.3141 27.5563 19.8226 | | |
| REMARK | 3 | T TENSOR | | |
| REMARK | 3 | T11: −0.1413 T22: −0.1095 | | |
| REMARK | 3 | T33: −0.1323 T12: 0.0237 | | |
| REMARK | 3 | T13: 0.0110 T23: 0.0147 | | |
| REMARK | 3 | L TENSOR | | |
| REMARK | 3 | L11: 8.5013 L22: 2.9393 | | |
| REMARK | 3 | L33: 2.1853 L12: 3.0081 | | |
| REMARK | 3 | L13: 0.3365 L23: −1.8989 | | |
| REMARK | 3 | S TENSOR | | |
| REMARK | 3 | S11: −0.2280 S12: 0.1831 S13: 0.3941 | | |
| REMARK | 3 | S21: 0.0366 S22: −0.0337 S23: 0.3035 | | |
| REMARK | 3 | S31: −0.0327 S32: 0.0482 S33: 0.2618 | | |
| REMARK | 3 | | | |
| REMARK | 3 | TLS GROUP: 15 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 30 | B 35 |
| REMARK | 3 | ORIGIN FOR THE GROUP (A): 32.1899 31.0655 13.3676 | | |
| REMARK | 3 | T TENSOR | | |
| REMARK | 3 | T11: −0.1111 T22: −0.0789 | | |
| REMARK | 3 | T33: −0.1936 T12: 0.0225 | | |
| REMARK | 3 | T13: 0.0281 T23: 0.0263 | | |
| REMARK | 3 | L TENSOR | | |
| REMARK | 3 | L11: 25.7761 L22: 41.7454 | | |
| REMARK | 3 | L33: 2.2628 L12: 22.7500 | | |
| REMARK | 3 | L13: 2.5528 L23: −3.6390 | | |
| REMARK | 3 | S TENSOR | | |
| REMARK | 3 | S11: −0.6481 S12: 0.3468 S13: 0.3601 | | |
| REMARK | 3 | S21: −0.9276 S22: 0.5748 S23: −0.2147 | | |
| REMARK | 3 | S31: −0.4300 S32: −0.2581 S33: 0.0732 | | |
| REMARK | 3 | | | |
| REMARK | 3 | TLS GROUP: 16 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 36 | B 48 |
| REMARK | 3 | ORIGIN FOR THE GROUP (A): 36.9994 28.5830 5.8803 | | |
| REMARK | 3 | T TENSOR | | |
| REMARK | 3 | T11: −0.0205 T22: −0.0930 | | |
| REMARK | 3 | T33: 0.0350 T12: 0.0157 | | |
| REMARK | 3 | T13: 0.0591 T23: 0.0453 | | |
| REMARK | 3 | L TENSOR | | |
| REMARK | 3 | L11: 10.5790 L22: 6.0951 | | |
| REMARK | 3 | L33: 10.1633 L12: 0.5094 | | |
| REMARK | 3 | L13: 5.5884 L23: 2.7850 | | |
| REMARK | 3 | S TENSOR | | |
| REMARK | 3 | S11: 0.0450 S12: 0.5476 S13: −0.5923 | | |
| REMARK | 3 | S21: −0.1093 S22: −0.0038 S23: −0.5972 | | |
| REMARK | 3 | S31: 0.1146 S32: 0.7845 S33: −0.0412 | | |
| REMARK | 3 | | | |
| REMARK | 3 | TLS GROUP: 17 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 49 | B 55 |
| REMARK | 3 | ORIGIN FOR THE GROUP (A): 23.0944 32.4429 4.5301 | | |
| REMARK | 3 | T TENSOR | | |
| REMARK | 3 | T11: 0.1998 T22: 0.3634 | | |
| REMARK | 3 | T33: 0.2279 T12: 0.0374 | | |
| REMARK | 3 | T13: 0.1457 T23: 0.0773 | | |
| REMARK | 3 | L TENSOR | | |
| REMARK | 3 | L11: 75.7687 L22: 4.7251 | | |
| REMARK | 3 | L33: 9.3831 L12: −18.9212 | | |
| REMARK | 3 | L13: −26.6636 L23: 6.6585 | | |
| REMARK | 3 | S TENSOR | | |
| REMARK | 3 | S11: −1.2704 S12: 1.4611 S13: −0.4476 | | |
| REMARK | 3 | S21: −0.1493 S22: 0.2872 S23: 0.9662 | | |
| REMARK | 3 | S31: 0.9230 S32: −1.8269 S33: 0.9832 | | |
| REMARK | 3 | | | |
| REMARK | 3 | TLS GROUP: 18 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 56 | B 66 |
| REMARK | 3 | ORIGIN FOR THE GROUP (A): 38.0651 24.9094 13.8811 | | |
| REMARK | 3 | T TENSOR | | |
| REMARK | 3 | T11: −0.1241 T22: −0.1114 | | |
| REMARK | 3 | T33: −0.0799 T12: −0.0530 | | |
| REMARK | 3 | T13: 0.0139 T23: −0.0300 | | |
| REMARK | 3 | L TENSOR | | |
| REMARK | 3 | L11: 4.0187 L22: 3.1182 | | |
| REMARK | 3 | L33: 17.2205 L12: −2.8640 | | |
| REMARK | 3 | L13: 0.6512 L23: −4.7576 | | |
| REMARK | 3 | S TENSOR | | |
| REMARK | 3 | S11: 0.1899 S12: 0.1379 S13: 0.0871 | | |
| REMARK | 3 | S21: −0.0456 S22: −0.3404 S23: −0.2323 | | |
| REMARK | 3 | S31: 0.0821 S32: 0.4026 S33: 0.1505 | | |
| REMARK | 3 | | | |
| REMARK | 3 | TLS GROUP: 19 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 67 | B 73 |
| REMARK | 3 | ORIGIN FOR THE GROUP (A): 36.7803 20.7098 24.6536 | | |
| REMARK | 3 | T TENSOR | | |
| REMARK | 3 | T11: −0.0830 T22: −0.1040 | | |
| REMARK | 3 | T33: −0.0822 T12: −0.0232 | | |
| REMARK | 3 | T13: −0.0086 T23: 0.0396 | | |
| REMARK | 3 | L TENSOR | | |
| REMARK | 3 | L11: 10.4856 L22: 5.0424 | | |
| REMARK | 3 | L33: 46.4834 L12: −2.9285 | | |
| REMARK | 3 | L13: −13.4444 L23: 11.9689 | | |
| REMARK | 3 | S TENSOR | | |
| REMARK | 3 | S11: −0.0882 S12: −0.8736 S13: 0.2583 | | |
| REMARK | 3 | S21: 0.4818 S22: −0.0638 S23: −0.4751 | | |
| REMARK | 3 | S31: 0.0251 S32: 0.6484 S33: 0.1520 | | |
| REMARK | 3 | | | |
| REMARK | 3 | TLS GROUP: 20 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | B 74 | B 99 |
| REMARK | 3 | ORIGIN FOR THE GROUP (A): 27.4628 24.2151 18.8215 | | |
| REMARK | 3 | T TENSOR | | |
| REMARK | 3 | T11: −0.1148 T22: −0.0916 | | |
| REMARK | 3 | T33: −0.1282 T12: 0.0020 | | |
| REMARK | 3 | T13: −0.0046 T23: 0.0285 | | |
| REMARK | 3 | L TENSOR | | |
| REMARK | 3 | L11: 1.7974 L22: 3.1127 | | |
| REMARK | 3 | L33: 1.5086 L12: −0.3008 | | |
| REMARK | 3 | L13: −0.4493 L23: 0.2052 | | |
| REMARK | 3 | S TENSOR | | |
| REMARK | 3 | S11: −0.1373 S12: 0.0494 S13: −0.0451 | | |
| REMARK | 3 | S21: 0.1427 S22: 0.0985 S23: 0.1983 | | |
| REMARK | 3 | S31: −0.0950 S32: −0.1207 S33: 0.0388 | | |
| REMARK | 3 | | | |
| REMARK | 3 | TLS GROUP: 21 | | |
| REMARK | 3 | NUMBER OF COMPONENTS GROUP: 1 | | |
| REMARK | 3 | COMPONENTS | C SSSEQI TO | C SSSEQI |
| REMARK | 3 | RESIDUE RANGE: | C 200 | C 200 |

| REMARK | 3 | ORIGIN FOR THE GROUP (A): 20.1342 32.5388 14.6692 | |
|---|---|---|---|
| REMARK | 3 | T TENSOR | |
| REMARK | 3 | T11: −0.0622 T22: −0.1177 | |
| REMARK | 3 | T33: −0.0311 T12: 0.0033 | |
| REMARK | 3 | T13: 0.0213 T23: 0.0456 | |
| REMARK | 3 | L TENSOR | |
| REMARK | 3 | L11: 30.0146 L22: 29.7565 | |
| REMARK | 3 | L33: 48.6484 L12: −28.9960 | |
| REMARK | 3 | L13: −27.1396 L23: 30.0094 | |
| REMARK | 3 | S TENSOR | |
| REMARK | 3 | S11: 0.3764 S12: 0.6733 S13: 0.6460 | |
| REMARK | 3 | S21: −0.9054 S22: 0.2589 S23: −0.8331 | |
| REMARK | 3 | S31: −0.4749 S32: −0.2191 S33: −0.6353 | |
| REMARK | 3 | | |
| REMARK | 3 | | |
| REMARK | 3 | BULK SOLVENT MODELLING. | |
| REMARK | 3 | METHOD USED : BABINET MODEL WITH MASK | |
| REMARK | 3 | PARAMETERS FOR MASK CALCULATION | |
| REMARK | 3 | VDW PROBE RADIUS | : 1.20 |
| REMARK | 3 | ION PROBE RADIUS | : 0.80 |
| REMARK | 3 | SHRINKAGE RADIUS | : 0.80 |
| REMARK | 3 | | |

| | | |
|---|---|---|
| REMARK | 3 | OTHER REFINEMENT REMARKS: |
| REMARK | 3 | HYDROGENS HAVE BEEN ADDED IN THE RIDING POSITIONS |
| REMARK | 3 | |

| | |
|---|---|
| CRYST1 | 51.105 58.097 61.601 90.00 90.00 90.00 P 21 21 21 |

| | |
|---|---|
| SCALE1 | 0.019568 0.000000 0.000000 0.00000 |
| SCALE2 | 0.000000 0.017213 0.000000 0.00000 |
| SCALE3 | 0.000000 0.000000 0.016234 0.00000 |
| TER | |

| | | | | | | | | | | | |
|---|---|---|---|---|---|---|---|---|---|---|---|
| ATOM | 1 | N | PRO | A | 1 | 12.390 | 14.243 | 29.698 | 1.00 | 29.45 | N |
| ATOM | 2 | CA | PRO | A | 1 | 13.114 | 13.692 | 28.555 | 1.00 | 29.42 | C |
| ATOM | 4 | CB | PRO | A | 1 | 12.296 | 14.166 | 27.350 | 1.00 | 29.72 | C |
| ATOM | 7 | CG | PRO | A | 1 | 10.950 | 14.441 | 27.865 | 1.00 | 29.62 | C |
| ATOM | 10 | CD | PRO | A | 1 | 11.086 | 14.813 | 29.314 | 1.00 | 29.70 | C |
| ATOM | 13 | C | PRO | A | 1 | 14.527 | 14.251 | 28.451 | 1.00 | 28.94 | C |
| ATOM | 14 | O | PRO | A | 1 | 14.747 | 15.422 | 28.746 | 1.00 | 28.90 | O |
| ATOM | 17 | N | GLN | A | 2 | 15.476 | 13.403 | 28.077 | 1.00 | 28.55 | N |
| ATOM | 18 | CA | GLN | A | 2 | 16.792 | 13.854 | 27.668 | 1.00 | 28.32 | C |
| ATOM | 20 | CB | GLN | A | 2 | 17.880 | 13.015 | 28.341 | 1.00 | 28.46 | C |
| ATOM | 23 | CG | GLN | A | 2 | 19.302 | 13.358 | 27.888 | 1.00 | 29.12 | C |
| ATOM | 26 | CD | GLN | A | 2 | 20.366 | 12.827 | 28.831 | 1.00 | 29.77 | C |
| ATOM | 27 | OE1 | GLN | A | 2 | 21.128 | 11.920 | 28.484 | 1.00 | 32.12 | O |
| ATOM | 28 | NE2 | GLN | A | 2 | 20.417 | 13.382 | 30.035 | 1.00 | 29.29 | N |
| ATOM | 31 | C | GLN | A | 2 | 16.875 | 13.721 | 26.149 | 1.00 | 27.79 | C |
| ATOM | 32 | O | GLN | A | 2 | 16.476 | 12.701 | 25.596 | 1.00 | 27.87 | O |
| ATOM | 34 | N | ILE | A | 3 | 17.352 | 14.770 | 25.482 | 1.00 | 26.67 | N |
| ATOM | 35 | CA | ILE | A | 3 | 17.476 | 14.773 | 24.031 | 1.00 | 26.88 | C |
| ATOM | 37 | CB | ILE | A | 3 | 16.583 | 15.881 | 23.393 | 1.00 | 26.43 | C |
| ATOM | 39 | CG1 | ILE | A | 3 | 15.107 | 15.530 | 23.585 | 1.00 | 27.95 | C |
| ATOM | 42 | CD1 | ILE | A | 3 | 14.136 | 16.616 | 23.175 | 1.00 | 27.70 | C |
| ATOM | 46 | CG2 | ILE | A | 3 | 16.910 | 16.059 | 21.910 | 1.00 | 27.19 | C |
| ATOM | 50 | C | ILE | A | 3 | 18.951 | 14.991 | 23.699 | 1.00 | 26.27 | C |
| ATOM | 51 | O | ILE | A | 3 | 19.532 | 15.998 | 24.097 | 1.00 | 24.78 | O |
| ATOM | 53 | N | THR | A | 4 | 19.576 | 14.029 | 23.023 | 1.00 | 26.54 | N |
| ATOM | 54 | CA | THR | A | 4 | 20.974 | 14.208 | 22.608 | 1.00 | 26.87 | C |
| ATOM | 56 | CB | THR | A | 4 | 21.730 | 12.866 | 22.417 | 1.00 | 27.08 | C |
| ATOM | 58 | OG1 | THR | A | 4 | 21.068 | 12.071 | 21.429 | 1.00 | 28.82 | O |
| ATOM | 60 | CG2 | THR | A | 4 | 21.818 | 12.078 | 23.747 | 1.00 | 28.45 | C |
| ATOM | 64 | C | THR | A | 4 | 21.019 | 15.038 | 21.318 | 1.00 | 26.64 | C |
| ATOM | 65 | O | THR | A | 4 | 19.977 | 15.335 | 20.723 | 1.00 | 25.96 | O |
| ATOM | 67 | N | LEU | A | 5 | 22.230 | 15.404 | 20.899 | 1.00 | 26.46 | N |
| ATOM | 68 | CA | LEU | A | 5 | 22.431 | 16.345 | 19.798 | 1.00 | 27.08 | C |
| ATOM | 70 | CB | LEU | A | 5 | 23.126 | 17.606 | 20.320 | 1.00 | 26.37 | C |
| ATOM | 73 | CG | LEU | A | 5 | 22.360 | 18.297 | 21.455 | 1.00 | 26.38 | C |
| ATOM | 75 | CD1 | LEU | A | 5 | 23.176 | 19.446 | 22.061 | 1.00 | 25.84 | C |
| ATOM | 79 | CD2 | LEU | A | 5 | 20.996 | 18.791 | 20.978 | 1.00 | 27.07 | C |
| ATOM | 83 | C | LEU | A | 5 | 23.228 | 15.712 | 18.654 | 1.00 | 27.06 | C |
| ATOM | 84 | O | LEU | A | 5 | 23.814 | 16.398 | 17.821 | 1.00 | 26.72 | O |
| ATOM | 86 | N | TRP | A | 6 | 23.201 | 14.389 | 18.602 | 1.00 | 27.60 | N |
| ATOM | 87 | CA | TRP | A | 6 | 23.717 | 13.636 | 17.465 | 1.00 | 27.72 | C |
| ATOM | 89 | CB | TRP | A | 6 | 23.529 | 12.153 | 17.722 | 1.00 | 28.18 | C |
| ATOM | 92 | CG | TRP | A | 6 | 24.396 | 11.566 | 18.788 | 1.00 | 27.62 | C |
| ATOM | 93 | CD1 | TRP | A | 6 | 23.974 | 10.910 | 19.904 | 1.00 | 27.94 | C |
| ATOM | 95 | NE1 | TRP | A | 6 | 25.047 | 10.435 | 20.614 | 1.00 | 27.31 | N |
| ATOM | 97 | CE2 | TRP | A | 6 | 26.200 | 10.795 | 19.969 | 1.00 | 26.91 | C |
| ATOM | 98 | CD2 | TRP | A | 6 | 25.826 | 11.508 | 18.804 | 1.00 | 26.00 | C |
| ATOM | 99 | CE3 | TRP | A | 6 | 26.828 | 11.991 | 17.959 | 1.00 | 26.28 | C |
| ATOM | 101 | CZ3 | TRP | A | 6 | 28.158 | 11.735 | 18.289 | 1.00 | 27.25 | C |
| ATOM | 103 | CH2 | TRP | A | 6 | 28.497 | 11.021 | 19.446 | 1.00 | 28.03 | C |
| ATOM | 105 | CZ2 | TRP | A | 6 | 27.535 | 10.543 | 20.299 | 1.00 | 28.41 | C |
| ATOM | 107 | C | TRP | A | 6 | 22.974 | 13.979 | 16.178 | 1.00 | 27.82 | C |
| ATOM | 108 | O | TRP | A | 6 | 23.565 | 13.996 | 15.095 | 1.00 | 27.51 | O |
| ATOM | 110 | N | LYS | A | 7 | 21.670 | 14.211 | 16.318 | 1.00 | 26.96 | N |
| ATOM | 111 | CA | LYS | A | 7 | 20.788 | 14.673 | 15.235 | 1.00 | 26.77 | C |
| ATOM | 113 | CB | LYS | A | 7 | 19.710 | 13.621 | 14.955 | 1.00 | 27.12 | C |
| ATOM | 120 | C | LYS | A | 7 | 20.093 | 15.983 | 15.643 | 1.00 | 26.58 | C |
| ATOM | 121 | O | LYS | A | 7 | 20.148 | 16.379 | 16.806 | 1.00 | 26.46 | O |
| ATOM | 123 | N | ARG | A | 8 | 19.459 | 16.655 | 14.685 | 1.00 | 25.92 | N |
| ATOM | 124 | CA | ARG | A | 8 | 18.712 | 17.882 | 14.952 | 1.00 | 26.58 | C |
| ATOM | 126 | CB | ARG | A | 8 | 18.028 | 18.396 | 13.670 | 1.00 | 25.99 | C |
| ATOM | 129 | CG | ARG | A | 8 | 18.997 | 18.941 | 12.626 | 1.00 | 28.93 | C |
| ATOM | 132 | CD | ARG | A | 8 | 18.273 | 19.481 | 11.387 | 1.00 | 28.36 | C |
| ATOM | 135 | NE | ARG | A | 8 | 19.205 | 19.985 | 10.375 | 1.00 | 29.94 | N |
| ATOM | 137 | CZ | ARG | A | 8 | 18.777 | 19.991 | 9.055 | 0.00 | 20.00 | C |
| ATOM | 138 | NH1 | ARG | A | 8 | 17.521 | 20.374 | 8.849 | 0.00 | 20.00 | N |
| ATOM | 141 | NH2 | ARG | A | 8 | 19.638 | 20.013 | 8.044 | 0.00 | 20.00 | N |
| ATOM | 144 | C | ARG | A | 8 | 17.659 | 17.563 | 16.008 | 1.00 | 26.04 | C |
| ATOM | 145 | O | ARG | A | 8 | 16.962 | 16.557 | 15.895 | 1.00 | 25.49 | O |
| ATOM | 147 | N | PRO | A | 9 | 17.577 | 18.375 | 17.071 | 1.00 | 25.99 | N |
| ATOM | 148 | CA | PRO | A | 9 | 16.579 | 18.082 | 18.099 | 1.00 | 26.96 | C |
| ATOM | 150 | CB | PRO | A | 9 | 17.070 | 18.909 | 19.282 | 1.00 | 26.70 | C |
| ATOM | 153 | CG | PRO | A | 9 | 17.727 | 20.095 | 18.629 | 1.00 | 27.09 | C |
| ATOM | 156 | CD | PRO | A | 9 | 18.405 | 19.543 | 17.430 | 1.00 | 26.77 | C |
| ATOM | 159 | C | PRO | A | 9 | 15.166 | 18.507 | 17.667 | 1.00 | 26.77 | C |
| ATOM | 160 | O | PRO | A | 9 | 14.669 | 19.539 | 18.097 | 1.00 | 24.91 | O |
| ATOM | 161 | N | LEU | A | 10 | 14.537 | 17.677 | 16.843 | 1.00 | 26.66 | N |
| ATOM | 162 | CA | LEU | A | 10 | 13.191 | 17.944 | 16.353 | 1.00 | 27.51 | C |
| ATOM | 164 | CB | LEU | A | 10 | 13.017 | 17.441 | 14.922 | 1.00 | 27.86 | C |
| ATOM | 167 | CG | LEU | A | 10 | 13.879 | 18.136 | 13.862 | 1.00 | 28.11 | C |
| ATOM | 169 | CD1 | LEU | A | 10 | 13.927 | 17.303 | 12.583 | 1.00 | 30.30 | C |
| ATOM | 173 | CD2 | LEU | A | 10 | 13.342 | 19.535 | 13.585 | 1.00 | 28.94 | C |
| ATOM | 177 | C | LEU | A | 10 | 12.176 | 17.279 | 17.265 | 1.00 | 27.30 | C |
| ATOM | 178 | O | LEU | A | 10 | 12.371 | 16.156 | 17.719 | 1.00 | 26.55 | O |
| ATOM | 180 | N | VAL | A | 11 | 11.094 | 17.993 | 17.543 | 1.00 | 27.04 | N |
| ATOM | 181 | CA | VAL | A | 11 | 10.037 | 17.464 | 18.385 | 1.00 | 27.13 | C |
| ATOM | 183 | CB | VAL | A | 11 | 10.084 | 18.053 | 19.802 | 1.00 | 26.92 | C |
| ATOM | 185 | CG1 | VAL | A | 11 | 11.426 | 17.752 | 20.469 | 1.00 | 27.67 | C |
| ATOM | 189 | CG2 | VAL | A | 11 | 9.810 | 19.548 | 19.773 | 1.00 | 27.81 | C |
| ATOM | 193 | C | VAL | A | 11 | 8.703 | 17.818 | 17.769 | 1.00 | 27.14 | C |
| ATOM | 194 | O | VAL | A | 11 | 8.623 | 18.644 | 16.858 | 1.00 | 27.19 | O |
| ATOM | 196 | N | THR | A | 12 | 7.652 | 17.222 | 18.314 | 1.00 | 26.58 | N |
| ATOM | 197 | CA | THR | A | 12 | 6.302 | 17.545 | 17.901 | 1.00 | 26.62 | C |
| ATOM | 199 | CB | THR | A | 12 | 5.359 | 16.358 | 18.133 | 1.00 | 26.22 | C |
| ATOM | 201 | OG1 | THR | A | 12 | 5.689 | 15.328 | 17.201 | 1.00 | 26.52 | O |
| ATOM | 203 | CG2 | THR | A | 12 | 3.923 | 16.771 | 17.941 | 1.00 | 26.97 | C |
| ATOM | 207 | C | THR | A | 12 | 5.779 | 18.746 | 18.666 | 1.00 | 26.09 | C |
| ATOM | 208 | O | THR | A | 12 | 5.902 | 18.814 | 19.889 | 1.00 | 26.70 | O |
| ATOM | 210 | N | ILE | A | 13 | 5.204 | 19.690 | 17.929 | 1.00 | 25.72 | N |
| ATOM | 211 | CA | ILE | A | 13 | 4.463 | 20.778 | 18.539 | 1.00 | 25.07 | C |
| ATOM | 213 | CB | ILE | A | 13 | 5.136 | 22.142 | 18.333 | 1.00 | 24.92 | C |
| ATOM | 215 | CG1 | ILE | A | 13 | 5.118 | 22.555 | 16.858 | 1.00 | 25.51 | C |
| ATOM | 218 | CD1 | ILE | A | 13 | 5.211 | 24.041 | 16.652 | 1.00 | 25.29 | C |
| ATOM | 222 | CG2 | ILE | A | 13 | 6.575 | 22.131 | 18.855 | 1.00 | 25.74 | C |
| ATOM | 226 | C | ILE | A | 13 | 3.038 | 20.796 | 17.994 | 1.00 | 25.18 | C |
| ATOM | 227 | O | ILE | A | 13 | 2.783 | 20.386 | 16.859 | 1.00 | 23.22 | O |
| ATOM | 229 | N | ARG | A | 14 | 2.114 | 21.237 | 18.838 | 1.00 | 25.21 | N |
| ATOM | 230 | CA | ARG | A | 14 | 0.744 | 21.478 | 18.418 | 1.00 | 26.78 | C |
| ATOM | 232 | CB | ARG | A | 14 | −0.210 | 20.500 | 19.113 | 1.00 | 27.07 | C |
| ATOM | 235 | CG | ARG | A | 14 | 0.120 | 19.050 | 18.800 | 1.00 | 28.19 | C |
| ATOM | 238 | CD | ARG | A | 14 | −0.864 | 18.064 | 19.390 | 1.00 | 30.36 | C |
| ATOM | 241 | NE | ARG | A | 14 | −0.351 | 16.706 | 19.238 | 1.00 | 33.10 | N |
| ATOM | 243 | CZ | ARG | A | 14 | 0.541 | 16.140 | 20.047 | 1.00 | 35.30 | C |
| ATOM | 244 | NH1 | ARG | A | 14 | 1.007 | 16.785 | 21.112 | 1.00 | 38.14 | N |
| ATOM | 247 | NH2 | ARG | A | 14 | 0.958 | 14.905 | 19.803 | 1.00 | 35.83 | N |
| ATOM | 250 | C | ARG | A | 14 | 0.343 | 22.918 | 18.727 | 1.00 | 26.31 | C |
| ATOM | 251 | O | ARG | A | 14 | 0.552 | 23.411 | 19.839 | 1.00 | 25.34 | O |
| ATOM | 253 | N | ILE | A | 15 | −0.227 | 23.587 | 17.731 | 1.00 | 26.62 | N |
| ATOM | 254 | CA | ILE | A | 15 | −0.751 | 24.922 | 17.928 | 1.00 | 27.66 | C |
| ATOM | 256 | CB | ILE | A | 15 | 0.285 | 26.001 | 17.529 | 1.00 | 28.36 | C |
| ATOM | 258 | CG1 | ILE | A | 15 | −0.389 | 27.345 | 17.245 | 1.00 | 28.75 | C |
| ATOM | 261 | CD1 | ILE | A | 15 | 0.628 | 28.467 | 17.191 | 1.00 | 29.58 | C |
| ATOM | 265 | CG2 | ILE | A | 15 | 1.141 | 25.547 | 16.354 | 1.00 | 29.70 | C |
| ATOM | 269 | C | ILE | A | 15 | −2.080 | 25.055 | 17.204 | 1.00 | 27.56 | C |
| ATOM | 270 | O | ILE | A | 15 | −2.191 | 24.778 | 16.007 | 1.00 | 27.20 | O |
| ATOM | 272 | N | GLY | A | 16 | −3.109 | 25.369 | 17.986 | 1.00 | 27.45 | N |
| ATOM | 273 | CA | GLY | A | 16 | −4.474 | 25.456 | 17.501 | 1.00 | 27.46 | C |
| ATOM | 276 | C | GLY | A | 16 | −4.905 | 24.293 | 16.631 | 1.00 | 27.12 | C |
| ATOM | 277 | O | GLY | A | 16 | −5.592 | 24.497 | 15.628 | 1.00 | 28.00 | O |
| ATOM | 279 | N | GLY | A | 17 | −4.511 | 23.077 | 16.993 | 1.00 | 26.26 | N |
| ATOM | 280 | CA | GLY | A | 17 | −4.886 | 21.908 | 16.197 | 1.00 | 26.89 | C |
| ATOM | 283 | C | GLY | A | 17 | −4.050 | 21.636 | 14.947 | 1.00 | 26.70 | C |
| ATOM | 284 | O | GLY | A | 17 | −4.320 | 20.688 | 14.213 | 1.00 | 26.88 | O |
| ATOM | 286 | N | GLN | A | 18 | −3.054 | 22.471 | 14.681 | 1.00 | 26.30 | N |
| ATOM | 287 | CA | GLN | A | 18 | −2.072 | 22.164 | 13.654 | 1.00 | 26.29 | C |
| ATOM | 289 | CB | GLN | A | 18 | −1.552 | 23.444 | 12.997 | 1.00 | 26.40 | C |
| ATOM | 296 | C | GLN | A | 18 | −0.942 | 21.422 | 14.351 | 1.00 | 26.28 | C |
| ATOM | 297 | O | GLN | A | 18 | −0.527 | 21.797 | 15.437 | 1.00 | 25.86 | O |
| ATOM | 299 | N | LEU | A | 19 | −0.491 | 20.330 | 13.749 | 1.00 | 26.69 | N |
| ATOM | 300 | CA | LEU | A | 19 | 0.575 | 19.527 | 14.324 | 1.00 | 26.69 | C |
| ATOM | 302 | CB | LEU | A | 19 | 0.151 | 18.059 | 14.380 | 1.00 | 26.79 | C |
| ATOM | 305 | CG | LEU | A | 19 | 1.050 | 17.090 | 15.155 | 1.00 | 27.05 | C |
| ATOM | 307 | CD1 | LEU | A | 19 | 1.242 | 16.942 | 15.971 | 0.00 | 20.00 | C |
| ATOM | 311 | CD2 | LEU | A | 19 | 0.369 | 15.556 | 14.087 | 0.00 | 20.00 | C |
| ATOM | 315 | C | LEU | A | 19 | 1.782 | 19.732 | 13.425 | 1.00 | 26.98 | C |
| ATOM | 316 | O | LEU | A | 19 | 1.690 | 19.555 | 12.213 | 1.00 | 26.85 | O |
| ATOM | 318 | N | LYS | A | 20 | 2.882 | 20.186 | 14.017 | 1.00 | 27.55 | N |
| ATOM | 319 | CA | LYS | A | 20 | 4.089 | 20.504 | 13.281 | 1.00 | 28.60 | C |
| ATOM | 321 | CB | LYS | A | 20 | 4.215 | 22.014 | 13.073 | 1.00 | 29.24 | C |
| ATOM | 324 | CG | LYS | A | 20 | 3.208 | 22.590 | 12.081 | 1.00 | 30.69 | C |
| ATOM | 327 | CD | LYS | A | 20 | 2.786 | 23.997 | 12.475 | 1.00 | 32.00 | C |
| ATOM | 330 | CE | LYS | A | 20 | 1.863 | 24.604 | 11.442 | 1.00 | 31.64 | C |
| ATOM | 333 | NZ | LYS | A | 20 | 2.635 | 25.234 | 10.332 | 1.00 | 35.13 | N |
| ATOM | 337 | C | LYS | A | 20 | 5.332 | 19.980 | 13.992 | 1.00 | 28.58 | C |
| ATOM | 338 | O | LYS | A | 20 | 5.295 | 19.576 | 15.156 | 1.00 | 29.08 | O |
| ATOM | 340 | N | GLU | A | 21 | 6.423 | 19.970 | 13.241 | 1.00 | 27.83 | N |
| ATOM | 341 | CA | GLU | A | 21 | 7.712 | 19.529 | 13.720 | 1.00 | 27.49 | C |
| ATOM | 343 | CB | GLU | A | 21 | 8.342 | 18.617 | 12.659 | 1.00 | 27.47 | C |
| ATOM | 346 | CG | GLU | A | 21 | 9.855 | 18.563 | 12.621 | 1.00 | 28.86 | C |
| ATOM | 349 | CD | GLU | A | 21 | 10.350 | 17.486 | 11.681 | 1.00 | 29.20 | C |
| ATOM | 350 | OE1 | GLU | A | 21 | 11.139 | 17.803 | 10.764 | 1.00 | 33.12 | O |
| ATOM | 351 | OE2 | GLU | A | 21 | 9.914 | 16.324 | 11.833 | 1.00 | 32.07 | O |
| ATOM | 352 | C | GLU | A | 21 | 8.525 | 20.803 | 13.956 | 1.00 | 27.04 | C |
| ATOM | 353 | O | GLU | A | 21 | 8.511 | 21.710 | 13.126 | 1.00 | 26.24 | O |
| ATOM | 355 | N | ALA | A | 22 | 9.192 | 20.883 | 15.100 | 1.00 | 26.07 | N |
| ATOM | 356 | CA | ALA | A | 22 | 10.002 | 22.052 | 15.415 | 1.00 | 26.91 | C |
| ATOM | 358 | CB | ALA | A | 22 | 9.222 | 22.992 | 16.349 | 1.00 | 26.51 | C |
| ATOM | 362 | C | ALA | A | 22 | 11.359 | 21.676 | 16.026 | 1.00 | 26.80 | C |
| ATOM | 363 | O | ALA | A | 22 | 11.499 | 20.646 | 16.678 | 1.00 | 27.59 | O |
| ATOM | 365 | N | LEU | A | 23 | 12.345 | 22.525 | 15.760 | 1.00 | 26.80 | N |
| ATOM | 366 | CA | LEU | A | 23 | 13.702 | 22.437 | 16.299 | 1.00 | 26.85 | C |
| ATOM | 368 | CB | LEU | A | 23 | 14.642 | 23.172 | 15.331 | 1.00 | 26.28 | C |
| ATOM | 371 | CG | LEU | A | 23 | 16.150 | 22.997 | 15.457 | 1.00 | 27.03 | C |
| ATOM | 373 | CD1 | LEU | A | 23 | 16.518 | 21.531 | 15.271 | 1.00 | 26.39 | C |
| ATOM | 377 | CD2 | LEU | A | 23 | 16.855 | 23.850 | 14.400 | 1.00 | 27.38 | C |
| ATOM | 381 | C | LEU | A | 23 | 13.800 | 23.104 | 17.667 | 1.00 | 25.73 | C |
| ATOM | 382 | O | LEU | A | 23 | 13.483 | 24.277 | 17.800 | 1.00 | 27.74 | O |
| ATOM | 384 | N | LEU | A | 24 | 14.286 | 22.391 | 18.673 | 1.00 | 26.44 | N |
| ATOM | 385 | CA | LEU | A | 24 | 14.579 | 23.030 | 19.960 | 1.00 | 26.95 | C |
| ATOM | 387 | CB | LEU | A | 24 | 14.616 | 22.003 | 21.099 | 1.00 | 26.71 | C |
| ATOM | 390 | CG | LEU | A | 24 | 13.351 | 21.160 | 21.254 | 1.00 | 27.86 | C |
| ATOM | 392 | CD1 | LEU | A | 24 | 13.604 | 20.058 | 22.231 | 1.00 | 27.07 | C |
| ATOM | 396 | CD2 | LEU | A | 24 | 12.158 | 22.000 | 21.694 | 1.00 | 28.97 | C |
| ATOM | 400 | C | LEU | A | 24 | 15.917 | 23.764 | 19.825 | 1.00 | 26.55 | C |
| ATOM | 401 | O | LEU | A | 24 | 16.943 | 23.139 | 19.583 | 1.00 | 28.65 | O |
| ATOM | 403 | N | ASP | A | 25 | 15.887 | 25.085 | 19.985 | 1.00 | 26.47 | N |
| ATOM | 404 | CA | ASP | A | 25 | 16.932 | 25.975 | 19.467 | 1.00 | 26.16 | C |
| ATOM | 406 | CB | ASP | A | 25 | 16.420 | 26.698 | 18.203 | 1.00 | 25.46 | C |
| ATOM | 409 | CG | ASP | A | 25 | 17.498 | 27.492 | 17.480 | 1.00 | 26.98 | C |
| ATOM | 410 | OD1 | ASP | A | 25 | 17.248 | 27.875 | 16.320 | 1.00 | 27.89 | O |
| ATOM | 411 | OD2 | ASP | A | 25 | 18.585 | 27.741 | 18.049 | 1.00 | 28.65 | O |
| ATOM | 412 | C | ASP | A | 25 | 17.345 | 26.968 | 20.553 | 1.00 | 26.06 | C |
| ATOM | 413 | O | ASP | A | 25 | 16.816 | 28.077 | 20.649 | 1.00 | 26.16 | O |
| ATOM | 415 | N | THR | A | 26 | 18.340 | 26.564 | 21.338 | 1.00 | 26.23 | N |
| ATOM | 416 | CA | THR | A | 26 | 18.821 | 27.329 | 22.486 | 1.00 | 25.48 | C |
| ATOM | 418 | CB | THR | A | 26 | 19.813 | 26.482 | 23.340 | 1.00 | 25.53 | C |
| ATOM | 420 | OG1 | THR | A | 26 | 20.929 | 26.070 | 22.539 | 1.00 | 25.69 | O |
| ATOM | 422 | CG2 | THR | A | 26 | 19.139 | 25.250 | 23.896 | 1.00 | 23.68 | C |
| ATOM | 426 | C | THR | A | 26 | 19.513 | 28.626 | 22.078 | 1.00 | 26.08 | C |
| ATOM | 427 | O | THR | A | 26 | 19.587 | 29.577 | 22.877 | 1.00 | 25.09 | N |
| ATOM | 429 | N | GLY | A | 27 | 19.986 | 28.668 | 20.832 | 1.00 | 25.31 | N |
| ATOM | 430 | CA | GLY | A | 27 | 20.533 | 29.882 | 20.231 | 1.00 | 25.80 | C |
| ATOM | 433 | C | GLY | A | 27 | 19.515 | 30.980 | 19.946 | 1.00 | 25.71 | C |
| ATOM | 434 | O | GLY | A | 27 | 19.849 | 32.162 | 20.006 | 1.00 | 26.96 | O |
| ATOM | 436 | N | ALA | A | 28 | 18.274 | 30.602 | 19.651 | 1.00 | 25.52 | N |
| ATOM | 437 | CA | ALA | A | 28 | 17.256 | 31.557 | 19.259 | 1.00 | 25.59 | C |
| ATOM | 439 | CB | ALA | A | 28 | 16.163 | 30.855 | 18.466 | 1.00 | 26.07 | C |
| ATOM | 443 | C | ALA | A | 28 | 16.640 | 32.209 | 20.487 | 1.00 | 26.21 | C |
| ATOM | 444 | O | ALA | A | 28 | 16.191 | 31.501 | 21.396 | 1.00 | 24.21 | O |
| ATOM | 446 | N | ASP | A | 29 | 16.591 | 33.545 | 20.479 | 1.00 | 25.80 | N |
| ATOM | 447 | CA | ASP | A | 29 | 15.962 | 34.319 | 21.541 | 1.00 | 26.65 | C |
| ATOM | 449 | CB | ASP | A | 29 | 16.148 | 35.820 | 21.304 | 1.00 | 26.71 | C |
| ATOM | 452 | CG | ASP | A | 29 | 17.591 | 36.262 | 21.407 | 1.00 | 26.70 | C |
| ATOM | 453 | OD1 | ASP | A | 29 | 18.378 | 35.644 | 22.161 | 1.00 | 21.74 | O |
| ATOM | 454 | OD2 | ASP | A | 29 | 17.921 | 37.264 | 20.741 | 1.00 | 22.18 | O |
| ATOM | 455 | C | ASP | A | 29 | 14.466 | 34.030 | 21.572 | 1.00 | 27.08 | C |
| ATOM | 456 | O | ASP | A | 29 | 13.864 | 33.867 | 22.636 | 1.00 | 26.82 | O |
| ATOM | 458 | N | ASP | A | 30 | 13.909 | 33.965 | 20.368 | 1.00 | 27.83 | N |
| ATOM | 459 | CA | ASP | A | 30 | 12.481 | 33.938 | 20.107 | 1.00 | 27.64 | C |
| ATOM | 461 | CB | ASP | A | 30 | 12.093 | 35.154 | 19.273 | 1.00 | 28.08 | C |
| ATOM | 464 | CG | ASP | A | 30 | 12.518 | 36.458 | 19.910 | 1.00 | 30.71 | C |
| ATOM | 465 | OD1 | ASP | A | 30 | 12.227 | 36.639 | 21.118 | 1.00 | 32.56 | O |
| ATOM | 466 | OD2 | ASP | A | 30 | 13.154 | 37.284 | 19.208 | 1.00 | 31.25 | O |
| ATOM | 467 | C | ASP | A | 30 | 12.146 | 32.690 | 19.305 | 1.00 | 26.99 | C |
| ATOM | 468 | O | ASP | A | 30 | 13.018 | 32.033 | 18.735 | 1.00 | 27.43 | O |
| ATOM | 470 | N | THR | A | 31 | 10.858 | 32.407 | 19.240 | 1.00 | 26.97 | N |
| ATOM | 471 | CA | THR | A | 31 | 10.325 | 31.258 | 18.535 | 1.00 | 26.14 | C |
| ATOM | 473 | CB | THR | A | 31 | 9.176 | 30.644 | 19.373 | 1.00 | 26.76 | C |
| ATOM | 475 | OG1 | THR | A | 31 | 9.736 | 30.017 | 20.533 | 1.00 | 26.19 | O |
| ATOM | 477 | CG2 | THR | A | 31 | 8.342 | 29.643 | 18.586 | 1.00 | 25.32 | C |
| ATOM | 481 | C | THR | A | 31 | 9.835 | 31.764 | 17.185 | 1.00 | 25.78 | C |
| ATOM | 482 | O | THR | A | 31 | 9.044 | 32.714 | 17.106 | 1.00 | 25.34 | O |
| ATOM | 484 | N | VAL | A | 32 | 10.296 | 31.113 | 16.127 | 1.00 | 25.58 | N |
| ATOM | 485 | CA | VAL | A | 32 | 9.992 | 31.526 | 14.768 | 1.00 | 25.66 | C |
| ATOM | 487 | CB | VAL | A | 32 | 11.266 | 31.939 | 13.994 | 1.00 | 25.97 | C |
| ATOM | 489 | CG1 | VAL | A | 32 | 10.900 | 32.507 | 12.632 | 1.00 | 25.78 | C |
| ATOM | 493 | CG2 | VAL | A | 32 | 12.065 | 32.934 | 14.800 | 1.00 | 26.31 | C |
| ATOM | 497 | C | VAL | A | 32 | 9.350 | 30.364 | 14.047 | 1.00 | 26.11 | C |
| ATOM | 498 | O | VAL | A | 32 | 9.983 | 29.316 | 13.837 | 1.00 | 24.86 | O |
| ATOM | 500 | N | LEU | A | 33 | 8.092 | 30.557 | 13.669 | 1.00 | 26.01 | N |
| ATOM | 501 | CA | LEU | A | 33 | 7.328 | 29.510 | 13.019 | 1.00 | 26.89 | C |
| ATOM | 503 | CB | LEU | A | 33 | 5.956 | 29.369 | 13.670 | 1.00 | 26.92 | C |
| ATOM | 506 | CG | LEU | A | 33 | 5.880 | 28.958 | 15.141 | 1.00 | 27.94 | C |
| ATOM | 508 | CD1 | LEU | A | 33 | 4.443 | 28.563 | 15.469 | 1.00 | 28.15 | C |
| ATOM | 512 | CD2 | LEU | A | 33 | 6.827 | 27.829 | 15.452 | 1.00 | 28.57 | C |
| ATOM | 516 | C | LEU | A | 33 | 7.123 | 29.800 | 11.543 | 1.00 | 26.87 | C |
| ATOM | 517 | O | LEU | A | 33 | 6.947 | 30.952 | 11.148 | 1.00 | 27.54 | O |
| ATOM | 519 | N | GLU | A | 34 | 7.108 | 28.731 | 10.752 | 1.00 | 26.80 | N |
| ATOM | 520 | CA | GLU | A | 34 | 6.736 | 28.777 | 9.349 | 1.00 | 27.07 | C |
| ATOM | 522 | CB | GLU | A | 34 | 6.644 | 27.358 | 8.767 | 1.00 | 27.37 | C |
| ATOM | 525 | CG | GLU | A | 34 | 5.469 | 26.530 | 9.317 | 1.00 | 27.40 | C |
| ATOM | 528 | CD | GLU | A | 34 | 5.657 | 25.029 | 9.160 | 1.00 | 27.28 | C |
| ATOM | 529 | OE1 | GLU | A | 34 | 6.382 | 24.597 | 8.239 | 1.00 | 28.26 | O |
| ATOM | 530 | OE2 | GLU | A | 34 | 5.083 | 24.269 | 9.968 | 1.00 | 27.79 | O |
| ATOM | 531 | C | GLU | A | 34 | 5.385 | 29.448 | 9.180 | 1.00 | 27.88 | C |
| ATOM | 532 | O | GLU | A | 34 | 4.583 | 29.549 | 10.127 | 1.00 | 28.02 | O |
| ATOM | 534 | N | GLU | A | 35 | 5.134 | 29.885 | 7.957 | 1.00 | 27.10 | N |
| ATOM | 535 | CA | GLU | A | 35 | 3.934 | 30.634 | 7.662 | 1.00 | 27.81 | C |
| ATOM | 537 | CB | GLU | A | 35 | 3.870 | 30.952 | 6.165 | 1.00 | 28.17 | C |
| ATOM | 540 | CG | GLU | A | 35 | 4.547 | 32.265 | 5.836 | 1.00 | 29.45 | C |
| ATOM | 543 | CD | GLU | A | 35 | 3.857 | 33.424 | 6.516 | 1.00 | 31.61 | C |
| ATOM | 544 | OE1 | GLU | A | 35 | 2.630 | 33.307 | 6.767 | 1.00 | 31.53 | O |
| ATOM | 545 | OE2 | GLU | A | 35 | 4.537 | 34.439 | 6.791 | 1.00 | 33.36 | O |
| ATOM | 546 | C | GLU | A | 35 | 2.667 | 29.922 | 8.124 | 1.00 | 27.51 | C |
| ATOM | 547 | O | GLU | A | 35 | 2.450 | 28.753 | 7.815 | 1.00 | 27.82 | O |
| ATOM | 549 | N | MET | A | 36 | 1.853 | 30.646 | 8.890 | 1.00 | 27.31 | N |
| ATOM | 550 | CA | MET | A | 36 | 0.579 | 30.144 | 9.389 | 1.00 | 27.10 | C |
| ATOM | 552 | CB | MET | A | 36 | 0.805 | 29.159 | 10.535 | 1.00 | 26.71 | C |
| ATOM | 555 | CG | MET | A | 36 | 1.333 | 29.781 | 11.811 | 1.00 | 27.10 | C |
| ATOM | 558 | SD | MET | A | 36 | 1.464 | 28.603 | 13.174 | 1.00 | 27.31 | S |
| ATOM | 559 | CE | MET | A | 36 | 2.744 | 27.526 | 12.542 | 1.00 | 29.56 | C |
| ATOM | 563 | C | MET | A | 36 | −0.316 | 31.295 | 9.844 | 1.00 | 26.74 | C |
| ATOM | 564 | O | MET | A | 36 | 0.164 | 32.327 | 10.319 | 1.00 | 26.70 | O |
| ATOM | 566 | N | ASN | A | 37 | −1.619 | 31.127 | 9.654 | 1.00 | 26.78 | N |
| ATOM | 567 | CA | ASN | A | 37 | −2.596 | 32.049 | 10.206 | 1.00 | 26.74 | C |
| ATOM | 569 | CB | ASN | A | 37 | −3.976 | 31.830 | 9.574 | 1.00 | 27.09 | C |
| ATOM | 572 | CG | ASN | A | 37 | −4.073 | 32.392 | 8.172 | 1.00 | 27.67 | C |
| ATOM | 573 | OD1 | ASN | A | 37 | −3.062 | 32.575 | 7.496 | 1.00 | 30.45 | O |
| ATOM | 574 | ND2 | ASN | A | 37 | −5.294 | 32.680 | 7.730 | 1.00 | 29.73 | N |
| ATOM | 577 | C | ASN | A | 37 | −2.677 | 31.800 | 11.698 | 1.00 | 26.75 | C |
| ATOM | 578 | O | ASN | A | 37 | −2.858 | 30.665 | 12.127 | 1.00 | 26.25 | O |
| ATOM | 580 | N | LEU | A | 38 | −2.487 | 32.851 | 12.483 | 1.00 | 26.80 | N |
| ATOM | 581 | CA | LEU | A | 38 | −2.826 | 32.814 | 13.894 | 1.00 | 26.80 | C |
| ATOM | 583 | CB | LEU | A | 38 | −1.588 | 33.034 | 14.771 | 1.00 | 26.96 | C |
| ATOM | 586 | CG | LEU | A | 38 | −0.514 | 31.940 | 14.724 | 1.00 | 26.64 | C |
| ATOM | 588 | CD1 | LEU | A | 38 | 0.718 | 32.368 | 15.491 | 1.00 | 29.14 | C |
| ATOM | 592 | CD2 | LEU | A | 38 | −1.043 | 30.628 | 15.252 | 1.00 | 26.25 | C |
| ATOM | 596 | C | LEU | A | 38 | −3.847 | 33.909 | 14.145 | 1.00 | 26.69 | C |
| ATOM | 597 | O | LEU | A | 38 | −3.877 | 34.903 | 13.429 | 1.00 | 26.86 | O |
| ATOM | 599 | N | PRO | A | 39 | −4.681 | 33.732 | 15.175 | 1.00 | 26.87 | N |
| ATOM | 600 | CA | PRO | A | 39 | −5.668 | 34.732 | 15.528 | 1.00 | 26.83 | C |
| ATOM | 602 | CB | PRO | A | 39 | −6.606 | 33.999 | 16.490 | 1.00 | 27.16 | C |
| ATOM | 605 | CG | PRO | A | 39 | −5.886 | 32.773 | 16.933 | 1.00 | 27.43 | C |
| ATOM | 608 | CD | PRO | A | 39 | −4.663 | 32.584 | 16.097 | 1.00 | 27.18 | C |
| ATOM | 611 | C | PRO | A | 39 | −5.011 | 35.913 | 16.211 | 1.00 | 26.93 | C |
| ATOM | 612 | O | PRO | A | 39 | −3.904 | 35.787 | 16.724 | 1.00 | 27.59 | O |
| ATOM | 613 | N | GLY | A | 40 | −5.665 | 37.066 | 16.171 | 1.00 | 27.40 | N |
| ATOM | 614 | CA | GLY | A | 40 | −5.229 | 38.215 | 16.953 | 1.00 | 27.49 | C |
| ATOM | 617 | C | GLY | A | 40 | −4.377 | 39.217 | 16.198 | 1.00 | 27.67 | C |
| ATOM | 618 | O | GLY | A | 40 | −3.960 | 38.995 | 15.059 | 1.00 | 27.45 | O |
| ATOM | 620 | N | ARG | A | 41 | −4.132 | 40.341 | 16.857 | 1.00 | 28.06 | N |
| ATOM | 621 | CA | ARG | A | 41 | −3.283 | 41.386 | 16.317 | 1.00 | 27.98 | C |
| ATOM | 623 | CB | ARG | A | 41 | −3.389 | 42.634 | 17.193 | 1.00 | 27.94 | C |
| ATOM | 632 | C | ARG | A | 41 | −1.831 | 40.905 | 16.246 | 1.00 | 28.31 | C |
| ATOM | 633 | O | ARG | A | 41 | −1.353 | 40.193 | 17.134 | 1.00 | 28.33 | O |
| ATOM | 635 | N | TRP | A | 42 | −1.156 | 41.269 | 15.158 | 1.00 | 28.32 | N |
| ATOM | 636 | CA | TRP | A | 42 | 0.287 | 41.107 | 15.041 | 1.00 | 28.22 | C |
| ATOM | 638 | CB | TRP | A | 42 | 0.629 | 40.027 | 14.013 | 1.00 | 28.77 | C |
| ATOM | 641 | CG | TRP | A | 42 | 0.023 | 40.261 | 12.660 | 1.00 | 29.20 | C |
| ATOM | 642 | CD1 | TRP | A | 42 | −1.222 | 39.877 | 12.241 | 1.00 | 30.46 | C |
| ATOM | 644 | NE1 | TRP | A | 42 | −1.427 | 40.269 | 10.941 | 1.00 | 28.61 | N |
| ATOM | 646 | CE2 | TRP | A | 42 | −0.321 | 40.942 | 10.502 | 1.00 | 30.21 | C |
| ATOM | 647 | CD2 | TRP | A | 42 | 0.619 | 40.947 | 11.554 | 1.00 | 29.71 | C |
| ATOM | 648 | CE3 | TRP | A | 42 | 1.841 | 41.592 | 11.358 | 1.00 | 30.09 | C |
| ATOM | 650 | CZ3 | TRP | A | 42 | 2.094 | 42.170 | 10.134 | 1.00 | 29.56 | C |
| ATOM | 652 | CH2 | TRP | A | 42 | 1.151 | 42.128 | 9.102 | 1.00 | 29.71 | C |
| ATOM | 654 | CZ2 | TRP | A | 42 | −0.057 | 41.520 | 9.265 | 1.00 | 29.95 | C |
| ATOM | 656 | C | TRP | A | 42 | 0.941 | 42.427 | 14.638 | 1.00 | 27.95 | C |
| ATOM | 657 | O | TRP | A | 42 | 0.305 | 43.290 | 14.033 | 1.00 | 26.82 | O |
| ATOM | 659 | N | LYS | A | 43 | 2.214 | 42.572 | 14.987 | 1.00 | 28.19 | N |
| ATOM | 660 | CA | LYS | A | 43 | 3.026 | 43.680 | 14.507 | 1.00 | 28.36 | C |
| ATOM | 662 | CB | LYS | A | 43 | 3.412 | 44.608 | 15.652 | 1.00 | 28.44 | C |
| ATOM | 665 | CG | LYS | A | 43 | 4.377 | 44.030 | 16.668 | 1.00 | 28.16 | C |
| ATOM | 668 | CD | LYS | A | 43 | 4.237 | 44.796 | 17.985 | 1.00 | 28.74 | C |
| ATOM | 671 | CE | LYS | A | 43 | 5.477 | 44.690 | 18.864 | 1.00 | 29.03 | C |
| ATOM | 674 | NZ | LYS | A | 43 | 5.167 | 44.981 | 20.304 | 1.00 | 29.69 | N |
| ATOM | 678 | C | LYS | A | 43 | 4.284 | 43.151 | 13.835 | 1.00 | 28.46 | C |
| ATOM | 679 | O | LYS | A | 43 | 4.787 | 42.083 | 14.198 | 1.00 | 28.47 | O |
| ATOM | 681 | N | PRO | A | 44 | 4.778 | 43.886 | 12.832 | 1.00 | 27.95 | N |
| ATOM | 682 | CA | PRO | A | 44 | 6.020 | 43.518 | 12.169 | 1.00 | 27.55 | C |
| ATOM | 684 | CB | PRO | A | 44 | 6.150 | 44.540 | 11.031 | 1.00 | 28.14 | C |
| ATOM | 687 | CG | PRO | A | 44 | 4.902 | 45.292 | 10.973 | 1.00 | 28.47 | C |
| ATOM | 690 | CD | PRO | A | 44 | 4.154 | 45.088 | 12.251 | 1.00 | 28.44 | C |
| ATOM | 693 | C | PRO | A | 44 | 7.235 | 43.590 | 13.096 | 1.00 | 26.79 | C |
| ATOM | 694 | O | PRO | A | 44 | 7.337 | 44.481 | 13.943 | 1.00 | 25.27 | O |
| ATOM | 695 | N | LYS | A | 45 | 8.162 | 42.660 | 12.906 | 1.00 | 26.40 | N |
| ATOM | 696 | CA | LYS | A | 45 | 9.426 | 42.678 | 13.623 | 1.00 | 26.29 | C |
| ATOM | 698 | CB | LYS | A | 45 | 9.352 | 41.721 | 14.817 | 1.00 | 26.55 | C |
| ATOM | 701 | CG | LYS | A | 45 | 10.519 | 41.807 | 15.783 | 1.00 | 26.15 | C |
| ATOM | 704 | CD | LYS | A | 45 | 10.433 | 40.692 | 16.826 | 1.00 | 26.65 | C |
| ATOM | 707 | CE | LYS | A | 45 | 11.362 | 40.959 | 18.003 | 1.00 | 26.91 | C |
| ATOM | 710 | NZ | LYS | A | 45 | 10.826 | 42.037 | 18.880 | 1.00 | 28.28 | N |
| ATOM | 714 | C | LYS | A | 45 | 10.514 | 42.214 | 12.670 | 1.00 | 26.16 | C |
| ATOM | 715 | O | LYS | A | 45 | 10.221 | 41.534 | 11.689 | 1.00 | 25.98 | O |
| ATOM | 717 | N | MET | A | 46 | 11.764 | 42.543 | 12.986 | 1.00 | 25.25 | N |
| ATOM | 718 | CA | MET | A | 46 | 12.904 | 42.040 | 12.235 | 1.00 | 25.87 | C |
| ATOM | 720 | CB | MET | A | 46 | 13.737 | 43.199 | 11.701 | 1.00 | 25.91 | C |
| ATOM | 723 | CG | MET | A | 46 | 12.995 | 44.059 | 10.714 | 1.00 | 26.57 | C |
| ATOM | 726 | SD | MET | A | 46 | 12.930 | 43.239 | 9.128 | 1.00 | 28.21 | S |
| ATOM | 727 | CE | MET | A | 46 | 14.565 | 43.600 | 8.477 | 1.00 | 29.83 | C |
| ATOM | 731 | C | MET | A | 46 | 13.756 | 41.197 | 13.153 | 1.00 | 26.15 | C |
| ATOM | 732 | O | MET | A | 46 | 14.093 | 41.622 | 14.257 | 1.00 | 27.17 | O |
| ATOM | 734 | N | AILE | A | 47 | 14.101 | 39.998 | 12.699 | 0.50 | 26.39 | N |
| ATOM | 735 | N | BILE | A | 47 | 14.090 | 39.992 | 12.703 | 0.50 | 26.41 | N |
| ATOM | 736 | CA | AILE | A | 47 | 14.948 | 39.103 | 13.473 | 0.50 | 26.37 | C |
| ATOM | 737 | CA | BILE | A | 47 | 14.939 | 39.087 | 13.469 | 0.50 | 26.41 | C |
| ATOM | 740 | CB | AILE | A | 47 | 14.187 | 37.841 | 13.934 | 0.50 | 26.78 | C |
| ATOM | 741 | CB | BILE | A | 47 | 14.192 | 37.799 | 13.886 | 0.50 | 26.79 | C |
| ATOM | 744 | CG1 | AILE | A | 47 | 15.015 | 37.063 | 14.955 | 0.50 | 26.35 | C |
| ATOM | 745 | CG1 | BILE | A | 47 | 13.602 | 37.082 | 12.668 | 0.50 | 27.09 | C |
| ATOM | 750 | CD1 | AILE | A | 47 | 14.203 | 36.074 | 15.729 | 0.50 | 27.05 | C |
| ATOM | 751 | CD1 | BILE | A | 47 | 13.081 | 35.705 | 12.975 | 0.50 | 27.28 | C |
| ATOM | 758 | CG2 | AILE | A | 47 | 13.814 | 36.948 | 12.754 | 0.50 | 26.99 | C |
| ATOM | 759 | CG2 | BILE | A | 47 | 13.078 | 38.129 | 14.876 | 0.50 | 26.33 | C |
| ATOM | 766 | C | AILE | A | 47 | 16.171 | 38.729 | 12.649 | 0.50 | 26.39 | C |
| ATOM | 767 | C | BILE | A | 47 | 16.173 | 38.740 | 12.646 | 0.50 | 26.39 | C |
| ATOM | 768 | O | AILE | A | 47 | 16.067 | 38.470 | 11.445 | 0.50 | 25.58 | O |
| ATOM | 769 | O | BILE | A | 47 | 16.078 | 38.511 | 11.435 | 0.50 | 25.57 | O |
| ATOM | 772 | N | GLY | A | 48 | 17.326 | 38.739 | 13.308 | 1.00 | 26.11 | N |
| ATOM | 773 | CA | GLY | A | 48 | 18.606 | 38.472 | 12.654 | 1.00 | 26.89 | C |
| ATOM | 776 | C | GLY | A | 48 | 19.006 | 37.010 | 12.714 | 1.00 | 26.47 | C |
| ATOM | 777 | O | GLY | A | 48 | 18.662 | 36.293 | 13.662 | 1.00 | 25.76 | O |
| ATOM | 779 | N | GLY | A | 49 | 19.693 | 36.576 | 11.661 | 1.00 | 26.24 | N |
| ATOM | 780 | CA | GLY | A | 49 | 20.307 | 35.251 | 11.602 | 1.00 | 26.46 | C |
| ATOM | 783 | C | GLY | A | 49 | 21.663 | 35.361 | 10.946 | 1.00 | 25.02 | C |
| ATOM | 784 | O | GLY | A | 49 | 22.097 | 36.449 | 10.603 | 1.00 | 24.78 | O |
| ATOM | 786 | N | ILE | A | 50 | 22.267 | 34.218 | 10.655 | 1.00 | 24.88 | N |
| ATOM | 787 | CA | ILE | A | 50 | 23.562 | 34.180 | 10.001 | 1.00 | 24.37 | C |
| ATOM | 789 | CB | ILE | A | 50 | 24.070 | 32.731 | 9.915 | 1.00 | 24.56 | C |
| ATOM | 791 | CG1 | ILE | A | 50 | 24.910 | 32.429 | 11.160 | 1.00 | 23.79 | C |
| ATOM | 794 | CD1 | ILE | A | 50 | 25.615 | 31.116 | 11.107 | 1.00 | 23.74 | C |
| ATOM | 798 | CG2 | ILE | A | 50 | 24.888 | 32.496 | 8.673 | 1.00 | 25.42 | C |
| ATOM | 802 | C | ILE | A | 50 | 23.628 | 34.913 | 8.652 | 1.00 | 24.57 | C |
| ATOM | 803 | O | ILE | A | 50 | 24.650 | 35.517 | 8.330 | 1.00 | 25.03 | O |
| ATOM | 805 | N | GLY | A | 51 | 22.554 | 34.901 | 7.877 | 1.00 | 23.65 | N |
| ATOM | 806 | CA | GLY | A | 51 | 22.565 | 35.605 | 6.581 | 1.00 | 24.05 | C |
| ATOM | 809 | C | GLY | A | 51 | 22.135 | 37.070 | 6.554 | 1.00 | 24.17 | C |
| ATOM | 810 | O | GLY | A | 51 | 22.203 | 37.738 | 5.515 | 1.00 | 23.90 | O |
| ATOM | 812 | N | GLY | A | 52 | 21.657 | 37.563 | 7.686 | 1.00 | 23.69 | N |
| ATOM | 813 | CA | GLY | A | 52 | 20.976 | 38.843 | 7.748 | 1.00 | 24.82 | C |
| ATOM | 816 | C | GLY | A | 52 | 19.623 | 38.697 | 8.418 | 1.00 | 25.17 | C |
| ATOM | 817 | O | GLY | A | 52 | 19.398 | 37.767 | 9.196 | 1.00 | 25.11 | O |
| ATOM | 819 | N | PHE | A | 53 | 18.705 | 39.605 | 8.102 | 1.00 | 24.79 | N |
| ATOM | 820 | CA | PHE | A | 53 | 17.455 | 39.690 | 8.836 | 1.00 | 24.70 | C |
| ATOM | 822 | CB | PHE | A | 53 | 17.204 | 41.127 | 9.282 | 1.00 | 24.08 | C |
| ATOM | 825 | CG | PHE | A | 53 | 17.981 | 41.527 | 10.503 | 1.00 | 20.96 | C |
| ATOM | 826 | CD1 | PHE | A | 53 | 19.313 | 41.886 | 10.400 | 1.00 | 22.68 | C |
| ATOM | 828 | CE1 | PHE | A | 53 | 20.022 | 42.277 | 11.510 | 1.00 | 20.93 | C |
| ATOM | 830 | CZ | PHE | A | 53 | 19.412 | 42.314 | 12.752 | 1.00 | 21.92 | C |
| ATOM | 832 | CE2 | PHE | A | 53 | 18.077 | 41.989 | 12.873 | 1.00 | 22.15 | C |
| ATOM | 834 | CD2 | PHE | A | 53 | 17.361 | 41.604 | 11.739 | 1.00 | 20.68 | C |
| ATOM | 836 | C | PHE | A | 53 | 16.295 | 39.212 | 7.982 | 1.00 | 26.28 | C |
| ATOM | 837 | O | PHE | A | 53 | 16.335 | 39.331 | 6.762 | 1.00 | 27.74 | O |
| ATOM | 839 | N | ILE | A | 54 | 15.279 | 38.647 | 8.620 | 1.00 | 26.66 | N |
| ATOM | 840 | CA | ILE | A | 54 | 13.992 | 38.482 | 7.965 | 1.00 | 27.93 | C |
| ATOM | 842 | CB | ILE | A | 54 | 13.567 | 37.016 | 7.790 | 1.00 | 27.50 | C |
| ATOM | 844 | CG1 | ILE | A | 54 | 13.474 | 36.278 | 9.130 | 1.00 | 28.01 | C |
| ATOM | 847 | CD1 | ILE | A | 54 | 12.614 | 35.033 | 9.032 | 1.00 | 28.46 | C |
| ATOM | 851 | CG2 | ILE | A | 54 | 14.489 | 36.318 | 6.826 | 1.00 | 29.10 | C |
| ATOM | 855 | C | ILE | A | 54 | 12.908 | 39.207 | 8.738 | 1.00 | 27.40 | C |
| ATOM | 856 | O | ILE | A | 54 | 12.996 | 39.359 | 9.956 | 1.00 | 28.17 | O |
| ATOM | 858 | N | LYS | A | 55 | 11.881 | 39.624 | 8.013 | 1.00 | 27.09 | N |
| ATOM | 859 | CA | LYS | A | 55 | 10.728 | 40.255 | 8.618 | 1.00 | 26.59 | C |
| ATOM | 861 | CB | LYS | A | 55 | 10.076 | 41.238 | 7.651 | 1.00 | 26.83 | C |
| ATOM | 864 | CG | LYS | A | 55 | 9.003 | 42.106 | 8.302 | 1.00 | 26.28 | C |
| ATOM | 867 | CD | LYS | A | 55 | 8.412 | 43.120 | 7.320 | 1.00 | 28.27 | C |
| ATOM | 870 | CE | LYS | A | 55 | 7.107 | 43.477 | 7.118 | 0.00 | 20.00 | C |
| ATOM | 873 | NZ | LYS | A | 55 | 6.708 | 44.412 | 6.017 | 0.00 | 20.00 | N |
| ATOM | 877 | C | LYS | A | 55 | 9.741 | 39.169 | 9.015 | 1.00 | 26.13 | C |
| ATOM | 878 | O | LYS | A | 55 | 9.486 | 38.240 | 8.254 | 1.00 | 25.62 | O |
| ATOM | 880 | N | VAL | A | 56 | 9.183 | 39.309 | 10.206 | 1.00 | 25.77 | N |
| ATOM | 881 | CA | VAL | A | 56 | 8.238 | 38.343 | 10.735 | 1.00 | 26.74 | C |
| ATOM | 883 | CB | VAL | A | 56 | 8.875 | 37.481 | 11.849 | 1.00 | 26.56 | C |
| ATOM | 885 | CG1 | VAL | A | 56 | 10.064 | 36.727 | 11.307 | 1.00 | 27.61 | C |
| ATOM | 889 | CG2 | VAL | A | 56 | 9.295 | 38.334 | 13.028 | 1.00 | 27.76 | C |
| ATOM | 893 | C | VAL | A | 56 | 7.036 | 39.082 | 11.288 | 1.00 | 27.17 | C |
| ATOM | 894 | O | VAL | A | 56 | 7.050 | 40.310 | 11.379 | 1.00 | 26.20 | O |
| ATOM | 896 | N | ARG | A | 57 | 5.992 | 38.327 | 11.621 | 1.00 | 27.13 | N |
| ATOM | 897 | CA | ARG | A | 57 | 4.823 | 38.864 | 12.305 | 1.00 | 27.81 | C |
| ATOM | 899 | CB | ARG | A | 57 | 3.536 | 38.333 | 11.672 | 1.00 | 27.24 | C |
| ATOM | 902 | CG | ARG | A | 57 | 3.400 | 38.566 | 10.175 | 1.00 | 29.02 | C |
| ATOM | 905 | CD | ARG | A | 57 | 2.021 | 38.108 | 9.668 | 1.00 | 31.21 | C |
| ATOM | 908 | NE | ARG | A | 57 | 2.082 | 36.821 | 8.972 | 1.00 | 32.83 | N |
| ATOM | 910 | CZ | ARG | A | 57 | 1.392 | 35.729 | 9.294 | 1.00 | 36.19 | C |
| ATOM | 911 | NH1 | ARG | A | 57 | 0.533 | 35.723 | 10.302 | 1.00 | 38.70 | N |
| ATOM | 914 | NH2 | ARG | A | 57 | 1.543 | 34.628 | 8.576 | 1.00 | 37.71 | N |
| ATOM | 917 | C | ARG | A | 57 | 4.886 | 38.393 | 13.749 | 1.00 | 27.15 | C |
| ATOM | 918 | O | ARG | A | 57 | 4.979 | 37.195 | 14.005 | 1.00 | 26.23 | O |
| ATOM | 920 | N | GLN | A | 58 | 4.842 | 39.323 | 14.694 | 1.00 | 26.43 | N |
| ATOM | 921 | CA | GLN | A | 58 | 4.886 | 38.954 | 16.102 | 1.00 | 26.89 | C |
| ATOM | 923 | CB | GLN | A | 58 | 5.675 | 39.996 | 16.904 | 1.00 | 27.67 | C |
| ATOM | 926 | CG | GLN | A | 58 | 5.837 | 39.658 | 18.380 | 1.00 | 28.57 | C |
| ATOM | 929 | CD | GLN | A | 58 | 6.396 | 40.808 | 19.191 | 1.00 | 28.77 | C |
| ATOM | 930 | OE1 | GLN | A | 58 | 7.265 | 41.549 | 18.733 | 1.00 | 33.38 | O |
| ATOM | 931 | NE2 | GLN | A | 58 | 5.897 | 40.964 | 20.410 | 1.00 | 31.28 | N |
| ATOM | 934 | C | GLN | A | 58 | 3.457 | 38.822 | 16.633 | 1.00 | 26.57 | C |
| ATOM | 935 | O | GLN | A | 58 | 2.664 | 39.759 | 16.525 | 1.00 | 26.01 | O |
| ATOM | 937 | N | TYR | A | 59 | 3.135 | 37.637 | 17.148 | 1.00 | 26.55 | N |
| ATOM | 938 | CA | TYR | A | 59 | 1.876 | 37.358 | 17.850 | 1.00 | 27.04 | C |
| ATOM | 940 | CB | TYR | A | 59 | 1.190 | 36.145 | 17.232 | 1.00 | 26.74 | C |
| ATOM | 943 | CG | TYR | A | 59 | 0.687 | 36.346 | 15.829 | 1.00 | 26.21 | C |
| ATOM | 944 | CD1 | TYR | A | 59 | 1.527 | 36.165 | 14.732 | 1.00 | 25.67 | C |
| ATOM | 946 | CE1 | TYR | A | 59 | 1.060 | 36.325 | 13.444 | 1.00 | 24.38 | C |
| ATOM | 948 | CZ | TYR | A | 59 | −0.277 | 36.629 | 13.243 | 1.00 | 26.85 | C |
| ATOM | 949 | OH | TYR | A | 59 | −0.786 | 36.805 | 11.973 | 1.00 | 26.63 | O |
| ATOM | 951 | CE2 | TYR | A | 59 | −1.124 | 36.805 | 14.317 | 1.00 | 26.32 | C |
| ATOM | 953 | CD2 | TYR | A | 59 | −0.643 | 36.649 | 15.595 | 1.00 | 25.70 | C |
| ATOM | 955 | C | TYR | A | 59 | 2.163 | 37.014 | 19.306 | 1.00 | 27.00 | C |
| ATOM | 956 | O | TYR | A | 59 | 3.046 | 36.215 | 19.588 | 1.00 | 28.03 | O |
| ATOM | 958 | N | ASP | A | 60 | 1.391 | 37.578 | 20.223 | 1.00 | 27.64 | N |
| ATOM | 959 | CA | ASP | A | 60 | 1.642 | 37.427 | 21.659 | 1.00 | 27.82 | C |
| ATOM | 961 | CB | ASP | A | 60 | 1.568 | 38.792 | 22.360 | 1.00 | 27.86 | C |
| ATOM | 964 | CG | ASP | A | 60 | 2.711 | 39.721 | 21.971 | 1.00 | 29.18 | C |
| ATOM | 965 | OD1 | ASP | A | 60 | 3.822 | 39.235 | 21.679 | 1.00 | 28.35 | O |
| ATOM | 966 | OD2 | ASP | A | 60 | 2.502 | 40.952 | 21.971 | 1.00 | 31.60 | O |
| ATOM | 967 | C | ASP | A | 60 | 0.637 | 36.461 | 22.292 | 1.00 | 27.24 | C |
| ATOM | 968 | O | ASP | A | 60 | −0.464 | 36.280 | 21.777 | 1.00 | 26.59 | O |
| ATOM | 970 | N | GLN | A | 61 | 1.050 | 35.831 | 23.391 | 1.00 | 27.01 | N |
| ATOM | 971 | CA | GLN | A | 61 | 0.223 | 34.903 | 24.172 | 1.00 | 27.35 | C |
| ATOM | 973 | CB | GLN | A | 61 | −0.837 | 35.660 | 24.979 | 1.00 | 27.86 | C |
| ATOM | 976 | CG | GLN | A | 61 | −0.276 | 36.787 | 25.854 | 1.00 | 29.97 | C |
| ATOM | 979 | CD | GLN | A | 61 | 0.279 | 36.286 | 27.176 | 1.00 | 32.84 | C |
| ATOM | 980 | OE1 | GLN | A | 61 | 1.196 | 35.455 | 27.209 | 1.00 | 32.58 | O |
| ATOM | 981 | NE2 | GLN | A | 61 | −0.283 | 36.791 | 28.281 | 1.00 | 32.11 | N |
| ATOM | 984 | C | GLN | A | 61 | −0.434 | 33.804 | 23.339 | 1.00 | 26.83 | C |
| ATOM | 985 | O | GLN | A | 61 | −1.635 | 33.559 | 23.463 | 1.00 | 26.10 | O |
| ATOM | 987 | N | ILE | A | 62 | 0.368 | 33.141 | 22.505 | 1.00 | 26.05 | N |
| ATOM | 988 | CA | ILE | A | 62 | −0.107 | 32.025 | 21.692 | 1.00 | 25.85 | C |
| ATOM | 990 | CB | ILE | A | 62 | 0.591 | 31.996 | 20.314 | 1.00 | 25.80 | C |
| ATOM | 992 | CG1 | ILE | A | 62 | 0.220 | 33.238 | 19.492 | 1.00 | 26.72 | C |
| ATOM | 995 | CD1 | ILE | A | 62 | −1.267 | 33.411 | 19.234 | 1.00 | 28.14 | C |
| ATOM | 999 | CG2 | ILE | A | 62 | 0.247 | 30.721 | 19.567 | 1.00 | 24.88 | C |
| ATOM | 1003 | C | ILE | A | 62 | 0.203 | 30.722 | 22.418 | 1.00 | 25.13 | C |
| ATOM | 1004 | O | ILE | A | 62 | 1.337 | 30.516 | 22.820 | 1.00 | 25.00 | O |
| ATOM | 1006 | N | PRO | A | 63 | −0.807 | 29.861 | 22.623 | 1.00 | 25.00 | N |
| ATOM | 1007 | CA | PRO | A | 63 | −0.547 | 28.559 | 23.234 | 1.00 | 25.18 | C |
| ATOM | 1009 | CB | PRO | A | 63 | −1.943 | 28.081 | 23.673 | 1.00 | 25.10 | C |
| ATOM | 1012 | CG | PRO | A | 63 | −2.880 | 29.206 | 23.411 | 1.00 | 25.58 | C |
| ATOM | 1015 | CD | PRO | A | 63 | −2.239 | 30.054 | 22.359 | 1.00 | 25.21 | C |
| ATOM | 1018 | C | PRO | A | 63 | 0.059 | 27.580 | 22.235 | 1.00 | 24.80 | C |
| ATOM | 1019 | O | PRO | A | 63 | −0.474 | 27.409 | 21.140 | 1.00 | 24.01 | O |
| ATOM | 1020 | N | ILE | A | 64 | 1.182 | 26.979 | 22.612 | 1.00 | 25.40 | N |
| ATOM | 1021 | CA | ILE | A | 64 | 1.813 | 25.904 | 21.855 | 1.00 | 25.46 | C |
| ATOM | 1023 | CB | ILE | A | 64 | 3.169 | 26.344 | 21.239 | 1.00 | 25.54 | C |
| ATOM | 1025 | CG1 | ILE | A | 64 | 2.985 | 27.569 | 20.328 | 1.00 | 27.99 | C |
| ATOM | 1028 | CD1 | ILE | A | 64 | 4.192 | 27.875 | 19.424 | 1.00 | 27.80 | C |
| ATOM | 1032 | CG2 | ILE | A | 64 | 3.804 | 25.179 | 20.464 | 1.00 | 24.59 | C |
| ATOM | 1036 | C | ILE | A | 64 | 2.108 | 24.743 | 22.802 | 1.00 | 24.91 | C |
| ATOM | 1037 | O | ILE | A | 64 | 2.745 | 24.933 | 23.839 | 1.00 | 24.79 | O |
| ATOM | 1039 | N | GLU | A | 65 | 1.663 | 23.544 | 22.441 | 1.00 | 24.29 | N |
| ATOM | 1040 | CA | GLU | A | 65 | 2.048 | 22.342 | 23.173 | 1.00 | 25.25 | C |
| ATOM | 1042 | CB | GLU | A | 65 | 0.966 | 21.273 | 23.051 | 1.00 | 25.15 | C |
| ATOM | 1045 | CG | GLU | A | 65 | 1.177 | 20.068 | 23.949 | 1.00 | 27.40 | C |
| ATOM | 1048 | CD | GLU | A | 65 | −0.055 | 19.185 | 24.012 | 1.00 | 27.76 | C |
| ATOM | 1049 | OE1 | GLU | A | 65 | −0.806 | 19.155 | 23.017 | 1.00 | 32.12 | O |
| ATOM | 1050 | OE2 | GLU | A | 65 | −0.287 | 18.541 | 25.055 | 1.00 | 30.59 | O |
| ATOM | 1051 | C | GLU | A | 65 | 3.348 | 21.809 | 22.585 | 1.00 | 24.73 | C |
| ATOM | 1052 | O | GLU | A | 65 | 3.434 | 21.603 | 21.374 | 1.00 | 23.52 | O |
| ATOM | 1054 | N | ILE | A | 66 | 4.350 | 21.606 | 23.438 | 1.00 | 24.51 | N |
| ATOM | 1055 | CA | ILE | A | 66 | 5.634 | 21.052 | 23.009 | 1.00 | 24.92 | C |
| ATOM | 1057 | CB | ILE | A | 66 | 6.770 | 22.076 | 23.188 | 1.00 | 24.39 | C |
| ATOM | 1059 | CG1 | ILE | A | 66 | 6.451 | 23.384 | 22.461 | 1.00 | 25.44 | C |
| ATOM | 1062 | CD1 | ILE | A | 66 | 7.228 | 24.572 | 22.993 | 1.00 | 25.75 | C |
| ATOM | 1066 | CG2 | ILE | A | 66 | 8.094 | 21.499 | 22.699 | 1.00 | 24.32 | C |
| ATOM | 1070 | C | ILE | A | 66 | 5.948 | 19.801 | 23.830 | 1.00 | 25.42 | C |
| ATOM | 1071 | O | ILE | A | 66 | 6.167 | 19.884 | 25.032 | 1.00 | 23.99 | O |
| ATOM | 1073 | N | CYS | A | 67 | 5.964 | 18.644 | 23.173 | 1.00 | 27.06 | N |
| ATOM | 1074 | CA | CYS | A | 67 | 6.120 | 17.350 | 23.850 | 1.00 | 28.17 | C |
| ATOM | 1076 | CB | CYS | A | 67 | 7.557 | 17.151 | 24.308 | 1.00 | 28.60 | C |
| ATOM | 1079 | SG | CYS | A | 67 | 8.689 | 17.069 | 22.962 | 1.00 | 32.25 | S |
| ATOM | 1081 | C | CYS | A | 67 | 5.232 | 17.218 | 25.063 | 1.00 | 28.50 | C |
| ATOM | 1082 | O | CYS | A | 67 | 5.706 | 16.848 | 26.139 | 1.00 | 28.67 | O |
| ATOM | 1084 | N | GLY | A | 68 | 3.962 | 17.569 | 24.910 | 1.00 | 28.74 | N |
| ATOM | 1085 | CA | GLY | A | 68 | 3.019 | 17.467 | 26.011 | 1.00 | 29.11 | C |
| ATOM | 1088 | C | GLY | A | 68 | 3.089 | 18.607 | 27.011 | 1.00 | 28.88 | C |
| ATOM | 1089 | O | GLY | A | 68 | 2.225 | 18.705 | 27.881 | 1.00 | 29.65 | O |
| ATOM | 1091 | N | HIS | A | 69 | 4.092 | 19.476 | 26.898 | 1.00 | 27.92 | N |
| ATOM | 1092 | CA | HIS | A | 69 | 4.216 | 20.606 | 27.816 | 1.00 | 27.07 | C |
| ATOM | 1094 | CB | HIS | A | 69 | 5.678 | 20.983 | 28.038 | 1.00 | 26.44 | C |
| ATOM | 1097 | CG | HIS | A | 69 | 6.457 | 19.974 | 28.814 | 1.00 | 25.81 | C |
| ATOM | 1098 | ND1 | HIS | A | 69 | 6.754 | 18.723 | 28.320 | 1.00 | 24.59 | N |
| ATOM | 1100 | CE1 | HIS | A | 69 | 7.475 | 18.065 | 29.210 | 1.00 | 26.05 | C |
| ATOM | 1102 | NE2 | HIS | A | 69 | 7.660 | 18.844 | 30.260 | 1.00 | 23.46 | N |
| ATOM | 1104 | CD2 | HIS | A | 69 | 7.035 | 20.045 | 30.037 | 1.00 | 24.42 | C |
| ATOM | 1106 | C | HIS | A | 69 | 3.511 | 21.809 | 27.227 | 1.00 | 27.37 | C |
| ATOM | 1107 | O | HIS | A | 69 | 3.776 | 22.168 | 26.083 | 1.00 | 25.98 | O |
| ATOM | 1109 | N | LYS | A | 70 | 2.670 | 22.456 | 28.035 | 1.00 | 27.44 | N |
| ATOM | 1110 | CA | LYS | A | 70 | 1.949 | 23.661 | 27.630 | 1.00 | 27.66 | C |
| ATOM | 1112 | CB | LYS | A | 70 | 0.665 | 23.830 | 28.454 | 1.00 | 27.82 | C |
| ATOM | 1119 | C | LYS | A | 70 | 2.814 | 24.909 | 27.771 | 1.00 | 28.05 | C |
| ATOM | 1120 | O | LYS | A | 70 | 3.383 | 25.185 | 28.834 | 1.00 | 28.02 | O |
| ATOM | 1122 | N | ALA | A | 71 | 2.953 | 25.628 | 26.665 | 1.00 | 27.91 | N |
| ATOM | 1123 | CA | ALA | A | 71 | 3.644 | 26.899 | 26.645 | 1.00 | 27.47 | C |
| ATOM | 1125 | CB | ALA | A | 71 | 4.861 | 26.831 | 25.742 | 1.00 | 28.14 | C |
| ATOM | 1129 | C | ALA | A | 71 | 2.660 | 27.920 | 26.113 | 1.00 | 27.19 | C |
| ATOM | 1130 | O | ALA | A | 71 | 1.744 | 27.577 | 25.371 | 1.00 | 27.00 | O |
| ATOM | 1132 | N | ILE | A | 72 | 2.837 | 29.171 | 26.519 | 1.00 | 26.83 | N |
| ATOM | 1133 | CA | ILE | A | 72 | 2.036 | 30.268 | 25.991 | 1.00 | 26.48 | C |
| ATOM | 1135 | CB | ILE | A | 72 | 0.841 | 30.627 | 26.883 | 1.00 | 26.03 | C |
| ATOM | 1137 | CG1 | ILE | A | 72 | 0.007 | 29.397 | 27.188 | 1.00 | 26.93 | C |
| ATOM | 1140 | CD1 | ILE | A | 72 | −1.248 | 29.736 | 27.948 | 1.00 | 27.09 | C |
| ATOM | 1144 | CG2 | ILE | A | 72 | −0.041 | 31.686 | 26.207 | 1.00 | 26.13 | C |
| ATOM | 1148 | C | ILE | A | 72 | 2.929 | 31.478 | 25.941 | 1.00 | 25.75 | C |
| ATOM | 1149 | O | ILE | A | 72 | 3.483 | 31.885 | 26.953 | 1.00 | 25.08 | O |
| ATOM | 1151 | N | GLY | A | 73 | 3.078 | 32.053 | 24.765 | 1.00 | 25.73 | N |
| ATOM | 1152 | CA | GLY | A | 73 | 3.928 | 33.211 | 24.659 | 1.00 | 26.08 | C |
| ATOM | 1155 | C | GLY | A | 73 | 4.003 | 33.755 | 23.261 | 1.00 | 26.25 | C |
| ATOM | 1156 | O | GLY | A | 73 | 3.118 | 33.540 | 22.430 | 1.00 | 24.72 | O |
| ATOM | 1158 | N | THR | A | 74 | 5.098 | 34.456 | 23.012 | 1.00 | 26.37 | N |
| ATOM | 1159 | CA | THR | A | 74 | 5.272 | 35.157 | 21.766 | 1.00 | 26.92 | C |
| ATOM | 1161 | CB | THR | A | 74 | 6.230 | 36.342 | 21.934 | 1.00 | 26.45 | C |
| ATOM | 1163 | OG1 | THR | A | 74 | 5.546 | 37.361 | 22.664 | 1.00 | 26.38 | O |
| ATOM | 1165 | CG2 | THR | A | 74 | 6.660 | 36.891 | 20.578 | 1.00 | 27.86 | C |
| ATOM | 1169 | C | THR | A | 74 | 5.748 | 34.209 | 20.689 | 1.00 | 27.40 | C |
| ATOM | 1170 | O | THR | A | 74 | 6.617 | 33.370 | 20.904 | 1.00 | 27.93 | O |
| ATOM | 1172 | N | VAL | A | 75 | 5.120 | 34.333 | 19.532 | 1.00 | 27.96 | N |
| ATOM | 1173 | CA | VAL | A | 75 | 5.451 | 33.520 | 18.382 | 1.00 | 28.35 | C |
| ATOM | 1175 | CB | VAL | A | 75 | 4.302 | 32.525 | 18.088 | 1.00 | 28.64 | C |
| ATOM | 1177 | CG1 | VAL | A | 75 | 4.554 | 31.779 | 16.801 | 1.00 | 30.34 | C |
| ATOM | 1181 | CG2 | VAL | A | 75 | 4.135 | 31.559 | 19.264 | 1.00 | 28.49 | C |
| ATOM | 1185 | C | VAL | A | 75 | 5.651 | 34.487 | 17.222 | 1.00 | 28.10 | C |
| ATOM | 1186 | O | VAL | A | 75 | 4.833 | 35.388 | 17.000 | 1.00 | 26.65 | O |
| ATOM | 1188 | N | LEU | A | 76 | 6.767 | 34.321 | 16.519 | 1.00 | 28.53 | N |
| ATOM | 1189 | CA | LEU | A | 76 | 7.058 | 35.115 | 15.334 | 1.00 | 28.18 | C |
| ATOM | 1191 | CB | LEU | A | 76 | 8.511 | 35.580 | 15.363 | 1.00 | 28.39 | C |
| ATOM | 1194 | CG | LEU | A | 76 | 9.039 | 36.215 | 16.651 | 1.00 | 28.30 | C |
| ATOM | 1196 | CD1 | LEU | A | 76 | 10.467 | 36.648 | 16.425 | 1.00 | 29.01 | C |
| ATOM | 1200 | CD2 | LEU | A | 76 | 8.207 | 37.429 | 17.053 | 1.00 | 27.79 | C |
| ATOM | 1204 | C | LEU | A | 76 | 6.805 | 34.262 | 14.095 | 1.00 | 28.22 | C |
| ATOM | 1205 | O | LEU | A | 76 | 7.292 | 33.134 | 14.000 | 1.00 | 29.02 | O |
| ATOM | 1207 | N | VAL | A | 77 | 6.029 | 34.775 | 13.152 | 1.00 | 26.77 | N |
| ATOM | 1208 | CA | VAL | A | 77 | 5.709 | 33.999 | 11.953 | 1.00 | 26.07 | C |
| ATOM | 1210 | CB | VAL | A | 77 | 4.188 | 33.857 | 11.753 | 1.00 | 25.35 | C |
| ATOM | 1212 | CG1 | VAL | A | 77 | 3.881 | 33.055 | 10.490 | 1.00 | 24.94 | C |
| ATOM | 1216 | CG2 | VAL | A | 77 | 3.561 | 33.186 | 12.972 | 1.00 | 24.61 | C |
| ATOM | 1220 | C | VAL | A | 77 | 6.351 | 34.635 | 10.728 | 1.00 | 25.35 | C |
| ATOM | 1221 | O | VAL | A | 77 | 6.180 | 35.820 | 10.457 | 1.00 | 25.13 | O |
| ATOM | 1223 | N | GLY | A | 78 | 7.090 | 33.837 | 9.976 | 1.00 | 25.57 | N |
| ATOM | 1224 | CA | GLY | A | 78 | 7.768 | 34.365 | 8.812 | 1.00 | 25.78 | C |
| ATOM | 1227 | C | GLY | A | 78 | 8.346 | 33.267 | 7.968 | 1.00 | 25.97 | C |
| ATOM | 1228 | O | GLY | A | 78 | 8.149 | 32.092 | 8.273 | 1.00 | 26.99 | O |
| ATOM | 1230 | N | PRO | A | 79 | 9.093 | 33.649 | 6.924 | 1.00 | 26.57 | N |
| ATOM | 1231 | CA | PRO | A | 79 | 9.644 | 32.719 | 5.945 | 1.00 | 27.52 | C |
| ATOM | 1233 | CB | PRO | A | 79 | 10.018 | 33.620 | 4.760 | 1.00 | 27.24 | C |
| ATOM | 1236 | CG | PRO | A | 79 | 10.247 | 34.961 | 5.352 | 1.00 | 28.40 | C |
| ATOM | 1239 | CD | PRO | A | 79 | 9.418 | 35.052 | 6.611 | 1.00 | 27.56 | C |
| ATOM | 1242 | C | PRO | A | 79 | 10.838 | 31.930 | 6.456 | 1.00 | 27.82 | C |
| ATOM | 1243 | O | PRO | A | 79 | 11.985 | 32.262 | 6.156 | 1.00 | 27.46 | O |
| ATOM | 1244 | N | THR | A | 80 | 10.537 | 30.899 | 7.242 | 1.00 | 28.78 | N |
| ATOM | 1245 | CA | THR | A | 80 | 11.523 | 29.932 | 7.710 | 1.00 | 28.56 | C |
| ATOM | 1247 | CB | THR | A | 80 | 11.598 | 29.932 | 9.275 | 1.00 | 29.49 | C |
| ATOM | 1249 | OG1 | THR | A | 80 | 12.579 | 28.992 | 9.741 | 1.00 | 30.55 | O |
| ATOM | 1251 | CG2 | THR | A | 80 | 10.244 | 29.613 | 9.920 | 1.00 | 27.76 | C |
| ATOM | 1255 | C | THR | A | 80 | 11.151 | 28.552 | 7.141 | 1.00 | 28.82 | C |
| ATOM | 1256 | O | THR | A | 80 | 9.971 | 28.190 | 7.090 | 1.00 | 28.30 | O |
| ATOM | 1258 | N | PRO | A | 81 | 12.155 | 27.774 | 6.706 | 1.00 | 28.88 | N |
| ATOM | 1259 | CA | PRO | A | 81 | 11.920 | 26.377 | 6.320 | 1.00 | 29.33 | C |
| ATOM | 1261 | CB | PRO | A | 81 | 13.228 | 25.986 | 5.633 | 1.00 | 29.40 | C |
| ATOM | 1264 | CG | PRO | A | 81 | 14.257 | 26.818 | 6.326 | 1.00 | 29.42 | C |
| ATOM | 1267 | CD | PRO | A | 81 | 13.585 | 28.130 | 6.629 | 1.00 | 28.73 | C |
| ATOM | 1270 | C | PRO | A | 81 | 11.697 | 25.457 | 7.518 | 1.00 | 29.29 | C |
| ATOM | 1271 | O | PRO | A | 81 | 11.170 | 24.351 | 7.358 | 1.00 | 29.63 | O |
| ATOM | 1272 | N | VAL | A | 82 | 12.122 | 25.906 | 8.699 | 1.00 | 28.71 | N |
| ATOM | 1273 | CA | VAL | A | 82 | 12.036 | 25.105 | 9.914 | 1.00 | 28.30 | C |
| ATOM | 1275 | CB | VAL | A | 82 | 13.441 | 24.572 | 10.333 | 1.00 | 28.84 | C |
| ATOM | 1277 | CG1 | VAL | A | 82 | 14.487 | 25.674 | 10.323 | 1.00 | 29.68 | C |
| ATOM | 1281 | CG2 | VAL | A | 82 | 13.396 | 23.901 | 11.695 | 1.00 | 29.19 | C |
| ATOM | 1285 | C | VAL | A | 82 | 11.399 | 25.909 | 11.037 | 1.00 | 27.24 | C |
| ATOM | 1286 | O | VAL | A | 82 | 11.796 | 27.038 | 11.304 | 1.00 | 27.29 | O |
| ATOM | 1288 | N | ASN | A | 83 | 10.399 | 25.333 | 11.696 | 1.00 | 26.83 | N |
| ATOM | 1289 | CA | ASN | A | 83 | 9.905 | 25.892 | 12.961 | 1.00 | 26.53 | C |
| ATOM | 1291 | CB | ASN | A | 83 | 8.689 | 25.105 | 13.468 | 1.00 | 26.68 | C |
| ATOM | 1294 | CG | ASN | A | 83 | 7.508 | 25.178 | 12.521 | 1.00 | 29.23 | C |
| ATOM | 1295 | OD1 | ASN | A | 83 | 7.072 | 26.266 | 12.157 | 1.00 | 28.74 | O |
| ATOM | 1296 | ND2 | ASN | A | 83 | 6.992 | 24.015 | 12.112 | 1.00 | 27.10 | N |
| ATOM | 1299 | C | ASN | A | 83 | 11.023 | 25.812 | 14.000 | 1.00 | 26.25 | C |
| ATOM | 1300 | O | ASN | A | 83 | 11.645 | 24.761 | 14.177 | 1.00 | 24.17 | O |
| ATOM | 1302 | N | ILE | A | 84 | 11.301 | 26.925 | 14.667 | 1.00 | 26.49 | N |
| ATOM | 1303 | CA | ILE | A | 84 | 12.303 | 26.930 | 15.708 | 1.00 | 27.40 | C |
| ATOM | 1305 | CB | ILE | A | 84 | 13.531 | 27.822 | 15.361 | 1.00 | 27.73 | C |
| ATOM | 1307 | CG1 | ILE | A | 84 | 13.218 | 29.309 | 15.469 | 1.00 | 29.59 | C |
| ATOM | 1310 | CD1 | ILE | A | 84 | 14.351 | 30.190 | 14.951 | 1.00 | 31.11 | C |
| ATOM | 1314 | CG2 | ILE | A | 84 | 14.039 | 27.512 | 13.955 | 1.00 | 28.56 | C |
| ATOM | 1318 | C | ILE | A | 84 | 11.661 | 27.332 | 17.017 | 1.00 | 26.92 | C |
| ATOM | 1319 | O | ILE | A | 84 | 10.952 | 28.332 | 17.091 | 1.00 | 27.41 | O |
| ATOM | 1321 | N | ILE | A | 85 | 11.912 | 26.534 | 18.046 | 1.00 | 26.99 | N |
| ATOM | 1322 | CA | ILE | A | 85 | 11.460 | 26.851 | 19.388 | 1.00 | 27.19 | C |
| ATOM | 1324 | CB | ILE | A | 85 | 10.977 | 25.600 | 20.149 | 1.00 | 27.40 | C |
| ATOM | 1326 | CG1 | ILE | A | 85 | 9.912 | 24.844 | 19.347 | 1.00 | 26.27 | C |
| ATOM | 1329 | CD1 | ILE | A | 85 | 8.678 | 25.649 | 18.957 | 1.00 | 26.46 | C |
| ATOM | 1333 | CG2 | ILE | A | 85 | 10.441 | 25.989 | 21.529 | 1.00 | 28.18 | C |
| ATOM | 1337 | C | ILE | A | 85 | 12.603 | 27.523 | 20.137 | 1.00 | 27.09 | C |
| ATOM | 1338 | O | ILE | A | 85 | 13.642 | 26.910 | 20.401 | 1.00 | 27.38 | O |
| ATOM | 1340 | N | GLY | A | 86 | 12.409 | 28.804 | 20.442 | 1.00 | 26.58 | N |
| ATOM | 1341 | CA | GLY | A | 86 | 13.423 | 29.598 | 21.106 | 1.00 | 26.03 | C |
| ATOM | 1344 | C | GLY | A | 86 | 13.319 | 29.653 | 22.615 | 1.00 | 25.81 | C |
| ATOM | 1345 | O | GLY | A | 86 | 12.433 | 29.050 | 23.216 | 1.00 | 27.11 | O |
| ATOM | 1347 | N | ARG | A | 87 | 14.232 | 30.408 | 23.225 | 1.00 | 25.46 | N |
| ATOM | 1348 | CA | ARG | A | 87 | 14.370 | 30.458 | 24.680 | 1.00 | 26.32 | C |
| ATOM | 1350 | CB | ARG | A | 87 | 15.543 | 31.357 | 25.065 | 1.00 | 25.41 | C |
| ATOM | 1353 | CG | ARG | A | 87 | 16.890 | 30.726 | 24.753 | 1.00 | 26.18 | C |
| ATOM | 1356 | CD | ARG | A | 87 | 18.063 | 31.545 | 25.309 | 1.00 | 25.42 | C |
| ATOM | 1359 | NE | ARG | A | 87 | 18.046 | 32.946 | 24.891 | 1.00 | 25.40 | N |
| ATOM | 1361 | CZ | ARG | A | 87 | 17.584 | 33.967 | 25.612 | 1.00 | 24.82 | C |
| ATOM | 1362 | NH1 | ARG | A | 87 | 17.102 | 33.796 | 26.838 | 1.00 | 23.66 | N |
| ATOM | 1365 | NH2 | ARG | A | 87 | 17.627 | 35.184 | 25.104 | 1.00 | 23.61 | N |
| ATOM | 1368 | C | ARG | A | 87 | 13.092 | 30.915 | 25.384 | 1.00 | 26.30 | C |
| ATOM | 1369 | O | ARG | A | 87 | 12.765 | 30.425 | 26.461 | 1.00 | 26.53 | O |
| ATOM | 1371 | N | ASN | A | 88 | 12.334 | 31.791 | 24.729 | 1.00 | 26.22 | N |
| ATOM | 1372 | CA | ASN | A | 88 | 11.137 | 32.339 | 25.334 | 1.00 | 26.85 | C |
| ATOM | 1374 | CB | ASN | A | 88 | 10.501 | 33.430 | 24.453 | 1.00 | 27.03 | C |
| ATOM | 1377 | CG | ASN | A | 88 | 9.765 | 32.861 | 23.248 | 1.00 | 25.75 | C |
| ATOM | 1378 | OD1 | ASN | A | 88 | 10.369 | 32.223 | 22.398 | 1.00 | 25.63 | O |
| ATOM | 1379 | ND2 | ASN | A | 88 | 8.453 | 33.098 | 23.176 | 1.00 | 23.93 | N |
| ATOM | 1382 | C | ASN | A | 88 | 10.146 | 31.230 | 25.670 | 1.00 | 26.73 | C |
| ATOM | 1383 | O | ASN | A | 88 | 9.432 | 31.333 | 26.661 | 1.00 | 27.22 | O |
| ATOM | 1385 | N | LEU | A | 89 | 10.138 | 30.151 | 24.886 | 1.00 | 27.10 | N |
| ATOM | 1386 | CA | LEU | A | 89 | 9.281 | 28.999 | 25.189 | 1.00 | 26.64 | C |
| ATOM | 1388 | CB | LEU | A | 89 | 8.527 | 28.525 | 23.946 | 1.00 | 27.02 | C |
| ATOM | 1391 | CG | LEU | A | 89 | 7.578 | 29.542 | 23.300 | 1.00 | 26.14 | C |
| ATOM | 1393 | CD1 | LEU | A | 89 | 6.713 | 28.868 | 22.257 | 1.00 | 25.83 | C |
| ATOM | 1397 | CD2 | LEU | A | 89 | 6.713 | 30.231 | 24.342 | 1.00 | 28.43 | C |
| ATOM | 1401 | C | LEU | A | 89 | 10.037 | 27.834 | 25.818 | 1.00 | 26.60 | C |
| ATOM | 1402 | O | LEU | A | 89 | 9.460 | 27.065 | 26.595 | 1.00 | 26.73 | O |
| ATOM | 1404 | N | LEU | A | 90 | 11.329 | 27.728 | 25.524 | 1.00 | 26.23 | N |
| ATOM | 1405 | CA | LEU | A | 90 | 12.139 | 26.640 | 26.063 | 1.00 | 26.84 | C |
| ATOM | 1407 | CB | LEU | A | 90 | 13.545 | 26.645 | 25.449 | 1.00 | 26.73 | C |
| ATOM | 1410 | CG | LEU | A | 90 | 13.689 | 26.264 | 23.979 | 1.00 | 26.99 | C |
| ATOM | 1412 | CD1 | LEU | A | 90 | 15.165 | 26.358 | 23.587 | 1.00 | 26.16 | C |
| ATOM | 1416 | CD2 | LEU | A | 90 | 13.150 | 24.872 | 23.708 | 1.00 | 27.43 | C |
| ATOM | 1420 | C | LEU | A | 90 | 12.221 | 26.749 | 27.582 | 1.00 | 26.58 | C |
| ATOM | 1421 | O | LEU | A | 90 | 12.083 | 25.760 | 28.294 | 1.00 | 26.73 | O |
| ATOM | 1423 | N | THR | A | 91 | 12.358 | 27.977 | 28.073 | 1.00 | 27.03 | N |
| ATOM | 1424 | CA | THR | A | 91 | 12.334 | 28.241 | 29.509 | 1.00 | 26.13 | C |
| ATOM | 1426 | CB | THR | A | 91 | 12.544 | 29.731 | 29.789 | 1.00 | 26.65 | C |
| ATOM | 1428 | OG1 | THR | A | 91 | 11.619 | 30.518 | 29.015 | 1.00 | 25.97 | O |
| ATOM | 1430 | CG2 | THR | A | 91 | 13.958 | 30.135 | 29.465 | 1.00 | 27.26 | C |
| ATOM | 1434 | C | THR | A | 91 | 11.021 | 27.814 | 30.162 | 1.00 | 26.18 | C |
| ATOM | 1435 | O | THR | A | 91 | 11.024 | 27.200 | 31.230 | 1.00 | 26.16 | O |
| ATOM | 1437 | N | GLN | A | 92 | 9.899 | 28.108 | 29.512 | 1.00 | 25.60 | N |
| ATOM | 1438 | CA | GLN | A | 92 | 8.579 | 27.774 | 30.067 | 1.00 | 25.37 | C |
| ATOM | 1440 | CB | GLN | A | 92 | 7.466 | 28.292 | 29.158 | 1.00 | 25.12 | C |
| ATOM | 1443 | CG | GLN | A | 92 | 7.400 | 29.799 | 28.972 | 1.00 | 24.67 | C |
| ATOM | 1446 | CD | GLN | A | 92 | 6.163 | 30.211 | 28.183 | 1.00 | 26.73 | C |
| ATOM | 1447 | OE1 | GLN | A | 92 | 5.248 | 29.408 | 27.976 | 1.00 | 27.25 | O |
| ATOM | 1448 | NE2 | GLN | A | 92 | 6.124 | 31.466 | 27.745 | 1.00 | 31.93 | N |
| ATOM | 1451 | C | GLN | A | 92 | 8.369 | 26.271 | 30.279 | 1.00 | 24.93 | C |
| ATOM | 1452 | O | GLN | A | 92 | 7.647 | 25.856 | 31.188 | 1.00 | 24.42 | O |
| ATOM | 1454 | N | ILE | A | 93 | 8.972 | 25.444 | 29.431 | 1.00 | 25.12 | N |
| ATOM | 1455 | CA | ILE | A | 93 | 8.826 | 23.999 | 29.589 | 1.00 | 25.31 | C |
| ATOM | 1457 | CB | ILE | A | 93 | 8.689 | 23.298 | 28.221 | 1.00 | 25.20 | C |
| ATOM | 1459 | CG1 | ILE | A | 93 | 10.011 | 23.335 | 27.449 | 1.00 | 25.27 | C |
| ATOM | 1462 | CD1 | ILE | A | 93 | 9.972 | 22.597 | 26.154 | 1.00 | 25.37 | C |
| ATOM | 1466 | CG2 | ILE | A | 93 | 7.573 | 23.971 | 27.395 | 1.00 | 25.44 | C |
| ATOM | 1470 | C | ILE | A | 93 | 9.967 | 23.404 | 30.428 | 1.00 | 26.30 | C |
| ATOM | 1471 | O | ILE | A | 93 | 10.051 | 22.186 | 30.614 | 1.00 | 25.59 | O |
| ATOM | 1473 | N | GLY | A | 94 | 10.855 | 24.268 | 30.918 | 1.00 | 26.83 | N |
| ATOM | 1474 | CA | GLY | A | 94 | 11.863 | 23.859 | 31.894 | 1.00 | 27.12 | C |
| ATOM | 1477 | C | GLY | A | 94 | 13.056 | 23.190 | 31.247 | 1.00 | 27.58 | C |
| ATOM | 1478 | O | GLY | A | 94 | 13.698 | 22.327 | 31.852 | 1.00 | 26.40 | O |
| ATOM | 1480 | N | CYS | A | 95 | 13.352 | 23.605 | 30.019 | 1.00 | 28.86 | N |
| ATOM | 1481 | CA | CYS | A | 95 | 14.430 | 23.017 | 29.230 | 1.00 | 28.67 | C |
| ATOM | 1483 | CB | CYS | A | 95 | 14.164 | 23.259 | 27.741 | 1.00 | 28.56 | C |
| ATOM | 1486 | SG | CYS | A | 95 | 15.282 | 22.341 | 26.705 | 1.00 | 32.93 | S |
| ATOM | 1488 | C | CYS | A | 95 | 15.797 | 23.576 | 29.626 | 1.00 | 28.29 | C |
| ATOM | 1489 | O | CYS | A | 95 | 15.989 | 24.792 | 29.676 | 1.00 | 29.45 | O |
| ATOM | 1491 | N | THR | A | 96 | 16.737 | 22.695 | 29.953 | 1.00 | 28.13 | N |
| ATOM | 1492 | CA | THR | A | 96 | 18.109 | 23.106 | 30.241 | 1.00 | 27.12 | C |
| ATOM | 1494 | CB | THR | A | 96 | 18.517 | 22.807 | 31.708 | 1.00 | 27.09 | C |
| ATOM | 1496 | OG1 | THR | A | 96 | 18.519 | 21.395 | 31.930 | 1.00 | 25.69 | O |
| ATOM | 1498 | CG2 | THR | A | 96 | 17.563 | 23.469 | 32.699 | 1.00 | 27.33 | C |
| ATOM | 1502 | C | THR | A | 96 | 19.109 | 22.426 | 29.309 | 1.00 | 26.82 | C |
| ATOM | 1503 | O | THR | A | 96 | 18.806 | 21.439 | 28.629 | 1.00 | 26.03 | O |
| ATOM | 1505 | N | LEU | A | 97 | 20.301 | 23.000 | 29.252 | 1.00 | 26.22 | N |
| ATOM | 1506 | CA | LEU | A | 97 | 21.418 | 22.369 | 28.582 | 1.00 | 25.83 | C |
| ATOM | 1508 | CB | LEU | A | 97 | 22.240 | 23.402 | 27.817 | 1.00 | 26.51 | C |
| ATOM | 1511 | CG | LEU | A | 97 | 21.735 | 23.854 | 26.450 | 1.00 | 25.78 | C |
| ATOM | 1513 | CD1 | LEU | A | 97 | 22.423 | 25.156 | 26.091 | 1.00 | 25.40 | C |
| ATOM | 1517 | CD2 | LEU | A | 97 | 22.018 | 22.768 | 25.423 | 1.00 | 24.50 | C |
| ATOM | 1521 | C | LEU | A | 97 | 22.277 | 21.766 | 29.670 | 1.00 | 25.84 | C |
| ATOM | 1522 | O | LEU | A | 97 | 22.474 | 22.379 | 30.711 | 1.00 | 25.45 | O |
| ATOM | 1524 | N | ASN | A | 98 | 22.800 | 20.574 | 29.434 | 1.00 | 26.55 | N |
| ATOM | 1525 | CA | ASN | A | 98 | 23.550 | 19.864 | 30.470 | 1.00 | 27.32 | C |
| ATOM | 1527 | CB | ASN | A | 98 | 22.648 | 18.832 | 31.159 | 1.00 | 27.41 | C |
| ATOM | 1530 | CG | ASN | A | 98 | 21.418 | 19.467 | 31.818 | 1.00 | 29.43 | C |
| ATOM | 1531 | OD1 | ASN | A | 98 | 20.445 | 19.826 | 31.149 | 1.00 | 30.99 | O |
| ATOM | 1532 | ND2 | ASN | A | 98 | 21.455 | 19.588 | 33.133 | 1.00 | 29.73 | N |
| ATOM | 1535 | C | ASN | A | 98 | 24.797 | 19.193 | 29.903 | 1.00 | 27.70 | C |
| ATOM | 1536 | O | ASN | A | 98 | 24.737 | 18.498 | 28.876 | 1.00 | 27.37 | O |
| ATOM | 1538 | N | PHE | A | 99 | 25.933 | 19.456 | 30.541 | 1.00 | 28.11 | N |
| ATOM | 1539 | CA | PHE | A | 99 | 27.172 | 18.768 | 30.220 | 1.00 | 28.37 | C |
| ATOM | 1541 | CB | PHE | A | 99 | 27.790 | 19.294 | 28.915 | 1.00 | 29.19 | C |
| ATOM | 1544 | CG | PHE | A | 99 | 28.294 | 20.708 | 28.987 | 1.00 | 29.45 | C |
| ATOM | 1545 | CD1 | PHE | A | 99 | 27.433 | 21.777 | 28.779 | 1.00 | 30.19 | C |
| ATOM | 1547 | CE1 | PHE | A | 99 | 27.892 | 23.088 | 28.824 | 1.00 | 30.24 | C |
| ATOM | 1549 | CZ | PHE | A | 99 | 29.230 | 23.339 | 29.043 | 1.00 | 29.69 | C |
| ATOM | 1551 | CE2 | PHE | A | 99 | 30.109 | 22.280 | 29.226 | 1.00 | 30.85 | C |
| ATOM | 1553 | CD2 | PHE | A | 99 | 29.642 | 20.970 | 29.186 | 1.00 | 29.90 | C |
| ATOM | 1555 | C | PHE | A | 99 | 28.175 | 18.845 | 31.364 | 1.00 | 28.49 | C |
| ATOM | 1556 | O | PHE | A | 99 | 29.257 | 18.241 | 31.290 | 1.00 | 27.34 | O |
| ATOM | 1558 | OXT | PHE | A | 99 | 27.892 | 19.475 | 32.394 | 1.00 | 28.60 | O |
| TER | | | | | | | | | | | |
| ATOM | 1559 | N | PRO | B | 1 | 28.310 | 21.708 | 33.846 | 1.00 | 28.89 | N |
| ATOM | 1560 | CA | PRO | B | 1 | 27.277 | 22.611 | 34.359 | 1.00 | 28.67 | C |
| ATOM | 1562 | CB | PRO | B | 1 | 27.735 | 23.983 | 33.859 | 1.00 | 28.59 | C |
| ATOM | 1565 | CG | PRO | B | 1 | 28.475 | 23.690 | 32.579 | 1.00 | 29.40 | C |
| ATOM | 1568 | CD | PRO | B | 1 | 29.057 | 22.297 | 32.717 | 1.00 | 28.94 | C |
| ATOM | 1571 | C | PRO | B | 1 | 25.884 | 22.312 | 33.816 | 1.00 | 28.05 | C |
| ATOM | 1572 | O | PRO | B | 1 | 25.750 | 21.643 | 32.792 | 1.00 | 28.57 | O |
| ATOM | 1575 | N | GLN | B | 2 | 24.860 | 22.788 | 34.512 | 1.00 | 27.60 | N |
| ATOM | 1576 | CA | GLN | B | 2 | 23.526 | 22.880 | 33.937 | 1.00 | 27.89 | C |
| ATOM | 1578 | CB | GLN | B | 2 | 22.468 | 22.272 | 34.856 | 1.00 | 28.32 | C |
| ATOM | 1581 | CG | GLN | B | 2 | 21.029 | 22.500 | 34.391 | 1.00 | 27.46 | C |
| ATOM | 1584 | CD | GLN | B | 2 | 20.022 | 21.666 | 35.166 | 1.00 | 27.66 | C |
| ATOM | 1585 | OE1 | GLN | B | 2 | 19.531 | 20.654 | 34.671 | 1.00 | 28.34 | O |
| ATOM | 1586 | NE2 | GLN | B | 2 | 19.712 | 22.088 | 36.386 | 1.00 | 25.23 | N |
| ATOM | 1589 | C | GLN | B | 2 | 23.239 | 24.351 | 33.708 | 1.00 | 28.46 | C |
| ATOM | 1590 | O | GLN | B | 2 | 23.479 | 25.191 | 34.587 | 1.00 | 28.11 | O |
| ATOM | 1592 | N | ILE | B | 3 | 22.745 | 24.651 | 32.516 | 1.00 | 28.24 | N |
| ATOM | 1593 | CA | ILE | B | 3 | 22.581 | 26.022 | 32.069 | 1.00 | 28.82 | C |
| ATOM | 1595 | CB | ILE | B | 3 | 23.526 | 26.348 | 30.891 | 1.00 | 28.96 | C |
| ATOM | 1597 | CG1 | ILE | B | 3 | 24.972 | 26.412 | 31.394 | 1.00 | 30.10 | C |
| ATOM | 1600 | CD1 | ILE | B | 3 | 26.009 | 26.094 | 30.342 | 1.00 | 29.49 | C |
| ATOM | 1604 | CG2 | ILE | B | 3 | 23.141 | 27.679 | 30.243 | 1.00 | 28.64 | C |
| ATOM | 1608 | C | ILE | B | 3 | 21.118 | 26.235 | 31.696 | 1.00 | 28.55 | C |
| ATOM | 1609 | O | ILE | B | 3 | 20.560 | 25.524 | 30.867 | 1.00 | 27.81 | O |
| ATOM | 1611 | N | THR | B | 4 | 20.485 | 27.168 | 32.391 | 1.00 | 28.37 | N |
| ATOM | 1612 | CA | THR | B | 4 | 19.111 | 27.525 | 32.103 | 1.00 | 28.33 | C |
| ATOM | 1614 | CB | THR | B | 4 | 18.438 | 28.167 | 33.323 | 1.00 | 28.34 | C |
| ATOM | 1616 | OG1 | THR | B | 4 | 19.109 | 29.394 | 33.632 | 1.00 | 26.36 | O |
| ATOM | 1618 | CG2 | THR | B | 4 | 18.515 | 27.219 | 34.518 | 1.00 | 29.48 | C |
| ATOM | 1622 | C | THR | B | 4 | 19.125 | 28.513 | 30.956 | 1.00 | 28.06 | C |
| ATOM | 1623 | O | THR | B | 4 | 20.190 | 28.979 | 30.549 | 1.00 | 28.37 | O |
| ATOM | 1625 | N | LEU | B | 5 | 17.939 | 28.863 | 30.473 | 1.00 | 27.91 | N |
| ATOM | 1626 | CA | LEU | B | 5 | 17.807 | 29.611 | 29.222 | 1.00 | 28.03 | C |
| ATOM | 1628 | CB | LEU | B | 5 | 17.099 | 28.739 | 28.188 | 1.00 | 28.04 | C |
| ATOM | 1631 | CG | LEU | B | 5 | 17.873 | 27.456 | 27.873 | 1.00 | 29.04 | C |
| ATOM | 1633 | CD1 | LEU | B | 5 | 17.085 | 26.506 | 26.951 | 1.00 | 29.52 | C |
| ATOM | 1637 | CD2 | LEU | B | 5 | 19.215 | 27.816 | 27.265 | 1.00 | 29.96 | C |
| ATOM | 1641 | C | LEU | B | 5 | 17.077 | 30.943 | 29.391 | 1.00 | 27.44 | C |
| ATOM | 1642 | O | LEU | B | 5 | 16.609 | 31.515 | 28.414 | 1.00 | 27.63 | O |
| ATOM | 1644 | N | TRP | B | 6 | 16.999 | 31.443 | 30.622 | 1.00 | 26.89 | N |
| ATOM | 1645 | CA | TRP | B | 6 | 16.424 | 32.766 | 30.884 | 1.00 | 26.59 | C |
| ATOM | 1647 | CB | TRP | B | 6 | 16.186 | 32.954 | 32.384 | 1.00 | 27.19 | C |
| ATOM | 1650 | CG | TRP | B | 6 | 15.289 | 31.878 | 32.926 | 1.00 | 26.57 | C |
| ATOM | 1651 | CD1 | TRP | B | 6 | 15.672 | 30.732 | 33.565 | 1.00 | 27.69 | C |
| ATOM | 1653 | NE1 | TRP | B | 6 | 14.570 | 29.966 | 33.868 | 1.00 | 28.54 | N |
| ATOM | 1655 | CE2 | TRP | B | 6 | 13.451 | 30.589 | 33.377 | 1.00 | 28.06 | C |
| ATOM | 1656 | CD2 | TRP | B | 6 | 13.868 | 31.793 | 32.770 | 1.00 | 26.83 | C |
| ATOM | 1657 | CE3 | TRP | B | 6 | 12.905 | 32.625 | 32.189 | 1.00 | 26.79 | C |
| ATOM | 1659 | CZ3 | TRP | B | 6 | 11.573 | 32.241 | 32.238 | 1.00 | 28.14 | C |
| ATOM | 1661 | CH2 | TRP | B | 6 | 11.189 | 31.039 | 32.854 | 1.00 | 28.36 | C |
| ATOM | 1663 | CZ2 | TRP | B | 6 | 12.114 | 30.198 | 33.422 | 1.00 | 28.52 | C |
| ATOM | 1665 | C | TRP | B | 6 | 17.316 | 33.870 | 30.302 | 1.00 | 26.56 | C |
| ATOM | 1666 | O | TRP | B | 6 | 16.858 | 34.994 | 30.036 | 1.00 | 26.50 | O |
| ATOM | 1668 | N | LYS | B | 7 | 18.591 | 33.543 | 30.132 | 1.00 | 24.96 | N |
| ATOM | 1669 | CA | LYS | B | 7 | 19.546 | 34.385 | 29.444 | 1.00 | 25.23 | C |
| ATOM | 1671 | CB | LYS | B | 7 | 20.684 | 34.789 | 30.393 | 1.00 | 26.35 | C |
| ATOM | 1678 | C | LYS | B | 7 | 20.133 | 33.585 | 28.292 | 1.00 | 25.95 | C |
| ATOM | 1679 | O | LYS | B | 7 | 20.016 | 32.358 | 28.252 | 1.00 | 27.34 | O |
| ATOM | 1681 | N | ARG | B | 8 | 20.778 | 34.279 | 27.362 | 1.00 | 25.12 | N |
| ATOM | 1682 | CA | ARG | B | 8 | 21.505 | 33.617 | 26.313 | 1.00 | 24.43 | C |
| ATOM | 1684 | CB | ARG | B | 8 | 22.153 | 34.659 | 25.403 | 1.00 | 24.24 | C |
| ATOM | 1687 | CG | ARG | B | 8 | 21.187 | 35.345 | 24.474 | 1.00 | 24.02 | C |
| ATOM | 1690 | CD | ARG | B | 8 | 21.857 | 36.487 | 23.719 | 1.00 | 24.77 | C |
| ATOM | 1693 | NE | ARG | B | 8 | 20.923 | 37.147 | 22.809 | 1.00 | 24.51 | N |
| ATOM | 1695 | CZ | ARG | B | 8 | 21.186 | 38.271 | 22.148 | 1.00 | 23.87 | C |
| ATOM | 1696 | NH1 | ARG | B | 8 | 22.350 | 38.885 | 22.302 | 1.00 | 25.00 | N |
| ATOM | 1699 | NH2 | ARG | B | 8 | 20.292 | 38.770 | 21.313 | 1.00 | 20.78 | N |
| ATOM | 1702 | C | ARG | B | 8 | 22.575 | 32.696 | 26.919 | 1.00 | 24.46 | C |
| ATOM | 1703 | O | ARG | B | 8 | 23.272 | 33.079 | 27.869 | 1.00 | 24.45 | O |
| ATOM | 1705 | N | PRO | B | 9 | 22.679 | 31.459 | 26.407 | 1.00 | 24.04 | N |
| ATOM | 1706 | CA | PRO | B | 9 | 23.700 | 30.538 | 26.898 | 1.00 | 25.01 | C |
| ATOM | 1708 | CB | PRO | B | 9 | 23.148 | 29.166 | 26.501 | 1.00 | 24.99 | C |
| ATOM | 1711 | CG | PRO | B | 9 | 22.367 | 29.438 | 25.283 | 1.00 | 24.35 | C |
| ATOM | 1714 | CD | PRO | B | 9 | 21.758 | 30.788 | 25.475 | 1.00 | 24.69 | C |
| ATOM | 1717 | C | PRO | B | 9 | 25.067 | 30.837 | 26.299 | 1.00 | 25.49 | C |
| ATOM | 1718 | O | PRO | B | 9 | 25.631 | 30.038 | 25.536 | 1.00 | 25.60 | O |
| ATOM | 1719 | N | LEU | B | 10 | 25.601 | 31.984 | 26.714 | 1.00 | 26.71 | N |
| ATOM | 1720 | CA | LEU | B | 10 | 26.931 | 32.436 | 26.318 | 1.00 | 27.77 | C |
| ATOM | 1722 | CB | LEU | B | 10 | 26.980 | 33.979 | 26.308 | 1.00 | 28.51 | C |
| ATOM | 1725 | CG | LEU | B | 10 | 26.088 | 34.700 | 25.282 | 1.00 | 28.93 | C |
| ATOM | 1727 | CD1 | LEU | B | 10 | 25.963 | 36.199 | 25.579 | 1.00 | 28.46 | C |
| ATOM | 1731 | CD2 | LEU | B | 10 | 26.640 | 34.482 | 23.871 | 1.00 | 30.59 | C |
| ATOM | 1735 | C | LEU | B | 10 | 27.959 | 31.878 | 27.310 | 1.00 | 27.61 | C |
| ATOM | 1736 | O | LEU | B | 10 | 27.780 | 31.957 | 28.520 | 1.00 | 27.85 | O |
| ATOM | 1738 | N | VAL | B | 11 | 29.024 | 31.280 | 26.800 | 1.00 | 27.30 | N |
| ATOM | 1739 | CA | VAL | B | 11 | 30.072 | 30.750 | 27.657 | 1.00 | 26.67 | C |
| ATOM | 1741 | CB | VAL | B | 11 | 30.044 | 29.205 | 27.714 | 1.00 | 26.87 | C |
| ATOM | 1743 | CG1 | VAL | B | 11 | 28.722 | 28.719 | 28.303 | 1.00 | 27.79 | C |
| ATOM | 1747 | CG2 | VAL | B | 11 | 30.285 | 28.598 | 26.329 | 1.00 | 27.11 | C |
| ATOM | 1751 | C | VAL | B | 11 | 31.388 | 31.214 | 27.075 | 1.00 | 25.80 | C |
| ATOM | 1752 | O | VAL | B | 11 | 31.434 | 31.671 | 25.938 | 1.00 | 25.13 | O |
| ATOM | 1754 | N | THR | B | 12 | 32.459 | 31.059 | 27.838 | 1.00 | 24.78 | N |
| ATOM | 1755 | CA | THR | B | 12 | 33.771 | 31.411 | 27.345 | 1.00 | 23.85 | C |
| ATOM | 1757 | CB | THR | B | 12 | 34.644 | 31.904 | 28.504 | 1.00 | 23.62 | C |
| ATOM | 1759 | OG1 | THR | B | 12 | 34.007 | 33.037 | 29.103 | 1.00 | 23.02 | O |
| ATOM | 1761 | CG2 | THR | B | 12 | 36.015 | 32.305 | 28.032 | 1.00 | 22.71 | C |
| ATOM | 1765 | C | THR | B | 12 | 34.391 | 30.190 | 26.656 | 1.00 | 24.43 | C |
| ATOM | 1766 | O | THR | B | 12 | 34.296 | 29.062 | 27.154 | 1.00 | 24.09 | O |
| ATOM | 1768 | N | ILE | B | 13 | 34.960 | 30.412 | 25.477 | 1.00 | 24.05 | N |
| ATOM | 1769 | CA | ILE | B | 13 | 35.683 | 29.364 | 24.771 | 1.00 | 24.52 | C |
| ATOM | 1771 | CB | ILE | B | 13 | 35.104 | 29.077 | 23.374 | 1.00 | 24.89 | C |
| ATOM | 1773 | CG1 | ILE | B | 13 | 35.312 | 30.295 | 22.456 | 1.00 | 25.17 | C |
| ATOM | 1776 | CD1 | ILE | B | 13 | 34.838 | 30.128 | 21.047 | 1.00 | 26.54 | C |
| ATOM | 1780 | CG2 | ILE | B | 13 | 33.630 | 28.675 | 23.486 | 1.00 | 23.41 | C |
| ATOM | 1784 | C | ILE | B | 13 | 37.131 | 29.791 | 24.639 | 1.00 | 25.13 | C |
| ATOM | 1785 | O | ILE | B | 13 | 37.460 | 30.979 | 24.699 | 1.00 | 24.49 | O |
| ATOM | 1787 | N | ARG | B | 14 | 38.009 | 28.815 | 24.483 | 1.00 | 25.91 | N |
| ATOM | 1788 | CA | ARG | B | 14 | 39.418 | 29.134 | 24.273 | 1.00 | 26.30 | C |
| ATOM | 1790 | CB | ARG | B | 14 | 40.278 | 28.616 | 25.427 | 1.00 | 25.93 | C |
| ATOM | 1793 | CG | ARG | B | 14 | 41.747 | 28.961 | 25.295 | 1.00 | 27.26 | C |
| ATOM | 1796 | CD | ARG | B | 14 | 42.453 | 28.829 | 26.632 | 1.00 | 28.76 | C |
| ATOM | 1799 | NE | ARG | B | 14 | 43.899 | 28.754 | 26.463 | 1.00 | 32.95 | N |
| ATOM | 1801 | CZ | ARG | B | 14 | 44.618 | 27.635 | 26.513 | 1.00 | 34.10 | C |
| ATOM | 1802 | NH1 | ARG | B | 14 | 44.038 | 26.461 | 26.737 | 1.00 | 36.10 | N |
| ATOM | 1805 | NH2 | ARG | B | 14 | 45.934 | 27.695 | 26.341 | 1.00 | 32.77 | N |
| ATOM | 1808 | C | ARG | B | 14 | 39.833 | 28.486 | 22.988 | 1.00 | 25.23 | C |
| ATOM | 1809 | O | ARG | B | 14 | 39.608 | 27.298 | 22.797 | 1.00 | 24.61 | O |
| ATOM | 1811 | N | ILE | B | 15 | 40.377 | 29.284 | 22.082 | 1.00 | 26.63 | N |
| ATOM | 1812 | CA | ILE | B | 15 | 40.813 | 28.764 | 20.798 | 1.00 | 27.11 | C |
| ATOM | 1814 | CB | ILE | B | 15 | 39.669 | 28.824 | 19.731 | 1.00 | 27.69 | C |
| ATOM | 1816 | CG1 | ILE | B | 15 | 40.121 | 28.190 | 18.421 | 1.00 | 27.99 | C |
| ATOM | 1819 | CD1 | ILE | B | 15 | 39.009 | 28.057 | 17.399 | 1.00 | 28.78 | C |
| ATOM | 1823 | CG2 | ILE | B | 15 | 39.155 | 30.252 | 19.510 | 1.00 | 28.84 | C |
| ATOM | 1827 | C | ILE | B | 15 | 42.088 | 29.492 | 20.375 | 1.00 | 26.60 | C |
| ATOM | 1828 | O | ILE | B | 15 | 42.152 | 30.719 | 20.383 | 1.00 | 26.55 | O |
| ATOM | 1830 | N | GLY | B | 16 | 43.140 | 28.723 | 20.115 | 1.00 | 26.55 | N |
| ATOM | 1831 | CA | GLY | B | 16 | 44.428 | 29.287 | 19.731 | 1.00 | 26.01 | C |
| ATOM | 1834 | C | GLY | B | 16 | 44.961 | 30.215 | 20.798 | 1.00 | 25.68 | C |
| ATOM | 1835 | O | GLY | B | 16 | 45.626 | 31.207 | 20.495 | 1.00 | 24.97 | O |
| ATOM | 1837 | N | GLY | B | 17 | 44.630 | 29.912 | 22.049 | 1.00 | 25.50 | N |
| ATOM | 1838 | CA | GLY | B | 17 | 45.029 | 30.748 | 23.171 | 1.00 | 25.69 | C |
| ATOM | 1841 | C | GLY | B | 17 | 44.321 | 32.088 | 23.196 | 1.00 | 25.78 | C |
| ATOM | 1842 | O | GLY | B | 17 | 44.793 | 33.012 | 23.841 | 1.00 | 26.31 | O |
| ATOM | 1844 | N | GLN | B | 18 | 43.211 | 32.194 | 22.473 | 1.00 | 25.86 | N |
| ATOM | 1845 | CA | GLN | B | 18 | 42.364 | 33.383 | 22.494 | 1.00 | 26.91 | C |
| ATOM | 1847 | CB | GLN | B | 18 | 42.009 | 33.822 | 21.072 | 1.00 | 27.45 | C |
| ATOM | 1850 | CG | GLN | B | 18 | 43.184 | 34.310 | 20.252 | 1.00 | 30.06 | C |
| ATOM | 1853 | CD | GLN | B | 18 | 43.531 | 35.761 | 20.538 | 1.00 | 33.86 | C |
| ATOM | 1854 | OE1 | GLN | B | 18 | 44.440 | 36.047 | 21.314 | 1.00 | 36.99 | O |
| ATOM | 1855 | NE2 | GLN | B | 18 | 42.799 | 36.685 | 19.918 | 1.00 | 34.85 | N |
| ATOM | 1858 | C | GLN | B | 18 | 41.088 | 33.055 | 23.254 | 1.00 | 26.27 | C |
| ATOM | 1859 | O | GLN | B | 18 | 40.501 | 31.989 | 23.034 | 1.00 | 26.90 | O |
| ATOM | 1861 | N | LEU | B | 19 | 40.675 | 33.953 | 24.151 | 1.00 | 25.35 | N |
| ATOM | 1862 | CA | LEU | B | 19 | 39.401 | 33.802 | 24.853 | 1.00 | 26.07 | C |
| ATOM | 1864 | CB | LEU | B | 19 | 39.506 | 34.294 | 26.297 | 1.00 | 26.74 | C |
| ATOM | 1867 | CG | LEU | B | 19 | 40.452 | 33.538 | 27.223 | 1.00 | 27.32 | C |
| ATOM | 1869 | CD1 | LEU | B | 19 | 40.395 | 34.119 | 28.632 | 1.00 | 28.10 | C |
| ATOM | 1873 | CD2 | LEU | B | 19 | 40.097 | 32.068 | 27.222 | 1.00 | 28.30 | C |
| ATOM | 1877 | C | LEU | B | 19 | 38.301 | 34.557 | 24.132 | 1.00 | 25.85 | C |
| ATOM | 1878 | O | LEU | B | 19 | 38.457 | 35.733 | 23.800 | 1.00 | 25.40 | O |
| ATOM | 1880 | N | LYS | B | 20 | 37.187 | 33.873 | 23.887 | 1.00 | 26.54 | N |
| ATOM | 1881 | CA | LYS | B | 20 | 36.026 | 34.481 | 23.263 | 1.00 | 26.29 | C |
| ATOM | 1883 | CB | LYS | B | 20 | 35.994 | 34.151 | 21.776 | 1.00 | 26.29 | C |
| ATOM | 1886 | CG | LYS | B | 20 | 37.307 | 34.494 | 21.088 | 1.00 | 28.35 | C |
| ATOM | 1889 | CD | LYS | B | 20 | 37.226 | 34.344 | 19.593 | 1.00 | 28.37 | C |
| ATOM | 1892 | CE | LYS | B | 20 | 38.319 | 35.174 | 18.911 | 1.00 | 30.67 | C |
| ATOM | 1895 | NZ | LYS | B | 20 | 37.983 | 35.416 | 17.508 | 1.00 | 28.64 | N |
| ATOM | 1899 | C | LYS | B | 20 | 34.750 | 34.002 | 23.931 | 1.00 | 25.66 | C |
| ATOM | 1900 | O | LYS | B | 20 | 34.748 | 32.991 | 24.605 | 1.00 | 26.11 | O |
| ATOM | 1902 | N | GLU | B | 21 | 33.670 | 34.749 | 23.731 | 1.00 | 25.00 | N |
| ATOM | 1903 | CA | GLU | B | 21 | 32.339 | 34.338 | 24.152 | 1.00 | 25.47 | C |
| ATOM | 1905 | CB | GLU | B | 21 | 31.559 | 35.523 | 24.735 | 1.00 | 24.64 | C |
| ATOM | 1908 | CG | GLU | B | 21 | 32.245 | 36.164 | 25.928 | 1.00 | 27.60 | C |
| ATOM | 1911 | CD | GLU | B | 21 | 32.381 | 35.215 | 27.124 | 1.00 | 29.65 | C |
| ATOM | 1912 | OE1 | GLU | B | 21 | 31.348 | 34.700 | 27.629 | 1.00 | 31.36 | O |
| ATOM | 1913 | OE2 | GLU | B | 21 | 33.531 | 34.989 | 27.546 | 1.00 | 26.07 | O |
| ATOM | 1914 | C | GLU | B | 21 | 31.594 | 33.757 | 22.957 | 1.00 | 25.41 | C |
| ATOM | 1915 | O | GLU | B | 21 | 31.658 | 34.278 | 21.845 | 1.00 | 23.60 | O |
| ATOM | 1917 | N | ALA | B | 22 | 30.914 | 32.650 | 23.197 | 1.00 | 24.98 | N |
| ATOM | 1918 | CA | ALA | B | 22 | 30.143 | 32.009 | 22.155 | 1.00 | 26.35 | C |
| ATOM | 1920 | CB | ALA | B | 22 | 30.953 | 30.885 | 21.503 | 1.00 | 26.49 | C |
| ATOM | 1924 | C | ALA | B | 22 | 28.869 | 31.451 | 22.728 | 1.00 | 27.06 | C |
| ATOM | 1925 | O | ALA | B | 22 | 28.804 | 31.088 | 23.907 | 1.00 | 27.35 | O |
| ATOM | 1927 | N | LEU | B | 23 | 27.910 | 31.265 | 21.832 | 1.00 | 27.27 | N |
| ATOM | 1928 | CA | LEU | B | 23 | 26.555 | 30.866 | 22.173 | 1.00 | 27.22 | C |
| ATOM | 1930 | CB | LEU | B | 23 | 25.617 | 31.647 | 21.252 | 1.00 | 27.68 | C |
| ATOM | 1933 | CG | LEU | B | 23 | 24.109 | 31.543 | 21.359 | 1.00 | 29.22 | C |
| ATOM | 1935 | CD1 | LEU | B | 23 | 23.616 | 32.223 | 22.610 | 1.00 | 30.66 | C |
| ATOM | 1939 | CD2 | LEU | B | 23 | 23.505 | 32.160 | 20.087 | 1.00 | 28.12 | C |
| ATOM | 1943 | C | LEU | B | 23 | 26.386 | 29.359 | 21.963 | 1.00 | 26.49 | C |
| ATOM | 1944 | O | LEU | B | 23 | 26.656 | 28.842 | 20.880 | 1.00 | 27.87 | O |
| ATOM | 1946 | N | LEU | B | 24 | 25.955 | 28.651 | 23.004 | 1.00 | 26.35 | N |
| ATOM | 1947 | CA | LEU | B | 24 | 25.646 | 27.220 | 22.899 | 1.00 | 25.61 | C |
| ATOM | 1949 | CB | LEU | B | 24 | 25.632 | 26.572 | 24.284 | 1.00 | 26.50 | C |
| ATOM | 1952 | CG | LEU | B | 24 | 26.911 | 26.664 | 25.126 | 1.00 | 24.10 | C |
| ATOM | 1954 | CD1 | LEU | B | 24 | 26.736 | 25.914 | 26.424 | 1.00 | 25.83 | C |
| ATOM | 1958 | CD2 | LEU | B | 24 | 28.132 | 26.160 | 24.396 | 1.00 | 26.37 | C |
| ATOM | 1962 | C | LEU | B | 24 | 24.295 | 27.050 | 22.201 | 1.00 | 25.78 | C |
| ATOM | 1963 | O | LEU | B | 24 | 23.267 | 27.458 | 22.737 | 1.00 | 27.17 | O |
| ATOM | 1965 | N | ASP | B | 25 | 24.319 | 26.524 | 20.975 | 1.00 | 25.61 | N |
| ATOM | 1966 | CA | ASP | B | 25 | 23.195 | 26.626 | 20.043 | 1.00 | 26.34 | C |
| ATOM | 1968 | CB | ASP | B | 25 | 23.528 | 27.601 | 18.901 | 1.00 | 26.22 | C |
| ATOM | 1971 | CG | ASP | B | 25 | 22.327 | 27.927 | 18.032 | 1.00 | 27.28 | C |
| ATOM | 1972 | OD1 | ASP | B | 25 | 21.252 | 27.296 | 18.178 | 1.00 | 29.52 | O |
| ATOM | 1973 | OD2 | ASP | B | 25 | 22.456 | 28.821 | 17.179 | 1.00 | 25.26 | O |
| ATOM | 1974 | C | ASP | B | 25 | 22.842 | 25.262 | 19.454 | 1.00 | 26.02 | C |
| ATOM | 1975 | O | ASP | B | 25 | 23.445 | 24.798 | 18.472 | 1.00 | 25.92 | O |
| ATOM | 1977 | N | THR | B | 26 | 21.804 | 24.663 | 20.027 | 1.00 | 25.67 | N |
| ATOM | 1978 | CA | THR | B | 26 | 21.338 | 23.370 | 19.584 | 1.00 | 25.45 | C |
| ATOM | 1980 | CB | THR | B | 26 | 20.358 | 22.796 | 20.604 | 1.00 | 24.04 | C |
| ATOM | 1982 | OG1 | THR | B | 26 | 19.247 | 23.686 | 20.766 | 1.00 | 23.15 | O |
| ATOM | 1984 | CG2 | THR | B | 26 | 21.072 | 22.599 | 21.918 | 1.00 | 25.50 | C |
| ATOM | 1988 | C | THR | B | 26 | 20.672 | 23.435 | 18.211 | 1.00 | 25.94 | C |
| ATOM | 1989 | O | THR | B | 26 | 20.456 | 22.394 | 17.576 | 1.00 | 25.85 | O |
| ATOM | 1991 | N | GLY | B | 27 | 20.333 | 24.645 | 17.764 | 1.00 | 25.94 | N |
| ATOM | 1992 | CA | GLY | B | 27 | 19.715 | 24.825 | 16.461 | 1.00 | 26.60 | C |
| ATOM | 1995 | C | GLY | B | 27 | 20.696 | 24.896 | 15.302 | 1.00 | 26.59 | C |
| ATOM | 1996 | O | GLY | B | 27 | 20.286 | 24.949 | 14.142 | 1.00 | 27.90 | O |
| ATOM | 1998 | N | ALA | B | 28 | 21.985 | 24.982 | 15.608 | 1.00 | 26.45 | N |
| ATOM | 1999 | CA | ALA | B | 28 | 23.030 | 25.026 | 14.589 | 1.00 | 25.92 | C |
| ATOM | 2001 | CB | ALA | B | 28 | 24.056 | 26.074 | 14.962 | 1.00 | 25.79 | C |
| ATOM | 2005 | C | ALA | B | 28 | 23.682 | 23.643 | 14.485 | 1.00 | 26.63 | C |
| ATOM | 2006 | O | ALA | B | 28 | 24.034 | 23.037 | 15.498 | 1.00 | 26.87 | O |
| ATOM | 2008 | N | ASP | B | 29 | 23.799 | 23.144 | 13.262 | 1.00 | 27.35 | N |
| ATOM | 2009 | CA | ASP | B | 29 | 24.498 | 21.886 | 12.974 | 1.00 | 27.62 | C |
| ATOM | 2011 | CB | ASP | B | 29 | 24.270 | 21.448 | 11.525 | 1.00 | 27.64 | C |
| ATOM | 2014 | CG | ASP | B | 29 | 22.801 | 21.337 | 11.141 | 1.00 | 29.65 | C |
| ATOM | 2015 | OD1 | ASP | B | 29 | 21.935 | 21.179 | 12.029 | 1.00 | 30.03 | O |
| ATOM | 2016 | OD2 | ASP | B | 29 | 22.527 | 21.367 | 9.918 | 1.00 | 25.22 | O |
| ATOM | 2017 | C | ASP | B | 29 | 26.003 | 22.066 | 13.148 | 1.00 | 27.05 | C |
| ATOM | 2018 | O | ASP | B | 29 | 26.712 | 21.133 | 13.526 | 1.00 | 26.40 | O |
| ATOM | 2020 | N | ASP | B | 30 | 26.452 | 23.287 | 12.861 | 1.00 | 26.67 | N |
| ATOM | 2021 | CA | ASP | B | 30 | 27.842 | 23.639 | 12.631 | 1.00 | 26.85 | C |
| ATOM | 2023 | CB | ASP | B | 30 | 27.977 | 24.363 | 11.282 | 1.00 | 27.59 | C |
| ATOM | 2026 | CG | ASP | B | 30 | 27.673 | 23.496 | 10.117 | 1.00 | 27.17 | C |
| ATOM | 2027 | OD1 | ASP | B | 30 | 28.153 | 22.355 | 10.099 | 1.00 | 28.05 | O |
| ATOM | 2028 | OD2 | ASP | B | 30 | 26.999 | 23.989 | 9.193 | 1.00 | 33.50 | O |
| ATOM | 2029 | C | ASP | B | 30 | 28.237 | 24.687 | 13.648 | 1.00 | 26.50 | C |
| ATOM | 2030 | O | ASP | B | 30 | 27.390 | 25.427 | 14.147 | 1.00 | 27.13 | O |
| ATOM | 2032 | N | THR | B | 31 | 29.538 | 24.862 | 13.781 | 1.00 | 26.42 | N |
| ATOM | 2033 | CA | THR | B | 31 | 30.120 | 25.881 | 14.654 | 1.00 | 25.75 | C |
| ATOM | 2035 | CB | THR | B | 31 | 31.215 | 25.253 | 15.518 | 1.00 | 24.10 | C |
| ATOM | 2037 | OG1 | THR | B | 31 | 30.601 | 24.401 | 16.499 | 1.00 | 24.16 | O |
| ATOM | 2039 | CG2 | THR | B | 31 | 32.072 | 26.308 | 16.216 | 1.00 | 24.29 | C |
| ATOM | 2043 | C | THR | B | 31 | 30.652 | 27.004 | 13.765 | 1.00 | 25.64 | C |
| ATOM | 2044 | O | THR | B | 31 | 31.403 | 26.746 | 12.821 | 1.00 | 25.01 | O |
| ATOM | 2046 | N | VAL | B | 32 | 30.194 | 28.230 | 14.030 | 1.00 | 26.39 | N |
| ATOM | 2047 | CA | VAL | B | 32 | 30.535 | 29.395 | 13.205 | 1.00 | 26.94 | C |
| ATOM | 2049 | CB | VAL | B | 32 | 29.331 | 29.893 | 12.356 | 1.00 | 26.72 | C |
| ATOM | 2051 | CG1 | VAL | B | 32 | 29.788 | 30.889 | 11.314 | 1.00 | 26.37 | C |
| ATOM | 2055 | CG2 | VAL | B | 32 | 28.614 | 28.729 | 11.689 | 1.00 | 28.34 | C |
| ATOM | 2059 | C | VAL | B | 32 | 31.025 | 30.532 | 14.105 | 1.00 | 26.69 | C |
| ATOM | 2060 | O | VAL | B | 32 | 30.291 | 31.026 | 14.957 | 1.00 | 26.37 | O |
| ATOM | 2062 | N | LEU | B | 33 | 32.277 | 30.937 | 13.898 | 1.00 | 27.60 | N |
| ATOM | 2063 | CA | LEU | B | 33 | 32.918 | 31.986 | 14.681 | 1.00 | 26.53 | C |
| ATOM | 2065 | CB | LEU | B | 33 | 34.242 | 31.479 | 15.282 | 1.00 | 26.28 | C |
| ATOM | 2068 | CG | LEU | B | 33 | 34.165 | 30.258 | 16.209 | 1.00 | 26.88 | C |
| ATOM | 2070 | CD1 | LEU | B | 33 | 35.527 | 29.954 | 16.804 | 1.00 | 27.98 | C |
| ATOM | 2074 | CD2 | LEU | B | 33 | 33.165 | 30.501 | 17.334 | 1.00 | 24.90 | C |
| ATOM | 2078 | C | LEU | B | 33 | 33.195 | 33.208 | 13.804 | 1.00 | 26.64 | C |
| ATOM | 2079 | O | LEU | B | 33 | 33.432 | 33.078 | 12.606 | 1.00 | 27.24 | O |
| ATOM | 2081 | N | GLU | B | 34 | 33.163 | 34.382 | 14.425 | 1.00 | 26.07 | N |
| ATOM | 2082 | CA | GLU | B | 34 | 33.475 | 35.655 | 13.784 | 1.00 | 26.23 | C |
| ATOM | 2084 | CB | GLU | B | 34 | 33.405 | 36.796 | 14.805 | 1.00 | 25.97 | C |
| ATOM | 2087 | CG | GLU | B | 34 | 32.051 | 36.950 | 15.475 | 1.00 | 28.39 | C |
| ATOM | 2090 | CD | GLU | B | 34 | 32.105 | 37.759 | 16.760 | 1.00 | 27.69 | C |
| ATOM | 2091 | OE1 | GLU | B | 34 | 33.218 | 38.084 | 17.241 | 1.00 | 30.00 | O |
| ATOM | 2092 | OE2 | GLU | B | 34 | 31.022 | 38.035 | 17.307 | 1.00 | 31.59 | O |
| ATOM | 2093 | C | GLU | B | 34 | 34.880 | 35.631 | 13.204 | 1.00 | 26.05 | C |
| ATOM | 2094 | O | GLU | B | 34 | 35.688 | 34.754 | 13.521 | 1.00 | 25.50 | O |
| ATOM | 2096 | N | GLU | B | 35 | 35.169 | 36.610 | 12.350 | 1.00 | 26.10 | N |
| ATOM | 2097 | CA | GLU | B | 35 | 36.421 | 36.621 | 11.617 | 1.00 | 26.47 | C |
| ATOM | 2099 | CB | GLU | B | 35 | 36.548 | 37.894 | 10.778 | 1.00 | 26.61 | C |
| ATOM | 2102 | CG | GLU | B | 35 | 37.259 | 37.663 | 9.468 | 1.00 | 28.97 | C |
| ATOM | 2105 | CD | GLU | B | 35 | 36.542 | 36.625 | 8.620 | 1.00 | 30.96 | C |
| ATOM | 2106 | OE1 | GLU | B | 35 | 35.299 | 36.497 | 8.718 | 1.00 | 30.65 | O |
| ATOM | 2107 | OE2 | GLU | B | 35 | 37.235 | 35.911 | 7.880 | 1.00 | 33.64 | O |
| ATOM | 2108 | C | GLU | B | 35 | 37.609 | 36.506 | 12.563 | 1.00 | 26.29 | C |
| ATOM | 2109 | O | GLU | B | 35 | 37.685 | 37.214 | 13.567 | 1.00 | 26.48 | O |
| ATOM | 2111 | N | MET | B | 36 | 38.536 | 35.618 | 12.223 | 1.00 | 25.75 | N |
| ATOM | 2112 | CA | MET | B | 36 | 39.747 | 35.399 | 13.015 | 1.00 | 27.09 | C |
| ATOM | 2114 | CB | MET | B | 36 | 39.448 | 34.620 | 14.291 | 1.00 | 26.63 | C |
| ATOM | 2117 | CG | MET | B | 36 | 38.931 | 33.205 | 14.025 | 1.00 | 27.93 | C |
| ATOM | 2120 | SD | MET | B | 36 | 38.683 | 32.276 | 15.542 | 1.00 | 29.64 | S |
| ATOM | 2121 | CE | MET | B | 36 | 40.351 | 32.254 | 16.193 | 1.00 | 28.88 | C |
| ATOM | 2125 | C | MET | B | 36 | 40.717 | 34.595 | 12.168 | 1.00 | 26.57 | C |
| ATOM | 2126 | O | MET | B | 36 | 40.306 | 33.895 | 11.246 | 1.00 | 27.04 | O |
| ATOM | 2128 | N | ASN | B | 37 | 42.001 | 34.713 | 12.471 | 1.00 | 26.81 | N |
| ATOM | 2129 | CA | ASN | B | 37 | 43.012 | 33.923 | 11.791 | 1.00 | 26.83 | C |
| ATOM | 2131 | CB | ASN | B | 37 | 44.270 | 34.753 | 11.578 | 1.00 | 26.86 | C |
| ATOM | 2134 | CG | ASN | B | 37 | 44.036 | 35.894 | 10.620 | 1.00 | 26.61 | C |
| ATOM | 2135 | OD1 | ASN | B | 37 | 44.414 | 37.032 | 10.886 | 1.00 | 27.12 | O |
| ATOM | 2136 | ND2 | ASN | B | 37 | 43.360 | 35.602 | 9.516 | 1.00 | 25.12 | N |
| ATOM | 2139 | C | ASN | B | 37 | 43.318 | 32.655 | 12.566 | 1.00 | 27.63 | C |
| ATOM | 2140 | O | ASN | B | 37 | 43.761 | 32.712 | 13.713 | 1.00 | 28.34 | O |
| ATOM | 2142 | N | LEU | B | 38 | 43.010 | 31.516 | 11.952 | 1.00 | 28.09 | N |
| ATOM | 2143 | CA | LEU | B | 38 | 43.426 | 30.216 | 12.463 | 1.00 | 28.41 | C |
| ATOM | 2145 | CB | LEU | B | 38 | 42.219 | 29.270 | 12.510 | 1.00 | 28.75 | C |
| ATOM | 2148 | CG | LEU | B | 38 | 41.084 | 29.646 | 13.467 | 1.00 | 29.30 | C |
| ATOM | 2150 | CD1 | LEU | B | 38 | 39.816 | 28.777 | 13.257 | 1.00 | 29.77 | C |
| ATOM | 2154 | CD2 | LEU | B | 38 | 41.569 | 29.518 | 14.898 | 1.00 | 29.65 | C |
| ATOM | 2158 | C | LEU | B | 38 | 44.522 | 29.661 | 11.545 | 1.00 | 28.34 | C |
| ATOM | 2159 | O | LEU | B | 38 | 44.556 | 29.984 | 10.354 | 1.00 | 27.90 | O |
| ATOM | 2161 | N | PRO | B | 39 | 45.439 | 28.848 | 12.094 | 1.00 | 28.53 | N |
| ATOM | 2162 | CA | PRO | B | 39 | 46.499 | 28.284 | 11.263 | 1.00 | 28.82 | C |
| ATOM | 2164 | CB | PRO | B | 39 | 47.559 | 27.833 | 12.286 | 1.00 | 28.74 | C |
| ATOM | 2167 | CG | PRO | B | 39 | 47.046 | 28.255 | 13.640 | 1.00 | 28.92 | C |
| ATOM | 2170 | CD | PRO | B | 39 | 45.571 | 28.410 | 13.491 | 1.00 | 28.54 | C |
| ATOM | 2173 | C | PRO | B | 39 | 46.024 | 27.091 | 10.446 | 1.00 | 28.79 | C |
| ATOM | 2174 | O | PRO | B | 39 | 44.987 | 26.506 | 10.745 | 1.00 | 28.95 | O |
| ATOM | 2175 | N | GLY | B | 40 | 46.801 | 26.724 | 9.434 | 1.00 | 29.25 | N |
| ATOM | 2176 | CA | GLY | B | 40 | 46.522 | 25.529 | 8.648 | 1.00 | 29.37 | C |
| ATOM | 2179 | C | GLY | B | 40 | 45.665 | 25.830 | 7.434 | 1.00 | 29.12 | C |
| ATOM | 2180 | O | GLY | B | 40 | 45.217 | 26.956 | 7.236 | 1.00 | 28.93 | O |
| ATOM | 2182 | N | ARG | B | 41 | 45.476 | 24.820 | 6.593 | 1.00 | 29.31 | N |
| ATOM | 2183 | CA | ARG | B | 41 | 44.709 | 24.995 | 5.372 | 1.00 | 29.26 | C |
| ATOM | 2185 | CB | ARG | B | 41 | 44.826 | 23.753 | 4.479 | 1.00 | 29.55 | C |
| ATOM | 2194 | C | ARG | B | 41 | 43.253 | 25.263 | 5.739 | 1.00 | 29.05 | C |
| ATOM | 2195 | O | ARG | B | 41 | 42.766 | 24.832 | 6.789 | 1.00 | 29.31 | O |
| ATOM | 2197 | N | TRP | B | 42 | 42.575 | 26.040 | 4.907 | 1.00 | 28.38 | N |
| ATOM | 2198 | CA | TRP | B | 42 | 41.138 | 26.153 | 5.013 | 1.00 | 27.74 | C |
| ATOM | 2200 | CB | TRP | B | 42 | 40.721 | 27.516 | 5.578 | 1.00 | 27.80 | C |
| ATOM | 2203 | CG | TRP | B | 42 | 41.326 | 28.677 | 4.866 | 1.00 | 28.14 | C |
| ATOM | 2204 | CD1 | TRP | B | 42 | 42.529 | 29.266 | 5.124 | 1.00 | 27.55 | C |
| ATOM | 2206 | NE1 | TRP | B | 42 | 42.736 | 30.311 | 4.262 | 1.00 | 27.75 | N |
| ATOM | 2208 | CE2 | TRP | B | 42 | 41.657 | 30.416 | 3.424 | 1.00 | 27.34 | C |
| ATOM | 2209 | CD2 | TRP | B | 42 | 40.746 | 29.403 | 3.780 | 1.00 | 27.25 | C |
| ATOM | 2210 | CE3 | TRP | B | 42 | 39.544 | 29.293 | 3.071 | 1.00 | 27.83 | C |
| ATOM | 2212 | CZ3 | TRP | B | 42 | 39.291 | 30.189 | 2.055 | 1.00 | 27.07 | C |
| ATOM | 2214 | CH2 | TRP | B | 42 | 40.219 | 31.182 | 1.719 | 1.00 | 27.79 | C |
| ATOM | 2216 | CZ2 | TRP | B | 42 | 41.407 | 31.312 | 2.391 | 1.00 | 27.58 | C |
| ATOM | 2218 | C | TRP | B | 42 | 40.563 | 25.924 | 3.634 | 1.00 | 27.26 | C |
| ATOM | 2219 | O | TRP | B | 42 | 41.276 | 26.024 | 2.631 | 1.00 | 27.35 | O |
| ATOM | 2221 | N | LYS | B | 43 | 39.292 | 25.552 | 3.606 | 1.00 | 26.99 | N |
| ATOM | 2222 | CA | LYS | B | 43 | 38.532 | 25.427 | 2.377 | 1.00 | 26.76 | C |
| ATOM | 2224 | CB | LYS | B | 43 | 37.879 | 24.039 | 2.298 | 1.00 | 27.06 | C |
| ATOM | 2227 | CG | LYS | B | 43 | 38.756 | 22.911 | 2.842 | 1.00 | 27.96 | C |
| ATOM | 2230 | CD | LYS | B | 43 | 38.272 | 21.507 | 2.442 | 1.00 | 27.65 | C |
| ATOM | 2233 | CE | LYS | B | 43 | 39.379 | 20.465 | 2.670 | 1.00 | 29.03 | C |
| ATOM | 2236 | NZ | LYS | B | 43 | 39.193 | 19.151 | 1.976 | 1.00 | 28.05 | N |
| ATOM | 2240 | C | LYS | B | 43 | 37.460 | 26.513 | 2.375 | 1.00 | 26.19 | C |
| ATOM | 2241 | O | LYS | B | 43 | 36.813 | 26.747 | 3.397 | 1.00 | 25.81 | O |
| ATOM | 2243 | N | PRO | B | 44 | 37.271 | 27.193 | 1.228 | 1.00 | 25.62 | N |
| ATOM | 2244 | CA | PRO | B | 44 | 36.096 | 28.047 | 1.091 | 1.00 | 25.50 | C |
| ATOM | 2246 | CB | PRO | B | 44 | 36.197 | 28.600 | −0.338 | 1.00 | 25.07 | C |
| ATOM | 2249 | CG | PRO | B | 44 | 37.552 | 28.283 | −0.805 | 1.00 | 25.92 | C |
| ATOM | 2252 | CD | PRO | B | 44 | 38.103 | 27.170 | 0.013 | 1.00 | 25.23 | C |
| ATOM | 2255 | C | PRO | B | 44 | 34.845 | 27.202 | 1.206 | 1.00 | 25.40 | C |
| ATOM | 2256 | O | PRO | B | 44 | 34.807 | 26.107 | 0.667 | 1.00 | 25.45 | O |
| ATOM | 2257 | N | LYS | B | 45 | 33.830 | 27.712 | 1.886 | 1.00 | 26.29 | N |
| ATOM | 2258 | CA | LYS | B | 45 | 32.562 | 27.014 | 2.011 | 1.00 | 27.11 | C |
| ATOM | 2260 | CB | LYS | B | 45 | 32.530 | 26.223 | 3.317 | 1.00 | 27.53 | C |
| ATOM | 2263 | CG | LYS | B | 45 | 31.411 | 25.205 | 3.395 | 1.00 | 29.20 | C |
| ATOM | 2266 | CD | LYS | B | 45 | 31.144 | 24.771 | 4.832 | 1.00 | 29.40 | C |
| ATOM | 2269 | CE | LYS | B | 45 | 29.883 | 23.930 | 4.921 | 1.00 | 29.45 | C |
| ATOM | 2272 | NZ | LYS | B | 45 | 30.088 | 22.546 | 4.450 | 1.00 | 29.75 | N |
| ATOM | 2276 | C | LYS | B | 45 | 31.439 | 28.036 | 1.986 | 1.00 | 27.03 | C |
| ATOM | 2277 | O | LYS | B | 45 | 31.617 | 29.170 | 2.431 | 1.00 | 27.15 | O |
| ATOM | 2279 | N | AMET | B | 46 | 30.297 | 27.661 | 1.420 | 0.50 | 27.52 | N |
| ATOM | 2280 | N | BMET | B | 46 | 30.289 | 27.630 | 1.459 | 0.50 | 26.90 | N |
| ATOM | 2281 | CA | AMET | B | 46 | 29.102 | 28.493 | 1.525 | 0.50 | 28.13 | C |
| ATOM | 2282 | CA | BMET | B | 46 | 29.091 | 28.459 | 1.499 | 0.50 | 26.81 | C |
| ATOM | 2285 | CB | AMET | B | 46 | 28.512 | 28.797 | 0.147 | 0.50 | 28.30 | C |
| ATOM | 2286 | CB | BMET | B | 46 | 28.525 | 28.631 | 0.089 | 0.50 | 26.91 | C |
| ATOM | 2291 | CG | AMET | B | 46 | 29.308 | 29.824 | −0.649 | 0.50 | 29.38 | C |
| ATOM | 2292 | CG | BMET | B | 46 | 29.519 | 29.226 | −0.912 | 0.50 | 26.55 | C |
| ATOM | 2297 | SD | AMET | B | 46 | 28.371 | 30.538 | −2.017 | 0.50 | 30.92 | S |
| ATOM | 2298 | SD | BMET | B | 46 | 29.962 | 30.949 | −0.592 | 0.50 | 25.23 | S |
| ATOM | 2299 | CE | AMET | B | 46 | 27.322 | 31.697 | −1.137 | 0.50 | 31.51 | C |
| ATOM | 2300 | CE | BMET | B | 46 | 28.366 | 31.731 | −0.380 | 0.50 | 23.94 | C |
| ATOM | 2307 | C | AMET | B | 46 | 28.068 | 27.800 | 2.397 | 0.50 | 27.11 | C |
| ATOM | 2308 | C | BMET | B | 46 | 28.057 | 27.792 | 2.395 | 0.50 | 26.47 | C |
| ATOM | 2309 | O | AMET | B | 46 | 27.800 | 26.620 | 2.224 | 0.50 | 26.72 | O |
| ATOM | 2310 | O | BMET | B | 46 | 27.777 | 26.613 | 2.236 | 0.50 | 26.08 | O |
| ATOM | 2313 | N | ILE | B | 47 | 27.502 | 28.543 | 3.343 | 1.00 | 26.75 | N |
| ATOM | 2314 | CA | ILE | B | 47 | 26.464 | 28.022 | 4.227 | 1.00 | 26.18 | C |
| ATOM | 2316 | CB | ILE | B | 47 | 26.972 | 27.894 | 5.685 | 1.00 | 26.69 | C |
| ATOM | 2318 | CG1 | ILE | B | 47 | 27.358 | 29.261 | 6.274 | 1.00 | 27.02 | C |
| ATOM | 2321 | CD1 | ILE | B | 47 | 27.759 | 29.177 | 7.733 | 1.00 | 27.26 | C |
| ATOM | 2325 | CG2 | ILE | B | 47 | 28.136 | 26.926 | 5.754 | 1.00 | 25.89 | C |
| ATOM | 2329 | C | ILE | B | 47 | 25.221 | 28.913 | 4.180 | 1.00 | 25.31 | C |
| ATOM | 2330 | O | ILE | B | 47 | 25.310 | 30.126 | 4.007 | 1.00 | 23.17 | O |
| ATOM | 2332 | N | GLY | B | 48 | 24.057 | 28.297 | 4.314 | 1.00 | 24.74 | N |
| ATOM | 2333 | CA | GLY | B | 48 | 22.804 | 29.038 | 4.261 | 1.00 | 25.92 | C |
| ATOM | 2336 | C | GLY | B | 48 | 22.178 | 29.169 | 5.627 | 1.00 | 25.71 | C |
| ATOM | 2337 | O | GLY | B | 48 | 22.387 | 28.320 | 6.496 | 1.00 | 26.95 | O |
| ATOM | 2339 | N | GLY | B | 49 | 21.377 | 30.211 | 5.806 | 1.00 | 25.41 | N |
| ATOM | 2340 | CA | GLY | B | 49 | 20.525 | 30.303 | 6.982 | 1.00 | 25.18 | C |
| ATOM | 2343 | C | GLY | B | 49 | 19.440 | 31.346 | 6.821 | 1.00 | 24.70 | C |
| ATOM | 2344 | O | GLY | B | 49 | 19.292 | 31.941 | 5.748 | 1.00 | 24.48 | O |
| ATOM | 2346 | N | ILE | B | 50 | 18.692 | 31.554 | 7.901 | 1.00 | 24.00 | N |
| ATOM | 2347 | CA | ILE | B | 50 | 17.767 | 32.680 | 8.020 | 1.00 | 23.79 | C |
| ATOM | 2349 | CB | ILE | B | 50 | 17.137 | 32.737 | 9.434 | 1.00 | 23.46 | C |
| ATOM | 2351 | CG1 | ILE | B | 50 | 15.900 | 31.836 | 9.480 | 1.00 | 23.01 | C |
| ATOM | 2354 | CD1 | ILE | B | 50 | 15.094 | 31.940 | 10.755 | 1.00 | 23.01 | C |
| ATOM | 2358 | CG2 | ILE | B | 50 | 16.776 | 34.172 | 9.827 | 1.00 | 24.09 | C |
| ATOM | 2362 | C | ILE | B | 50 | 18.489 | 33.989 | 7.674 | 1.00 | 23.68 | C |
| ATOM | 2363 | O | ILE | B | 50 | 19.481 | 34.356 | 8.307 | 1.00 | 24.46 | O |
| ATOM | 2365 | N | GLY | B | 51 | 18.009 | 34.658 | 6.631 | 1.00 | 23.47 | N |
| ATOM | 2366 | CA | GLY | B | 51 | 18.723 | 35.776 | 6.043 | 1.00 | 23.28 | C |
| ATOM | 2369 | C | GLY | B | 51 | 19.245 | 35.417 | 4.666 | 1.00 | 23.30 | C |
| ATOM | 2370 | O | GLY | B | 51 | 18.968 | 36.115 | 3.699 | 1.00 | 24.68 | O |
| ATOM | 2372 | N | GLY | B | 52 | 19.975 | 34.315 | 4.563 | 1.00 | 23.16 | N |
| ATOM | 2373 | CA | GLY | B | 52 | 20.556 | 33.907 | 3.281 | 1.00 | 23.10 | C |
| ATOM | 2376 | C | GLY | B | 52 | 21.826 | 33.068 | 3.380 | 1.00 | 22.81 | C |
| ATOM | 2377 | O | GLY | B | 52 | 22.090 | 32.442 | 4.410 | 1.00 | 23.46 | O |
| ATOM | 2379 | N | PHE | B | 53 | 22.585 | 33.050 | 2.284 | 1.00 | 21.96 | N |
| ATOM | 2380 | CA | PHE | B | 53 | 23.777 | 32.218 | 2.126 | 1.00 | 22.17 | C |
| ATOM | 2382 | CB | PHE | B | 53 | 23.784 | 31.599 | 0.721 | 1.00 | 21.72 | C |
| ATOM | 2385 | CG | PHE | B | 53 | 23.562 | 30.113 | 0.699 | 1.00 | 22.04 | C |
| ATOM | 2386 | CD1 | PHE | B | 53 | 22.280 | 29.586 | 0.630 | 1.00 | 23.02 | C |
| ATOM | 2388 | CE1 | PHE | B | 53 | 22.075 | 28.210 | 0.579 | 1.00 | 22.89 | C |
| ATOM | 2390 | CZ | PHE | B | 53 | 23.164 | 27.353 | 0.570 | 1.00 | 22.66 | C |
| ATOM | 2392 | CE2 | PHE | B | 53 | 24.449 | 27.874 | 0.617 | 1.00 | 22.65 | C |
| ATOM | 2394 | CD2 | PHE | B | 53 | 24.639 | 29.243 | 0.670 | 1.00 | 22.77 | C |
| ATOM | 2396 | C | PHE | B | 53 | 25.044 | 33.063 | 2.300 | 1.00 | 21.61 | C |
| ATOM | 2397 | O | PHE | B | 53 | 25.188 | 34.086 | 1.640 | 1.00 | 22.23 | O |
| ATOM | 2399 | N | ILE | B | 54 | 25.955 | 32.649 | 3.178 | 1.00 | 20.80 | N |
| ATOM | 2400 | CA | ILE | B | 54 | 27.197 | 33.392 | 3.390 | 1.00 | 19.84 | C |
| ATOM | 2402 | CB | ILE | B | 54 | 27.345 | 33.888 | 4.855 | 1.00 | 19.39 | C |
| ATOM | 2404 | CG1 | ILE | B | 54 | 27.384 | 32.706 | 5.829 | 1.00 | 20.93 | C |
| ATOM | 2407 | CD1 | ILE | B | 54 | 27.886 | 33.070 | 7.209 | 1.00 | 19.30 | C |
| ATOM | 2411 | CG2 | ILE | B | 54 | 26.218 | 34.858 | 5.218 | 1.00 | 21.15 | C |
| ATOM | 2415 | C | ILE | B | 54 | 28.416 | 32.544 | 3.038 | 1.00 | 19.21 | C |
| ATOM | 2416 | O | ILE | B | 54 | 28.371 | 31.308 | 3.101 | 1.00 | 18.40 | O |
| ATOM | 2418 | N | LYS | B | 55 | 29.502 | 33.222 | 2.685 | 1.00 | 17.72 | N |
| ATOM | 2419 | CA | LYS | B | 55 | 30.780 | 32.563 | 2.518 | 1.00 | 18.97 | C |
| ATOM | 2421 | CB | LYS | B | 55 | 31.661 | 33.265 | 1.482 | 1.00 | 19.33 | C |
| ATOM | 2428 | C | LYS | B | 55 | 31.511 | 32.501 | 3.853 | 1.00 | 19.09 | C |
| ATOM | 2429 | O | LYS | B | 55 | 31.604 | 33.479 | 4.594 | 1.00 | 18.47 | O |
| ATOM | 2431 | N | VAL | B | 56 | 32.069 | 31.335 | 4.107 | 1.00 | 20.73 | N |
| ATOM | 2432 | CA | VAL | B | 56 | 32.852 | 31.092 | 5.303 | 1.00 | 21.27 | C |
| ATOM | 2434 | CB | VAL | B | 56 | 32.052 | 30.247 | 6.309 | 1.00 | 21.79 | C |
| ATOM | 2436 | CG1 | VAL | B | 56 | 30.884 | 31.051 | 6.888 | 1.00 | 21.14 | C |
| ATOM | 2440 | CG2 | VAL | B | 56 | 31.552 | 28.967 | 5.655 | 1.00 | 21.90 | C |
| ATOM | 2444 | C | VAL | B | 56 | 34.136 | 30.383 | 4.911 | 1.00 | 22.55 | C |
| ATOM | 2445 | O | VAL | B | 56 | 34.291 | 29.906 | 3.778 | 1.00 | 22.41 | O |
| ATOM | 2447 | N | ARG | B | 57 | 35.065 | 30.314 | 5.857 | 1.00 | 23.55 | N |
| ATOM | 2448 | CA | ARG | B | 57 | 36.279 | 29.539 | 5.682 | 1.00 | 24.34 | C |
| ATOM | 2450 | CB | ARG | B | 57 | 37.507 | 30.375 | 5.993 | 1.00 | 24.11 | C |
| ATOM | 2453 | CG | ARG | B | 57 | 37.647 | 31.586 | 5.089 | 1.00 | 26.70 | C |
| ATOM | 2456 | CD | ARG | B | 57 | 39.034 | 32.187 | 5.197 | 1.00 | 26.20 | C |
| ATOM | 2459 | NE | ARG | B | 57 | 39.433 | 32.371 | 6.586 | 1.00 | 30.00 | N |
| ATOM | 2461 | CZ | ARG | B | 57 | 39.298 | 33.507 | 7.270 | 1.00 | 32.35 | C |
| ATOM | 2462 | NH1 | ARG | B | 57 | 38.776 | 34.582 | 6.699 | 1.00 | 32.78 | N |
| ATOM | 2465 | NH2 | ARG | B | 57 | 39.687 | 33.574 | 8.537 | 1.00 | 32.60 | N |
| ATOM | 2468 | C | ARG | B | 57 | 36.197 | 28.381 | 6.649 | 1.00 | 24.82 | C |
| ATOM | 2469 | O | ARG | B | 57 | 35.881 | 28.579 | 7.830 | 1.00 | 23.58 | O |
| ATOM | 2471 | N | GLN | B | 58 | 36.425 | 27.178 | 6.123 | 1.00 | 25.04 | N |
| ATOM | 2472 | CA | GLN | B | 58 | 36.275 | 25.948 | 6.895 | 1.00 | 25.92 | C |
| ATOM | 2474 | CB | GLN | B | 58 | 35.577 | 24.858 | 6.069 | 1.00 | 25.42 | C |
| ATOM | 2477 | CG | GLN | B | 58 | 35.487 | 23.515 | 6.789 | 1.00 | 26.50 | C |
| ATOM | 2480 | CD | GLN | B | 58 | 34.679 | 22.482 | 6.029 | 1.00 | 27.48 | C |
| ATOM | 2481 | OE1 | GLN | B | 58 | 33.886 | 22.824 | 5.159 | 1.00 | 30.48 | O |
| ATOM | 2482 | NE2 | GLN | B | 58 | 34.917 | 21.204 | 6.320 | 1.00 | 27.91 | N |
| ATOM | 2485 | C | GLN | B | 58 | 37.633 | 25.452 | 7.363 | 1.00 | 25.42 | C |
| ATOM | 2486 | O | GLN | B | 58 | 38.509 | 25.164 | 6.549 | 1.00 | 25.36 | O |
| ATOM | 2488 | N | TYR | B | 59 | 37.791 | 25.369 | 8.679 | 1.00 | 26.03 | N |
| ATOM | 2489 | CA | TYR | B | 59 | 38.994 | 24.844 | 9.300 | 1.00 | 26.70 | C |
| ATOM | 2491 | CB | TYR | B | 59 | 39.522 | 25.840 | 10.329 | 1.00 | 28.22 | C |
| ATOM | 2494 | CG | TYR | B | 59 | 40.050 | 27.133 | 9.748 | 1.00 | 28.43 | C |
| ATOM | 2495 | CD1 | TYR | B | 59 | 39.213 | 28.216 | 9.519 | 1.00 | 28.69 | C |
| ATOM | 2497 | CE1 | TYR | B | 59 | 39.698 | 29.395 | 8.971 | 1.00 | 27.51 | C |
| ATOM | 2499 | CZ | TYR | B | 59 | 41.041 | 29.514 | 8.701 | 1.00 | 30.03 | C |
| ATOM | 2500 | OH | TYR | B | 59 | 41.575 | 30.677 | 8.184 | 1.00 | 31.05 | O |
| ATOM | 2502 | CE2 | TYR | B | 59 | 41.887 | 28.456 | 8.941 | 1.00 | 31.02 | C |
| ATOM | 2504 | CD2 | TYR | B | 59 | 41.391 | 27.283 | 9.464 | 1.00 | 29.87 | C |
| ATOM | 2506 | C | TYR | B | 59 | 38.663 | 23.518 | 9.975 | 1.00 | 26.65 | C |
| ATOM | 2507 | O | TYR | B | 59 | 37.762 | 23.436 | 10.815 | 1.00 | 27.11 | O |
| ATOM | 2509 | N | ASP | B | 60 | 39.388 | 22.470 | 9.619 | 1.00 | 25.99 | N |
| ATOM | 2510 | CA | ASP | B | 60 | 39.101 | 21.150 | 10.176 | 1.00 | 26.15 | C |
| ATOM | 2512 | CB | ASP | B | 60 | 39.172 | 20.099 | 9.069 | 1.00 | 26.32 | C |
| ATOM | 2515 | CG | ASP | B | 60 | 38.035 | 20.232 | 8.069 | 1.00 | 27.66 | C |
| ATOM | 2516 | OD1 | ASP | B | 60 | 36.901 | 20.539 | 8.499 | 1.00 | 31.18 | O |
| ATOM | 2517 | OD2 | ASP | B | 60 | 38.266 | 20.047 | 6.853 | 1.00 | 28.05 | O |
| ATOM | 2518 | C | ASP | B | 60 | 40.038 | 20.820 | 11.343 | 1.00 | 25.59 | C |
| ATOM | 2519 | O | ASP | B | 60 | 41.151 | 21.329 | 11.411 | 1.00 | 25.79 | O |
| ATOM | 2521 | N | GLN | B | 61 | 39.568 | 20.006 | 12.285 | 1.00 | 25.11 | N |
| ATOM | 2522 | CA | GLN | B | 61 | 40.425 | 19.431 | 13.324 | 1.00 | 25.17 | C |
| ATOM | 2524 | CB | GLN | B | 61 | 41.471 | 18.503 | 12.699 | 1.00 | 25.57 | C |
| ATOM | 2527 | CG | GLN | B | 61 | 40.868 | 17.263 | 12.089 | 1.00 | 28.76 | C |
| ATOM | 2530 | CD | GLN | B | 61 | 40.107 | 16.442 | 13.114 | 1.00 | 33.04 | C |
| ATOM | 2531 | OE1 | GLN | B | 61 | 40.509 | 16.368 | 14.274 | 1.00 | 35.63 | O |
| ATOM | 2532 | NE2 | GLN | B | 61 | 38.997 | 15.834 | 12.696 | 1.00 | 36.23 | N |
| ATOM | 2535 | C | GLN | B | 61 | 41.089 | 20.489 | 14.212 | 1.00 | 24.97 | C |
| ATOM | 2536 | O | GLN | B | 61 | 42.273 | 20.420 | 14.526 | 1.00 | 23.85 | O |
| ATOM | 2538 | N | ILE | B | 62 | 40.279 | 21.447 | 14.639 | 1.00 | 24.81 | N |
| ATOM | 2539 | CA | ILE | B | 62 | 40.727 | 22.570 | 15.440 | 1.00 | 25.66 | C |
| ATOM | 2541 | CB | ILE | B | 62 | 40.018 | 23.863 | 14.989 | 1.00 | 25.45 | C |
| ATOM | 2543 | CG1 | ILE | B | 62 | 40.382 | 24.193 | 13.540 | 1.00 | 26.97 | C |
| ATOM | 2546 | CD1 | ILE | B | 62 | 41.840 | 24.602 | 13.321 | 1.00 | 27.30 | C |
| ATOM | 2550 | CG2 | ILE | B | 62 | 40.360 | 25.008 | 15.903 | 1.00 | 26.92 | C |
| ATOM | 2554 | C | ILE | B | 62 | 40.399 | 22.301 | 16.911 | 1.00 | 25.52 | C |
| ATOM | 2555 | O | ILE | B | 62 | 39.254 | 22.014 | 17.249 | 1.00 | 25.40 | O |
| ATOM | 2557 | N | PRO | B | 63 | 41.414 | 22.363 | 17.786 | 1.00 | 25.71 | N |
| ATOM | 2558 | CA | PRO | B | 63 | 41.111 | 22.227 | 19.200 | 1.00 | 25.15 | C |
| ATOM | 2560 | CB | PRO | B | 63 | 42.486 | 22.115 | 19.873 | 1.00 | 24.92 | C |
| ATOM | 2563 | CG | PRO | B | 63 | 43.481 | 22.038 | 18.794 | 1.00 | 26.82 | C |
| ATOM | 2566 | CD | PRO | B | 63 | 42.854 | 22.537 | 17.535 | 1.00 | 25.27 | C |
| ATOM | 2569 | C | PRO | B | 63 | 40.394 | 23.458 | 19.732 | 1.00 | 25.69 | C |
| ATOM | 2570 | O | PRO | B | 63 | 40.845 | 24.590 | 19.510 | 1.00 | 22.98 | O |
| ATOM | 2571 | N | ILE | B | 64 | 39.323 | 23.217 | 20.476 | 1.00 | 26.08 | N |
| ATOM | 2572 | CA | ILE | B | 64 | 38.607 | 24.289 | 21.138 | 1.00 | 27.90 | C |
| ATOM | 2574 | CB | ILE | B | 64 | 37.450 | 24.784 | 20.267 | 1.00 | 27.54 | C |
| ATOM | 2576 | CG1 | ILE | B | 64 | 36.697 | 25.929 | 20.936 | 1.00 | 28.95 | C |
| ATOM | 2579 | CD1 | ILE | B | 64 | 35.620 | 26.473 | 20.012 | 1.00 | 31.14 | C |
| ATOM | 2583 | CG2 | ILE | B | 64 | 36.500 | 23.680 | 19.952 | 1.00 | 31.86 | C |
| ATOM | 2587 | C | ILE | B | 64 | 38.130 | 23.815 | 22.505 | 1.00 | 27.94 | C |
| ATOM | 2588 | O | ILE | B | 64 | 37.689 | 22.677 | 22.661 | 1.00 | 28.28 | O |
| ATOM | 2590 | N | GLU | B | 65 | 38.333 | 24.663 | 23.509 | 1.00 | 27.23 | N |
| ATOM | 2591 | CA | GLU | B | 65 | 37.942 | 24.352 | 24.859 | 1.00 | 27.95 | C |
| ATOM | 2593 | CB | GLU | B | 65 | 39.083 | 24.673 | 25.814 | 1.00 | 27.83 | C |
| ATOM | 2596 | CG | GLU | B | 65 | 38.791 | 24.314 | 27.251 | 1.00 | 30.44 | C |
| ATOM | 2599 | CD | GLU | B | 65 | 39.919 | 24.721 | 28.172 | 1.00 | 30.29 | C |
| ATOM | 2600 | OE1 | GLU | B | 65 | 41.040 | 24.215 | 27.960 | 1.00 | 31.65 | O |
| ATOM | 2601 | OE2 | GLU | B | 65 | 39.685 | 25.559 | 29.077 | 1.00 | 36.00 | O |
| ATOM | 2602 | C | GLU | B | 65 | 36.708 | 25.169 | 25.212 | 1.00 | 27.00 | C |
| ATOM | 2603 | O | GLU | B | 65 | 36.745 | 26.397 | 25.194 | 1.00 | 26.15 | O |
| ATOM | 2605 | N | ILE | B | 66 | 35.621 | 24.473 | 25.527 | 1.00 | 26.22 | N |
| ATOM | 2606 | CA | ILE | B | 66 | 34.322 | 25.098 | 25.707 | 1.00 | 25.86 | C |
| ATOM | 2608 | CB | ILE | B | 66 | 33.274 | 24.443 | 24.768 | 1.00 | 25.34 | C |
| ATOM | 2610 | CG1 | ILE | B | 66 | 33.743 | 24.530 | 23.311 | 1.00 | 26.31 | C |
| ATOM | 2613 | CD1 | ILE | B | 66 | 33.115 | 23.506 | 22.396 | 1.00 | 26.62 | C |
| ATOM | 2617 | CG2 | ILE | B | 66 | 31.924 | 25.093 | 24.945 | 1.00 | 26.12 | C |
| ATOM | 2621 | C | ILE | B | 66 | 33.907 | 24.968 | 27.164 | 1.00 | 25.71 | C |
| ATOM | 2622 | O | ILE | B | 66 | 33.596 | 23.877 | 27.632 | 1.00 | 24.58 | O |
| ATOM | 2624 | N | CYS | B | 67 | 33.979 | 26.070 | 27.903 | 1.00 | 26.96 | N |
| ATOM | 2625 | CA | CYS | B | 67 | 33.583 | 26.052 | 29.296 | 1.00 | 27.47 | C |
| ATOM | 2627 | CB | CYS | B | 67 | 32.077 | 25.763 | 29.358 | 1.00 | 27.72 | C |
| ATOM | 2630 | SG | CYS | B | 67 | 31.214 | 26.497 | 30.707 | 1.00 | 31.34 | S |
| ATOM | 2632 | C | CYS | B | 67 | 34.394 | 24.980 | 30.041 | 1.00 | 27.36 | C |
| ATOM | 2633 | O | CYS | B | 67 | 33.856 | 24.237 | 30.865 | 1.00 | 27.49 | O |
| ATOM | 2635 | N | GLY | B | 68 | 35.677 | 24.851 | 29.700 | 1.00 | 26.97 | N |
| ATOM | 2636 | CA | GLY | B | 68 | 36.537 | 23.837 | 30.325 | 1.00 | 27.16 | C |
| ATOM | 2639 | C | GLY | B | 68 | 36.546 | 22.451 | 29.688 | 1.00 | 27.48 | C |
| ATOM | 2640 | O | GLY | B | 68 | 37.407 | 21.630 | 30.011 | 1.00 | 27.86 | O |
| ATOM | 2642 | N | HIS | B | 69 | 35.628 | 22.200 | 28.757 | 1.00 | 27.55 | N |
| ATOM | 2643 | CA | HIS | B | 69 | 35.537 | 20.905 | 28.074 | 1.00 | 27.59 | C |
| ATOM | 2645 | CB | HIS | B | 69 | 34.072 | 20.504 | 27.941 | 1.00 | 27.62 | C |
| ATOM | 2648 | CG | HIS | B | 69 | 33.432 | 20.127 | 29.238 | 1.00 | 25.27 | C |
| ATOM | 2649 | ND1 | HIS | B | 69 | 33.089 | 18.830 | 29.546 | 1.00 | 24.65 | N |
| ATOM | 2651 | CE1 | HIS | B | 69 | 32.573 | 18.790 | 30.763 | 1.00 | 25.16 | C |
| ATOM | 2653 | NE2 | HIS | B | 69 | 32.583 | 20.013 | 31.262 | 1.00 | 24.41 | N |
| ATOM | 2655 | CD2 | HIS | B | 69 | 33.108 | 20.870 | 30.324 | 1.00 | 28.01 | C |
| ATOM | 2657 | C | HIS | B | 69 | 36.207 | 20.909 | 26.695 | 1.00 | 27.94 | C |
| ATOM | 2658 | O | HIS | B | 69 | 35.886 | 21.738 | 25.848 | 1.00 | 29.53 | O |
| ATOM | 2660 | N | LYS | B | 70 | 37.113 | 19.962 | 26.455 | 1.00 | 27.88 | N |
| ATOM | 2661 | CA | LYS | B | 70 | 37.903 | 19.933 | 25.218 | 1.00 | 27.95 | C |
| ATOM | 2663 | CB | LYS | B | 70 | 39.212 | 19.167 | 25.432 | 1.00 | 28.28 | C |
| ATOM | 2666 | CG | LYS | B | 70 | 40.237 | 19.881 | 26.303 | 1.00 | 28.86 | C |
| ATOM | 2669 | CD | LYS | B | 70 | 41.441 | 18.975 | 26.589 | 1.00 | 29.11 | C |
| ATOM | 2672 | CE | LYS | B | 70 | 42.534 | 19.699 | 27.354 | 1.00 | 30.19 | C |
| ATOM | 2675 | NZ | LYS | B | 70 | 43.368 | 18.766 | 28.185 | 1.00 | 32.28 | N |
| ATOM | 2679 | C | LYS | B | 70 | 37.165 | 19.308 | 24.030 | 1.00 | 28.37 | C |
| ATOM | 2680 | O | LYS | B | 70 | 36.628 | 18.201 | 24.123 | 1.00 | 28.38 | O |
| ATOM | 2682 | N | ALA | B | 71 | 37.174 | 20.018 | 22.899 | 1.00 | 28.07 | N |
| ATOM | 2683 | CA | ALA | B | 71 | 36.751 | 19.470 | 21.613 | 1.00 | 27.27 | C |
| ATOM | 2685 | CB | ALA | B | 71 | 35.443 | 20.135 | 21.157 | 1.00 | 27.81 | C |
| ATOM | 2689 | C | ALA | B | 71 | 37.843 | 19.669 | 20.562 | 1.00 | 27.26 | C |
| ATOM | 2690 | O | ALA | B | 71 | 38.677 | 20.574 | 20.671 | 1.00 | 25.37 | O |
| ATOM | 2692 | N | ILE | B | 72 | 37.828 | 18.813 | 19.543 | 1.00 | 26.82 | N |
| ATOM | 2693 | CA | ILE | B | 72 | 38.668 | 18.993 | 18.355 | 1.00 | 26.50 | C |
| ATOM | 2695 | CB | ILE | B | 72 | 39.864 | 18.035 | 18.317 | 1.00 | 26.21 | C |
| ATOM | 2697 | CG1 | ILE | B | 72 | 40.633 | 18.051 | 19.639 | 1.00 | 25.71 | C |
| ATOM | 2700 | CD1 | ILE | B | 72 | 41.610 | 16.908 | 19.792 | 1.00 | 27.08 | C |
| ATOM | 2704 | CG2 | ILE | B | 72 | 40.776 | 18.381 | 17.138 | 1.00 | 25.53 | C |
| ATOM | 2708 | C | ILE | B | 72 | 37.798 | 18.707 | 17.134 | 1.00 | 26.71 | C |
| ATOM | 2709 | O | ILE | B | 72 | 37.361 | 17.576 | 16.935 | 1.00 | 27.16 | O |
| ATOM | 2711 | N | GLY | B | 73 | 37.531 | 19.735 | 16.336 | 1.00 | 25.89 | N |
| ATOM | 2712 | CA | GLY | B | 73 | 36.516 | 19.617 | 15.314 | 1.00 | 26.68 | C |
| ATOM | 2715 | C | GLY | B | 73 | 36.519 | 20.742 | 14.304 | 1.00 | 26.13 | C |
| ATOM | 2716 | O | GLY | B | 73 | 37.421 | 21.569 | 14.278 | 1.00 | 25.69 | O |
| ATOM | 2718 | N | THR | B | 74 | 35.495 | 20.749 | 13.463 | 1.00 | 27.28 | N |
| ATOM | 2719 | CA | THR | B | 74 | 35.419 | 21.675 | 12.328 | 1.00 | 28.23 | C |
| ATOM | 2721 | CB | THR | B | 74 | 34.540 | 21.079 | 11.212 | 1.00 | 28.57 | C |
| ATOM | 2723 | OG1 | THR | B | 74 | 35.171 | 19.891 | 10.715 | 1.00 | 28.73 | O |
| ATOM | 2725 | CG2 | THR | B | 74 | 34.332 | 22.065 | 10.043 | 1.00 | 28.38 | C |
| ATOM | 2729 | C | THR | B | 74 | 34.875 | 23.011 | 12.822 | 1.00 | 28.11 | C |
| ATOM | 2730 | O | THR | B | 74 | 33.878 | 23.051 | 13.536 | 1.00 | 27.25 | O |
| ATOM | 2732 | N | VAL | B | 75 | 35.584 | 24.088 | 12.494 | 1.00 | 28.17 | N |
| ATOM | 2733 | CA | VAL | B | 75 | 35.169 | 25.439 | 12.823 | 1.00 | 28.56 | C |
| ATOM | 2735 | CB | VAL | B | 75 | 36.199 | 26.111 | 13.764 | 1.00 | 28.96 | C |
| ATOM | 2737 | CG1 | VAL | B | 75 | 35.982 | 27.599 | 13.818 | 1.00 | 31.83 | C |
| ATOM | 2741 | CG2 | VAL | B | 75 | 36.135 | 25.509 | 15.161 | 1.00 | 29.84 | C |
| ATOM | 2745 | C | VAL | B | 75 | 35.038 | 26.250 | 11.526 | 1.00 | 28.12 | C |
| ATOM | 2746 | O | VAL | B | 75 | 35.963 | 26.291 | 10.705 | 1.00 | 28.89 | O |
| ATOM | 2748 | N | LEU | B | 76 | 33.873 | 26.861 | 11.332 | 1.00 | 27.01 | N |
| ATOM | 2749 | CA | LEU | B | 76 | 33.651 | 27.771 | 10.222 | 1.00 | 26.32 | C |
| ATOM | 2751 | CB | LEU | B | 76 | 32.231 | 27.603 | 9.668 | 1.00 | 26.00 | C |
| ATOM | 2754 | CG | LEU | B | 76 | 31.850 | 26.163 | 9.332 | 1.00 | 26.37 | C |
| ATOM | 2756 | CD1 | LEU | B | 76 | 30.388 | 26.066 | 8.917 | 1.00 | 24.76 | C |
| ATOM | 2760 | CD2 | LEU | B | 76 | 32.809 | 25.600 | 8.247 | 1.00 | 26.83 | C |
| ATOM | 2764 | C | LEU | B | 76 | 33.878 | 29.213 | 10.702 | 1.00 | 26.04 | C |
| ATOM | 2765 | O | LEU | B | 76 | 33.404 | 29.596 | 11.775 | 1.00 | 27.41 | O |
| ATOM | 2767 | N | VAL | B | 77 | 34.657 | 29.974 | 9.941 | 1.00 | 24.11 | N |
| ATOM | 2768 | CA | VAL | B | 77 | 34.943 | 31.381 | 10.254 | 1.00 | 23.31 | C |
| ATOM | 2770 | CB | VAL | B | 77 | 36.471 | 31.635 | 10.366 | 1.00 | 23.53 | C |
| ATOM | 2772 | CG1 | VAL | B | 77 | 36.773 | 33.099 | 10.663 | 1.00 | 23.57 | C |
| ATOM | 2776 | CG2 | VAL | B | 77 | 37.067 | 30.772 | 11.446 | 1.00 | 23.50 | C |
| ATOM | 2780 | C | VAL | B | 77 | 34.316 | 32.272 | 9.174 | 1.00 | 24.07 | C |
| ATOM | 2781 | O | VAL | B | 77 | 34.517 | 32.066 | 7.976 | 1.00 | 22.45 | O |
| ATOM | 2783 | N | GLY | B | 78 | 33.490 | 33.213 | 9.616 | 1.00 | 24.33 | N |
| ATOM | 2784 | CA | GLY | B | 78 | 32.848 | 34.144 | 8.725 | 1.00 | 24.88 | C |
| ATOM | 2787 | C | GLY | B | 78 | 31.946 | 35.111 | 9.463 | 1.00 | 25.40 | C |
| ATOM | 2788 | O | GLY | B | 78 | 31.894 | 35.109 | 10.704 | 1.00 | 24.67 | O |
| ATOM | 2790 | N | PRO | B | 79 | 31.203 | 35.923 | 8.697 | 1.00 | 26.37 | N |
| ATOM | 2791 | CA | PRO | B | 79 | 30.348 | 37.004 | 9.207 | 1.00 | 27.09 | C |
| ATOM | 2793 | CB | PRO | B | 79 | 29.999 | 37.786 | 7.939 | 1.00 | 27.10 | C |
| ATOM | 2796 | CG | PRO | B | 79 | 30.023 | 36.745 | 6.854 | 1.00 | 26.69 | C |
| ATOM | 2799 | CD | PRO | B | 79 | 31.116 | 35.797 | 7.230 | 1.00 | 26.39 | C |
| ATOM | 2802 | C | PRO | B | 79 | 29.072 | 36.513 | 9.902 | 1.00 | 27.82 | C |
| ATOM | 2803 | O | PRO | B | 79 | 28.031 | 36.397 | 9.280 | 1.00 | 29.09 | O |
| ATOM | 2804 | N | THR | B | 80 | 29.138 | 36.274 | 11.203 | 1.00 | 29.11 | N |
| ATOM | 2805 | CA | THR | B | 80 | 27.974 | 35.780 | 11.931 | 1.00 | 28.76 | C |
| ATOM | 2807 | CB | THR | B | 80 | 28.286 | 34.417 | 12.591 | 1.00 | 28.72 | C |
| ATOM | 2809 | OG1 | THR | B | 80 | 27.185 | 33.981 | 13.407 | 1.00 | 27.11 | O |
| ATOM | 2811 | CG2 | THR | B | 80 | 29.583 | 34.489 | 13.412 | 1.00 | 29.65 | C |
| ATOM | 2815 | C | THR | B | 80 | 27.546 | 36.848 | 12.932 | 1.00 | 29.30 | C |
| ATOM | 2816 | O | THR | B | 80 | 28.387 | 37.612 | 13.409 | 1.00 | 29.19 | O |
| ATOM | 2818 | N | PRO | B | 81 | 26.236 | 36.934 | 13.242 | 1.00 | 29.17 | N |
| ATOM | 2819 | CA | PRO | B | 81 | 25.846 | 37.895 | 14.279 | 1.00 | 29.05 | C |
| ATOM | 2821 | CB | PRO | B | 81 | 24.334 | 37.694 | 14.411 | 1.00 | 28.63 | C |
| ATOM | 2824 | CG | PRO | B | 81 | 23.921 | 36.944 | 13.202 | 1.00 | 29.03 | C |
| ATOM | 2827 | CD | PRO | B | 81 | 25.086 | 36.163 | 12.733 | 1.00 | 29.48 | C |
| ATOM | 2830 | C | PRO | B | 81 | 26.526 | 37.640 | 15.630 | 1.00 | 28.60 | C |
| ATOM | 2831 | O | PRO | B | 81 | 26.684 | 38.560 | 16.422 | 1.00 | 27.43 | O |
| ATOM | 2832 | N | VAL | B | 82 | 26.886 | 36.385 | 15.890 | 1.00 | 28.53 | N |
| ATOM | 2833 | CA | VAL | B | 82 | 27.429 | 35.967 | 17.177 | 1.00 | 28.05 | C |
| ATOM | 2835 | CB | VAL | B | 82 | 26.295 | 35.701 | 18.206 | 1.00 | 28.72 | C |
| ATOM | 2837 | CG1 | VAL | B | 82 | 25.336 | 34.637 | 17.694 | 1.00 | 29.60 | C |
| ATOM | 2841 | CG2 | VAL | B | 82 | 26.872 | 35.277 | 19.550 | 1.00 | 29.66 | C |
| ATOM | 2845 | C | VAL | B | 82 | 28.218 | 34.677 | 16.976 | 1.00 | 27.20 | C |
| ATOM | 2846 | O | VAL | B | 82 | 27.909 | 33.902 | 16.065 | 1.00 | 26.52 | O |
| ATOM | 2848 | N | ASN | B | 83 | 29.217 | 34.442 | 17.827 | 1.00 | 25.88 | N |
| ATOM | 2849 | CA | ASN | B | 83 | 29.985 | 33.201 | 17.779 | 1.00 | 25.95 | C |
| ATOM | 2851 | CB | ASN | B | 83 | 31.171 | 33.241 | 18.746 | 1.00 | 25.17 | C |
| ATOM | 2854 | CG | ASN | B | 83 | 32.280 | 34.183 | 18.302 | 1.00 | 26.98 | C |
| ATOM | 2855 | OD1 | ASN | B | 83 | 32.622 | 34.254 | 17.114 | 1.00 | 29.10 | O |
| ATOM | 2856 | ND2 | ASN | B | 83 | 32.883 | 34.881 | 19.266 | 1.00 | 22.37 | N |
| ATOM | 2859 | C | ASN | B | 83 | 29.030 | 32.093 | 18.175 | 1.00 | 25.74 | C |
| ATOM | 2860 | O | ASN | B | 83 | 28.306 | 32.243 | 19.150 | 1.00 | 26.26 | O |
| ATOM | 2862 | N | ILE | B | 84 | 29.010 | 31.007 | 17.406 | 1.00 | 27.00 | N |
| ATOM | 2863 | CA | ILE | B | 84 | 28.014 | 29.935 | 17.538 | 1.00 | 26.90 | C |
| ATOM | 2865 | CB | ILE | B | 84 | 27.129 | 29.863 | 16.279 | 1.00 | 27.19 | C |
| ATOM | 2867 | CG1 | ILE | B | 84 | 26.161 | 31.049 | 16.241 | 1.00 | 27.40 | C |
| ATOM | 2870 | CD1 | ILE | B | 84 | 25.613 | 31.328 | 14.859 | 1.00 | 29.05 | C |
| ATOM | 2874 | CG2 | ILE | B | 84 | 26.380 | 28.540 | 16.202 | 1.00 | 27.16 | C |
| ATOM | 2878 | C | ILE | B | 84 | 28.706 | 28.587 | 17.718 | 1.00 | 27.21 | C |
| ATOM | 2879 | O | ILE | B | 84 | 29.450 | 28.140 | 16.844 | 1.00 | 26.20 | O |
| ATOM | 2881 | N | ILE | B | 85 | 28.470 | 27.954 | 18.867 | 1.00 | 26.59 | N |
| ATOM | 2882 | CA | ILE | B | 85 | 28.862 | 26.569 | 19.062 | 1.00 | 26.04 | C |
| ATOM | 2884 | CB | ILE | B | 85 | 29.295 | 26.295 | 20.491 | 1.00 | 25.35 | C |
| ATOM | 2886 | CG1 | ILE | B | 85 | 30.340 | 27.314 | 20.923 | 1.00 | 25.15 | C |
| ATOM | 2889 | CD1 | ILE | B | 85 | 31.593 | 27.339 | 20.019 | 1.00 | 28.04 | C |
| ATOM | 2893 | CG2 | ILE | B | 85 | 29.811 | 24.855 | 20.629 | 1.00 | 28.27 | C |
| ATOM | 2897 | C | ILE | B | 85 | 27.701 | 25.665 | 18.672 | 1.00 | 25.99 | C |
| ATOM | 2898 | O | ILE | B | 85 | 26.674 | 25.600 | 19.356 | 1.00 | 26.09 | O |
| ATOM | 2900 | N | GLY | B | 86 | 27.889 | 24.944 | 17.571 | 1.00 | 26.39 | N |
| ATOM | 2901 | CA | GLY | B | 86 | 26.873 | 24.032 | 17.071 | 1.00 | 26.02 | C |
| ATOM | 2904 | C | GLY | B | 86 | 27.032 | 22.575 | 17.471 | 1.00 | 25.89 | C |
| ATOM | 2905 | O | GLY | B | 86 | 27.939 | 22.201 | 18.217 | 1.00 | 26.68 | O |
| ATOM | 2907 | N | ARG | B | 87 | 26.127 | 21.746 | 16.968 | 1.00 | 25.80 | N |
| ATOM | 2908 | CA | ARG | B | 87 | 26.005 | 20.372 | 17.429 | 1.00 | 26.04 | C |
| ATOM | 2910 | CB | ARG | B | 87 | 24.783 | 19.702 | 16.790 | 1.00 | 26.31 | C |
| ATOM | 2913 | CG | ARG | B | 87 | 23.453 | 20.219 | 17.342 | 1.00 | 26.44 | C |
| ATOM | 2916 | CD | ARG | B | 87 | 22.250 | 19.457 | 16.809 | 1.00 | 26.97 | C |
| ATOM | 2919 | NE | ARG | B | 87 | 22.204 | 19.441 | 15.344 | 1.00 | 28.08 | N |
| ATOM | 2921 | CZ | ARG | B | 87 | 22.582 | 18.410 | 14.587 | 1.00 | 30.48 | C |
| ATOM | 2922 | NH1 | ARG | B | 87 | 23.066 | 17.296 | 15.123 | 1.00 | 28.02 | N |
| ATOM | 2925 | NH2 | ARG | B | 87 | 22.524 | 18.516 | 13.269 | 1.00 | 31.63 | N |
| ATOM | 2928 | C | ARG | B | 87 | 27.270 | 19.541 | 17.202 | 1.00 | 26.14 | C |
| ATOM | 2929 | O | ARG | B | 87 | 27.548 | 18.613 | 17.961 | 1.00 | 26.89 | O |
| ATOM | 2931 | N | ASN | B | 88 | 28.028 | 19.854 | 16.161 | 1.00 | 25.86 | N |
| ATOM | 2932 | CA | ASN | B | 88 | 29.251 | 19.109 | 15.887 | 1.00 | 26.55 | C |
| ATOM | 2934 | CB | ASN | B | 88 | 29.933 | 19.629 | 14.612 | 1.00 | 25.71 | C |
| ATOM | 2937 | CG | ASN | B | 88 | 30.626 | 20.960 | 14.803 | 1.00 | 28.29 | C |
| ATOM | 2938 | OD1 | ASN | B | 88 | 30.035 | 21.919 | 15.312 | 1.00 | 26.22 | O |
| ATOM | 2939 | ND2 | ASN | B | 88 | 31.876 | 21.044 | 14.332 | 1.00 | 26.83 | N |
| ATOM | 2942 | C | ASN | B | 88 | 30.211 | 19.128 | 17.078 | 1.00 | 26.40 | C |
| ATOM | 2943 | O | ASN | B | 88 | 30.930 | 18.154 | 17.300 | 1.00 | 26.90 | O |
| ATOM | 2945 | N | LEU | B | 89 | 30.245 | 20.236 | 17.822 | 1.00 | 26.30 | N |
| ATOM | 2946 | CA | LEU | B | 89 | 31.095 | 20.330 | 19.013 | 1.00 | 26.32 | C |
| ATOM | 2948 | CB | LEU | B | 89 | 31.790 | 21.688 | 19.093 | 1.00 | 26.14 | C |
| ATOM | 2951 | CG | LEU | B | 89 | 32.697 | 22.043 | 17.908 | 1.00 | 25.61 | C |
| ATOM | 2953 | CD1 | LEU | B | 89 | 33.520 | 23.283 | 18.204 | 1.00 | 28.00 | C |
| ATOM | 2957 | CD2 | LEU | B | 89 | 33.588 | 20.893 | 17.507 | 1.00 | 27.40 | C |
| ATOM | 2961 | C | LEU | B | 89 | 30.339 | 20.073 | 20.309 | 1.00 | 26.43 | C |
| ATOM | 2962 | O | LEU | B | 89 | 30.924 | 19.621 | 21.289 | 1.00 | 25.92 | O |
| ATOM | 2964 | N | LEU | B | 90 | 29.055 | 20.409 | 20.327 | 1.00 | 26.62 | N |
| ATOM | 2965 | CA | LEU | B | 90 | 28.227 | 20.166 | 21.508 | 1.00 | 26.51 | C |
| ATOM | 2967 | CB | LEU | B | 90 | 26.832 | 20.718 | 21.293 | 1.00 | 26.55 | C |
| ATOM | 2970 | CG | LEU | B | 90 | 26.707 | 22.235 | 21.240 | 1.00 | 26.72 | C |
| ATOM | 2972 | CD1 | LEU | B | 90 | 25.266 | 22.611 | 21.020 | 1.00 | 27.88 | C |
| ATOM | 2976 | CD2 | LEU | B | 90 | 27.204 | 22.839 | 22.522 | 1.00 | 28.08 | C |
| ATOM | 2980 | C | LEU | B | 90 | 28.157 | 18.686 | 21.881 | 1.00 | 25.96 | C |
| ATOM | 2981 | O | LEU | B | 90 | 28.174 | 18.321 | 23.066 | 1.00 | 25.16 | O |
| ATOM | 2983 | N | THR | B | 91 | 28.109 | 17.845 | 20.851 | 1.00 | 25.78 | N |
| ATOM | 2984 | CA | THR | B | 91 | 28.145 | 16.416 | 21.037 | 1.00 | 25.23 | C |
| ATOM | 2986 | CB | THR | B | 91 | 27.869 | 15.664 | 19.737 | 1.00 | 25.56 | C |
| ATOM | 2988 | OG1 | THR | B | 91 | 28.759 | 16.112 | 18.701 | 1.00 | 25.01 | O |
| ATOM | 2990 | CG2 | THR | B | 91 | 26.426 | 15.871 | 19.342 | 1.00 | 26.81 | C |
| ATOM | 2994 | C | THR | B | 91 | 29.483 | 15.987 | 21.598 | 1.00 | 25.96 | C |
| ATOM | 2995 | O | THR | B | 91 | 29.525 | 15.143 | 22.483 | 1.00 | 25.74 | O |
| ATOM | 2997 | N | GLN | B | 92 | 30.564 | 16.616 | 21.148 | 1.00 | 25.79 | N |
| ATOM | 2998 | CA | GLN | B | 92 | 31.888 | 16.267 | 21.664 | 1.00 | 26.04 | C |
| ATOM | 3000 | CB | GLN | B | 92 | 32.995 | 17.012 | 20.915 | 1.00 | 25.47 | C |
| ATOM | 3003 | CG | GLN | B | 92 | 33.261 | 16.424 | 19.544 | 1.00 | 26.50 | C |
| ATOM | 3006 | CD | GLN | B | 92 | 34.597 | 16.856 | 18.972 | 1.00 | 26.87 | C |
| ATOM | 3007 | OE1 | GLN | B | 92 | 35.535 | 17.163 | 19.719 | 1.00 | 25.48 | O |
| ATOM | 3008 | NE2 | GLN | B | 92 | 34.678 | 16.925 | 17.645 | 1.00 | 25.06 | N |
| ATOM | 3011 | C | GLN | B | 92 | 32.007 | 16.508 | 23.166 | 1.00 | 26.53 | C |
| ATOM | 3012 | O | GLN | B | 92 | 32.626 | 15.709 | 23.867 | 1.00 | 26.04 | O |
| ATOM | 3014 | N | ILE | B | 93 | 31.377 | 17.566 | 23.673 | 1.00 | 25.67 | N |
| ATOM | 3015 | CA | ILE | B | 93 | 31.508 | 17.887 | 25.092 | 1.00 | 26.51 | C |
| ATOM | 3017 | CB | ILE | B | 93 | 31.578 | 19.414 | 25.308 | 1.00 | 26.87 | C |
| ATOM | 3019 | CG1 | ILE | B | 93 | 30.273 | 20.090 | 24.893 | 1.00 | 27.09 | C |
| ATOM | 3022 | CD1 | ILE | B | 93 | 30.248 | 21.575 | 25.210 | 1.00 | 27.45 | C |
| ATOM | 3026 | CG2 | ILE | B | 93 | 32.759 | 20.021 | 24.511 | 1.00 | 26.00 | C |
| ATOM | 3030 | C | ILE | B | 93 | 30.402 | 17.259 | 25.965 | 1.00 | 26.77 | C |
| ATOM | 3031 | O | ILE | B | 93 | 30.297 | 17.554 | 27.157 | 1.00 | 27.75 | O |
| ATOM | 3033 | N | GLY | B | 94 | 29.571 | 16.408 | 25.375 | 1.00 | 26.21 | N |
| ATOM | 3034 | CA | GLY | B | 94 | 28.584 | 15.652 | 26.149 | 1.00 | 26.50 | C |
| ATOM | 3037 | C | GLY | B | 94 | 27.316 | 16.436 | 26.442 | 1.00 | 26.79 | C |
| ATOM | 3038 | O | GLY | B | 94 | 26.573 | 16.125 | 27.378 | 1.00 | 25.37 | O |
| ATOM | 3040 | N | CYS | B | 95 | 27.056 | 17.448 | 25.631 | 1.00 | 27.64 | N |
| ATOM | 3041 | CA | CYS | B | 95 | 25.933 | 18.341 | 25.884 | 1.00 | 28.31 | C |
| ATOM | 3043 | CB | CYS | B | 95 | 26.179 | 19.707 | 25.234 | 1.00 | 28.88 | C |
| ATOM | 3046 | SG | CYS | B | 95 | 24.991 | 20.943 | 25.743 | 1.00 | 32.45 | S |
| ATOM | 3048 | C | CYS | B | 95 | 24.620 | 17.711 | 25.423 | 1.00 | 27.72 | C |
| ATOM | 3049 | O | CYS | B | 95 | 24.555 | 17.117 | 24.350 | 1.00 | 28.58 | O |
| ATOM | 3051 | N | THR | B | 96 | 23.617 | 17.735 | 26.303 | 1.00 | 27.31 | N |
| ATOM | 3052 | CA | THR | B | 96 | 22.267 | 17.308 | 25.978 | 1.00 | 26.13 | C |
| ATOM | 3054 | CB | THR | B | 96 | 21.915 | 15.974 | 26.683 | 1.00 | 26.57 | C |
| ATOM | 3056 | OG1 | THR | B | 96 | 21.952 | 16.160 | 28.103 | 1.00 | 24.28 | O |
| ATOM | 3058 | CG2 | THR | B | 96 | 22.909 | 14.860 | 26.302 | 1.00 | 27.64 | C |
| ATOM | 3062 | C | THR | B | 96 | 21.247 | 18.361 | 26.419 | 1.00 | 26.37 | C |
| ATOM | 3063 | O | THR | B | 96 | 21.534 | 19.239 | 27.240 | 1.00 | 25.66 | O |
| ATOM | 3065 | N | LEU | B | 97 | 20.041 | 18.249 | 25.875 | 1.00 | 26.34 | N |
| ATOM | 3066 | CA | LEU | B | 97 | 18.909 | 19.031 | 26.333 | 1.00 | 26.12 | C |
| ATOM | 3068 | CB | LEU | B | 97 | 18.060 | 19.470 | 25.154 | 1.00 | 26.26 | C |
| ATOM | 3071 | CG | LEU | B | 97 | 18.544 | 20.632 | 24.312 | 1.00 | 27.02 | C |
| ATOM | 3073 | CD1 | LEU | B | 97 | 17.807 | 20.574 | 22.975 | 1.00 | 26.63 | C |
| ATOM | 3077 | CD2 | LEU | B | 97 | 18.251 | 21.951 | 25.057 | 1.00 | 27.89 | C |
| ATOM | 3081 | C | LEU | B | 97 | 18.038 | 18.172 | 27.243 | 1.00 | 26.14 | C |
| ATOM | 3082 | O | LEU | B | 97 | 17.867 | 16.989 | 26.998 | 1.00 | 25.11 | O |
| ATOM | 3084 | N | ASN | B | 98 | 17.498 | 18.781 | 28.287 | 1.00 | 25.93 | N |
| ATOM | 3085 | CA | ASN | B | 98 | 16.704 | 18.066 | 29.273 | 1.00 | 26.90 | C |
| ATOM | 3087 | CB | ASN | B | 98 | 17.570 | 17.667 | 30.470 | 1.00 | 26.87 | C |
| ATOM | 3090 | CG | ASN | B | 98 | 18.810 | 16.867 | 30.060 | 1.00 | 28.22 | C |
| ATOM | 3091 | OD1 | ASN | B | 98 | 18.808 | 15.640 | 30.112 | 1.00 | 31.52 | O |
| ATOM | 3092 | ND2 | ASN | B | 98 | 19.851 | 17.555 | 29.616 | 1.00 | 26.96 | N |
| ATOM | 3095 | C | ASN | B | 98 | 15.536 | 18.930 | 29.726 | 1.00 | 27.18 | C |
| ATOM | 3096 | O | ASN | B | 98 | 15.684 | 20.129 | 29.932 | 1.00 | 25.99 | O |
| ATOM | 3098 | N | PHE | B | 99 | 14.360 | 18.326 | 29.816 | 1.00 | 28.59 | N |
| ATOM | 3099 | CA | PHE | B | 99 | 13.240 | 18.946 | 30.508 | 1.00 | 30.19 | C |
| ATOM | 3101 | CB | PHE | B | 99 | 12.430 | 19.856 | 29.572 | 1.00 | 31.41 | C |
| ATOM | 3104 | CG | PHE | B | 99 | 11.953 | 19.181 | 28.322 | 1.00 | 32.03 | C |
| ATOM | 3105 | CD1 | PHE | B | 99 | 12.776 | 19.088 | 27.212 | 1.00 | 34.97 | C |
| ATOM | 3107 | CE1 | PHE | B | 99 | 12.338 | 18.460 | 26.053 | 1.00 | 35.62 | C |
| ATOM | 3109 | CZ | PHE | B | 99 | 11.064 | 17.932 | 25.993 | 1.00 | 34.47 | C |
| ATOM | 3111 | CE2 | PHE | B | 99 | 10.232 | 18.022 | 27.096 | 1.00 | 35.16 | C |
| ATOM | 3113 | CD2 | PHE | B | 99 | 10.677 | 18.648 | 28.251 | 1.00 | 34.40 | C |
| ATOM | 3115 | C | PHE | B | 99 | 12.391 | 17.827 | 31.075 | 1.00 | 30.39 | C |
| ATOM | 3116 | O | PHE | B | 99 | 12.870 | 16.697 | 31.130 | 1.00 | 30.70 | O |
| ATOM | 3118 | OXT | PHE | B | 99 | 11.254 | 18.022 | 31.491 | 1.00 | 30.57 | O |
| TER | | | | | | | | | | | |
| HETATM | 3119 | O9 | MUT | C | 200 | 19.834 | 30.807 | 10.483 | 1.00 | 28.31 | O |
| HETATM | 3120 | S7 | MUT | C | 200 | 20.554 | 30.591 | 11.697 | 1.00 | 27.32 | S |
| HETATM | 3121 | O8 | MUT | C | 200 | 21.059 | 31.839 | 12.187 | 1.00 | 25.87 | O |
| HETATM | 3122 | C1 | MUT | C | 200 | 21.783 | 29.573 | 11.421 | 1.00 | 28.01 | C |
| HETATM | 3123 | C2 | MUT | C | 200 | 21.929 | 28.855 | 10.228 | 1.00 | 28.62 | C |
| HETATM | 3125 | C3 | MUT | C | 200 | 23.005 | 27.976 | 10.034 | 1.00 | 28.35 | C |
| HETATM | 3127 | C4 | MUT | C | 200 | 23.955 | 27.781 | 11.041 | 1.00 | 27.26 | C |
| HETATM | 3128 | C15 | MUT | C | 200 | 25.163 | 26.839 | 10.920 | 1.00 | 27.32 | C |
| HETATM | 3131 | N45 | MUT | C | 200 | 25.131 | 25.999 | 9.734 | 1.00 | 24.63 | N |
| HETATM | 3134 | C5 | MUT | C | 200 | 23.814 | 28.501 | 12.218 | 1.00 | 26.55 | C |
| HETATM | 3136 | C6 | MUT | C | 200 | 22.755 | 29.382 | 12.400 | 1.00 | 29.13 | C |
| HETATM | 3138 | N10 | MUT | C | 200 | 19.474 | 29.961 | 12.728 | 1.00 | 29.20 | N |
| HETATM | 3139 | C11 | MUT | C | 200 | 19.211 | 28.574 | 12.315 | 1.00 | 32.65 | C |
| HETATM | 3142 | C12 | MUT | C | 200 | 17.739 | 28.211 | 12.432 | 1.00 | 33.47 | C |
| HETATM | 3144 | C13 | MUT | C | 200 | 17.549 | 26.786 | 11.927 | 1.00 | 33.15 | C |
| HETATM | 3148 | C14 | MUT | C | 200 | 16.889 | 29.144 | 11.580 | 1.00 | 35.04 | C |
| HETATM | 3152 | C16 | MUT | C | 200 | 19.846 | 30.187 | 14.133 | 1.00 | 29.57 | C |
| HETATM | 3154 | C22 | MUT | C | 200 | 19.929 | 28.925 | 14.986 | 1.00 | 27.39 | C |
| HETATM | 3157 | O23 | MUT | C | 200 | 20.041 | 29.278 | 16.391 | 1.00 | 27.88 | O |
| HETATM | 3159 | C17 | MUT | C | 200 | 18.937 | 31.202 | 14.844 | 1.00 | 31.10 | C |
| HETATM | 3162 | C18 | MUT | C | 200 | 17.624 | 31.518 | 14.165 | 1.00 | 32.95 | C |
| HETATM | 3165 | C19 | MUT | C | 200 | 17.300 | 33.015 | 14.152 | 1.00 | 30.51 | C |
| HETATM | 3168 | C20 | MUT | C | 200 | 16.899 | 33.600 | 15.504 | 1.00 | 28.91 | C |
| HETATM | 3171 | N21 | MUT | C | 200 | 17.784 | 34.731 | 15.771 | 1.00 | 27.24 | N |
| HETATM | 3172 | C39 | MUT | C | 200 | 18.010 | 35.213 | 16.994 | 1.00 | 27.74 | C |
| HETATM | 3173 | O40 | MUT | C | 200 | 17.513 | 34.806 | 18.037 | 1.00 | 29.38 | O |
| HETATM | 3174 | C37 | MUT | C | 200 | 19.010 | 36.351 | 17.060 | 1.00 | 26.24 | C |
| HETATM | 3176 | N38 | MUT | C | 200 | 18.479 | 37.361 | 17.952 | 1.00 | 26.50 | N |
| HETATM | 3177 | C41 | MUT | C | 200 | 18.133 | 38.548 | 17.476 | 1.00 | 30.17 | C |
| HETATM | 3178 | O42 | MUT | C | 200 | 17.585 | 39.543 | 18.380 | 1.00 | 31.13 | O |
| HETATM | 3179 | C44 | MUT | C | 200 | 16.624 | 40.487 | 17.912 | 1.00 | 30.43 | C |
| HETATM | 3183 | O43 | MUT | C | 200 | 18.282 | 38.806 | 16.290 | 1.00 | 30.93 | O |
| HETATM | 3184 | C30 | MUT | C | 200 | 20.291 | 35.813 | 17.694 | 1.00 | 26.22 | C |
| HETATM | 3186 | C24 | MUT | C | 200 | 21.343 | 36.897 | 17.932 | 1.00 | 27.92 | C |
| HETATM | 3187 | C25 | MUT | C | 200 | 21.506 | 37.966 | 17.048 | 1.00 | 27.58 | C |
| HETATM | 3189 | C26 | MUT | C | 200 | 22.501 | 38.909 | 17.263 | 1.00 | 27.17 | C |
| HETATM | 3191 | C27 | MUT | C | 200 | 23.324 | 38.790 | 18.377 | 1.00 | 28.09 | C |
| HETATM | 3193 | C28 | MUT | C | 200 | 23.169 | 37.722 | 19.257 | 1.00 | 27.94 | C |
| HETATM | 3195 | C29 | MUT | C | 200 | 22.189 | 36.769 | 19.032 | 1.00 | 27.50 | C |
| HETATM | 3197 | C31 | MUT | C | 200 | 20.958 | 34.757 | 16.817 | 1.00 | 25.27 | C |
| HETATM | 3198 | C32 | MUT | C | 200 | 21.060 | 33.455 | 17.284 | 1.00 | 26.40 | C |
| HETATM | 3200 | C33 | MUT | C | 200 | 21.726 | 32.489 | 16.541 | 1.00 | 25.49 | C |
| HETATM | 3202 | C34 | MUT | C | 200 | 22.275 | 32.827 | 15.315 | 1.00 | 24.21 | C |
| HETATM | 3204 | C35 | MUT | C | 200 | 22.175 | 34.123 | 14.838 | 1.00 | 23.62 | C |
| HETATM | 3206 | C36 | MUT | C | 200 | 21.530 | 35.089 | 15.593 | 1.00 | 24.41 | C |
| TER | | | | | | | | | | | |
| HETATM | 3208 | P | PO4 | E | 501 | 45.599 | 26.035 | 21.566 | 1.00 | 64.62 | P |
| HETATM | 3209 | O1 | PO4 | E | 501 | 44.644 | 26.754 | 22.504 | 1.00 | 63.84 | O |
| HETATM | 3210 | O2 | PO4 | E | 501 | 44.851 | 25.107 | 20.637 | 1.00 | 63.66 | O |
| HETATM | 3211 | O3 | PO4 | E | 501 | 46.332 | 27.060 | 20.744 | 1.00 | 65.39 | O |
| HETATM | 3212 | O4 | PO4 | E | 501 | 46.617 | 25.222 | 22.329 | 1.00 | 64.30 | O |
| HETATM | 3213 | P | PO4 | E | 503 | 12.139 | 11.754 | 32.621 | 1.00 | 65.14 | P |
| HETATM | 3214 | O1 | PO4 | E | 503 | 11.583 | 11.588 | 34.011 | 1.00 | 66.48 | O |
| HETATM | 3215 | O2 | PO4 | E | 503 | 13.029 | 10.574 | 32.313 | 1.00 | 66.55 | O |
| HETATM | 3216 | O3 | PO4 | E | 503 | 10.997 | 11.840 | 31.636 | 1.00 | 65.51 | O |
| HETATM | 3217 | O4 | PO4 | E | 503 | 12.962 | 13.017 | 32.550 | 1.00 | 66.62 | O |
| HETATM | 3218 | OXT | ACT | E | 504 | 26.790 | 13.748 | 22.887 | 1.00 | 49.23 | O |
| HETATM | 3219 | C | ACT | E | 504 | 26.647 | 12.950 | 23.841 | 1.00 | 49.69 | C |
| HETATM | 3220 | O | ACT | E | 504 | 25.467 | 12.734 | 24.213 | 1.00 | 49.73 | O |
| HETATM | 3221 | CH3 | ACT | E | 504 | 27.823 | 12.314 | 24.522 | 1.00 | 49.34 | C |
| HETATM | 3225 | OXT | ACT | E | 505 | 6.424 | 20.785 | 10.678 | 1.00 | 45.45 | O |
| HETATM | 3226 | C | ACT | E | 505 | 5.899 | 20.163 | 9.730 | 1.00 | 46.04 | C |
| HETATM | 3227 | O | ACT | E | 505 | 5.827 | 18.915 | 9.867 | 1.00 | 46.37 | O |
| HETATM | 3228 | CH3 | ACT | E | 505 | 5.380 | 20.882 | 8.518 | 1.00 | 45.93 | C |
| HETATM | 3232 | OXT | ACT | E | 506 | 20.595 | 16.362 | 10.218 | 1.00 | 48.70 | O |
| HETATM | 3233 | C | ACT | E | 506 | 20.781 | 15.753 | 11.296 | 1.00 | 48.15 | C |
| HETATM | 3234 | O | ACT | E | 506 | 19.779 | 15.624 | 12.037 | 1.00 | 47.36 | O |
| HETATM | 3235 | CH3 | ACT | E | 506 | 22.130 | 15.206 | 11.657 | 1.00 | 48.26 | C |
| TER | | | | | | | | | | | |
| HETATM | 3239 | OH2 | WAT | W | 1 | 32.547 | 16.795 | 15.700 | 1.00 | 28.64 | O |
| HETATM | 3242 | OH2 | WAT | W | 2 | 37.125 | 18.505 | 11.824 | 1.00 | 27.60 | O |
| HETATM | 3245 | OH2 | WAT | W | 3 | 38.737 | 15.512 | 15.931 | 1.00 | 18.70 | O |
| HETATM | 3248 | OH2 | WAT | W | 4 | 22.093 | 30.791 | 29.656 | 1.00 | 28.86 | O |
| HETATM | 3251 | OH2 | WAT | W | 5 | 25.990 | 37.291 | 8.271 | 1.00 | 26.43 | O |
| HETATM | 3254 | OH2 | WAT | W | 6 | 31.562 | 23.648 | 12.209 | 1.00 | 23.20 | O |
| HETATM | 3257 | OH2 | WAT | W | 7 | −8.456 | 36.781 | 14.797 | 1.00 | 32.03 | O |
| HETATM | 3260 | OH2 | WAT | W | 8 | 20.405 | 12.684 | 19.058 | 1.00 | 32.99 | O |
| HETATM | 3263 | OH2 | WAT | W | 9 | 24.583 | 15.052 | 22.792 | 1.00 | 25.21 | O |
| HETATM | 3266 | OH2 | WAT | W | 10 | 19.823 | 32.549 | 22.732 | 1.00 | 20.22 | O |
| HETATM | 3269 | OH2 | WAT | W | 11 | 41.067 | 29.523 | −1.007 | 1.00 | 30.67 | O |
| HETATM | 3272 | OH2A | WAT | W | 12 | 20.802 | 37.112 | 27.477 | 0.50 | 11.80 | O |
| HETATM | 3273 | OH2B | WAT | W | 12 | 22.169 | 37.994 | 28.029 | 0.50 | 21.18 | O |
| HETATM | 3278 | OH2 | WAT | W | 13 | 20.453 | 21.351 | 15.118 | 1.00 | 19.80 | O |
| HETATM | 3281 | OH2 | WAT | W | 15 | 43.126 | 25.927 | 19.070 | 1.00 | 24.06 | O |
| HETATM | 3284 | OH2 | WAT | W | 16 | 24.569 | 15.706 | 29.334 | 1.00 | 24.17 | O |
| HETATM | 3287 | OH2 | WAT | W | 17 | 7.104 | 34.971 | 25.009 | 1.00 | 31.75 | O |
| HETATM | 3290 | OH2 | WAT | W | 18 | 33.431 | 18.728 | 13.717 | 1.00 | 20.05 | O |
| HETATM | 3293 | OH2 | WAT | W | 19 | 3.497 | 36.628 | 24.612 | 1.00 | 31.59 | O |
| HETATM | 3296 | OH2 | WAT | W | 20 | 37.194 | 27.197 | 28.268 | 1.00 | 32.57 | O |
| HETATM | 3299 | OH2 | WAT | W | 21 | 9.205 | 36.745 | 24.095 | 1.00 | 34.40 | O |
| HETATM | 3302 | OH2 | WAT | W | 22 | 28.752 | 15.075 | 16.271 | 1.00 | 23.84 | O |
| HETATM | 3305 | OH2 | WAT | W | 23 | 30.715 | 39.019 | 12.626 | 1.00 | 27.33 | O |
| HETATM | 3308 | OH2 | WAT | W | 24 | 34.902 | 36.850 | 18.316 | 1.00 | 31.31 | O |
| HETATM | 3311 | OH2 | WAT | W | 25 | 40.544 | 20.658 | 22.864 | 1.00 | 20.70 | O |
| HETATM | 3314 | OH2 | WAT | W | 26 | 41.983 | 25.596 | 23.058 | 1.00 | 28.46 | O |
| HETATM | 3317 | OH2 | WAT | W | 27 | 42.007 | 28.244 | 1.012 | 1.00 | 35.33 | O |
| HETATM | 3320 | OH2 | WAT | W | 28 | 8.128 | 44.643 | 16.199 | 1.00 | 38.31 | O |
| HETATM | 3323 | OH2 | WAT | W | 29 | 33.330 | 38.484 | 11.675 | 1.00 | 19.72 | O |
| HETATM | 3326 | OH2 | WAT | W | 30 | 15.624 | 27.254 | 30.952 | 1.00 | 30.46 | O |
| HETATM | 3329 | OH2 | WAT | W | 31 | 15.577 | 30.133 | 4.279 | 1.00 | 41.69 | O |
| HETATM | 3332 | OH2 | WAT | W | 32 | 34.334 | 37.525 | 21.968 | 1.00 | 31.93 | O |
| HETATM | 3335 | OH2 | WAT | W | 33 | 24.151 | 35.466 | 29.014 | 1.00 | 31.48 | O |
| HETATM | 3338 | OH2 | WAT | W | 34 | 17.575 | 22.913 | 10.433 | 1.00 | 32.25 | O |
| HETATM | 3341 | OH2 | WAT | W | 35 | 35.852 | 34.072 | 6.593 | 1.00 | 29.81 | O |
| HETATM | 3344 | OH2 | WAT | W | 36 | 42.234 | 36.226 | 24.504 | 1.00 | 35.93 | O |
| HETATM | 3347 | OH2 | WAT | W | 37 | 16.297 | 39.267 | 20.892 | 1.00 | 28.99 | O |
| HETATM | 3350 | OH2 | WAT | W | 38 | −2.843 | 27.914 | 20.117 | 1.00 | 29.68 | O |
| HETATM | 3353 | OH2 | WAT | W | 39 | 18.534 | 15.396 | 18.811 | 1.00 | 39.63 | O |
| HETATM | 3356 | OH2 | WAT | W | 40 | 24.155 | 26.497 | 7.069 | 1.00 | 35.89 | O |
| HETATM | 3359 | OH2 | WAT | W | 41 | 33.650 | 16.970 | 27.999 | 1.00 | 33.65 | O |
| HETATM | 3362 | OH2 | WAT | W | 42 | −0.388 | 39.702 | 19.672 | 1.00 | 39.66 | O |
| HETATM | 3365 | OH2 | WAT | W | 43 | 24.302 | 24.792 | 4.841 | 1.00 | 43.59 | O |
| HETATM | 3368 | OH2 | WAT | W | 44 | 47.548 | 29.966 | 18.465 | 1.00 | 23.65 | O |
| HETATM | 3371 | OH2 | WAT | W | 45 | 11.835 | 38.833 | 5.233 | 1.00 | 32.10 | O |
| HETATM | 3374 | OH2 | WAT | W | 46 | 30.397 | 25.184 | −0.264 | 1.00 | 33.96 | O |
| HETATM | 3377 | OH2 | WAT | W | 47 | 13.764 | 35.420 | 24.711 | 1.00 | 33.61 | O |
| HETATM | 3380 | OH2 | WAT | W | 48 | 29.173 | 36.078 | 2.463 | 1.00 | 30.30 | O |
| HETATM | 3383 | OH2 | WAT | W | 49 | 16.309 | 13.865 | 18.825 | 1.00 | 36.03 | O |
| HETATM | 3386 | OH2 | WAT | W | 50 | 35.208 | 16.762 | 25.738 | 1.00 | 32.78 | O |
| HETATM | 3389 | OH2 | WAT | W | 51 | 31.482 | 34.209 | 30.209 | 1.00 | 37.52 | O |
| HETATM | 3392 | OH2 | WAT | W | 52 | 25.824 | 27.278 | 34.713 | 1.00 | 42.43 | O |
| HETATM | 3395 | OH2 | WAT | W | 53 | 9.626 | 39.517 | 20.343 | 1.00 | 38.93 | O |
| HETATM | 3398 | OH2 | WAT | W | 54 | 14.848 | 11.406 | 22.943 | 1.00 | 41.31 | O |
| HETATM | 3401 | OH2 | WAT | W | 55 | 39.442 | 15.246 | 26.378 | 1.00 | 35.42 | O |
| HETATM | 3404 | OH2 | WAT | W | 56 | 14.322 | 10.348 | 26.707 | 1.00 | 50.40 | O |
| HETATM | 3407 | OH2 | WAT | W | 57 | −2.182 | 19.203 | 11.582 | 1.00 | 41.07 | O |
| HETATM | 3410 | OH2 | WAT | W | 58 | 9.101 | 34.455 | 20.025 | 1.00 | 29.42 | O |
| HETATM | 3413 | OH2 | WAT | W | 59 | 7.345 | 28.907 | 5.837 | 1.00 | 45.92 | O |
| HETATM | 3416 | OH2 | WAT | W | 60 | −6.006 | 29.893 | 7.513 | 1.00 | 39.87 | O |
| HETATM | 3419 | OH2 | WAT | W | 61 | −5.430 | 40.216 | 20.373 | 1.00 | 41.40 | O |
| HETATM | 3422 | OH2 | WAT | W | 62 | 12.641 | 36.612 | 3.700 | 1.00 | 41.68 | O |
| HETATM | 3425 | OH2 | WAT | W | 63 | 7.957 | 14.873 | 20.330 | 1.00 | 45.20 | O |
| HETATM | 3428 | OH2 | WAT | W | 64 | 5.704 | 22.797 | 31.109 | 1.00 | 40.02 | O |
| HETATM | 3431 | OH2 | WAT | W | 65 | 32.011 | 20.629 | 34.409 | 1.00 | 63.02 | O |
| HETATM | 3434 | OH2 | WAT | W | 66 | 22.257 | 28.953 | 33.833 | 1.00 | 42.44 | O |
| HETATM | 3437 | OH2A | WAT | W | 67 | 19.233 | 33.354 | 34.685 | 0.50 | 17.99 | O |
| HETATM | 3438 | OH2B | WAT | W | 67 | 19.831 | 31.531 | 35.750 | 0.50 | 36.39 | O |
| HETATM | 3443 | OH2 | WAT | W | 68 | 44.018 | 31.750 | 27.329 | 1.00 | 40.52 | O |
| HETATM | 3446 | OH2 | WAT | W | 69 | 22.768 | 24.339 | 10.863 | 1.00 | 32.55 | O |
| HETATM | 3449 | OH2 | WAT | W | 70 | 19.671 | 22.446 | 12.717 | 1.00 | 29.10 | O |
| HETATM | 3452 | OH2 | WAT | W | 71 | 35.795 | 34.909 | 16.247 | 1.00 | 34.94 | O |
| HETATM | 3455 | OH2 | WAT | W | 72 | 48.886 | 28.297 | 8.973 | 1.00 | 40.07 | O |
| HETATM | 3458 | OH2 | WAT | W | 73 | 41.485 | 22.658 | 7.786 | 1.00 | 26.14 | O |
| HETATM | 3461 | OH2 | WAT | W | 74 | 44.340 | 18.734 | 15.139 | 1.00 | 34.73 | O |
| HETATM | 3464 | OH2 | WAT | W | 75 | 40.077 | 26.312 | 32.109 | 1.00 | 49.52 | O |
| HETATM | 3467 | OH2 | WAT | W | 76 | 27.330 | 39.414 | 18.719 | 1.00 | 32.80 | O |
| HETATM | 3470 | OH2 | WAT | W | 77 | 24.554 | 9.528 | 25.075 | 1.00 | 38.00 | O |
| HETATM | 3473 | OH2 | WAT | W | 78 | 3.393 | 17.850 | 21.209 | 1.00 | 50.66 | O |
| HETATM | 3476 | OH2 | WAT | W | 79 | −1.363 | 24.295 | 24.042 | 1.00 | 38.38 | O |
| HETATM | 3479 | OH2 | WAT | W | 80 | 17.649 | 15.226 | 11.055 | 1.00 | 55.54 | O |
| HETATM | 3482 | OH2 | WAT | W | 81 | 13.380 | 38.200 | 23.209 | 1.00 | 39.19 | O |
| HETATM | 3485 | OH2 | WAT | W | 82 | 13.715 | 43.032 | 16.727 | 1.00 | 33.87 | O |
| HETATM | 3488 | OH2 | WAT | W | 83 | 7.963 | 43.838 | 21.440 | 1.00 | 49.40 | O |
| HETATM | 3491 | OH2 | WAT | W | 85 | −0.316 | 25.598 | 25.994 | 1.00 | 35.04 | O |
| HETATM | 3494 | OH2 | WAT | W | 86 | 9.936 | 22.321 | 11.241 | 1.00 | 36.37 | O |
| HETATM | 3497 | OH2 | WAT | W | 87 | 13.877 | 35.043 | 28.710 | 1.00 | 54.71 | O |
| HETATM | 3500 | OH2A | WAT | W | 88 | 15.825 | 38.135 | 24.245 | 0.50 | 27.89 | O |
| HETATM | 3501 | OH2B | WAT | W | 88 | 17.812 | 38.640 | 24.382 | 0.50 | 21.72 | O |
| HETATM | 3506 | OH2 | WAT | W | 89 | 25.771 | 38.079 | 22.141 | 1.00 | 44.71 | O |
| HETATM | 3509 | OH2 | WAT | W | 90 | 35.859 | 35.896 | 26.795 | 1.00 | 36.76 | O |
| HETATM | 3512 | OH2 | WAT | W | 91 | 44.041 | 26.955 | 16.540 | 1.00 | 25.08 | O |
| HETATM | 3515 | OH2 | WAT | W | 92 | 49.374 | 23.918 | 21.559 | 1.00 | 46.82 | O |
| HETATM | 3518 | OH2 | WAT | W | 93 | 46.632 | 30.868 | 26.731 | 1.00 | 32.84 | O |
| HETATM | 3521 | OH2 | WAT | W | 94 | 26.246 | 18.459 | 13.078 | 1.00 | 34.81 | O |
| HETATM | 3524 | OH2 | WAT | W | 95 | 30.959 | 21.906 | 10.078 | 1.00 | 39.46 | O |
| HETATM | 3527 | OH2 | WAT | W | 96 | 34.332 | 18.635 | 4.090 | 1.00 | 41.96 | O |
| HETATM | 3530 | OH2 | WAT | W | 97 | 21.050 | 24.858 | 4.221 | 1.00 | 39.10 | O |
| HETATM | 3533 | OH2 | WAT | W | 98 | 18.266 | 29.895 | 3.499 | 1.00 | 47.85 | O |
| HETATM | 3536 | OH2 | WAT | W | 99 | 41.833 | 22.970 | 23.325 | 1.00 | 43.50 | O |
| HETATM | 3539 | OH2 | WAT | W | 100 | 18.357 | 11.671 | 22.171 | 1.00 | 40.08 | O |
| HETATM | 3542 | OH2 | WAT | W | 101 | 25.473 | 12.177 | 14.235 | 1.00 | 32.22 | O |
| HETATM | 3545 | OH2 | WAT | W | 102 | 0.077 | 33.054 | 6.317 | 1.00 | 43.00 | O |
| HETATM | 3548 | OH2 | WAT | W | 103 | −7.330 | 40.478 | 18.599 | 1.00 | 56.76 | O |
| HETATM | 3551 | OH2 | WAT | W | 104 | 20.411 | 41.455 | 19.742 | 1.00 | 53.01 | O |
| HETATM | 3554 | OH2 | WAT | W | 105 | 46.712 | 25.597 | 24.694 | 1.00 | 39.46 | O |
| HETATM | 3557 | OH2 | WAT | W | 106 | 40.001 | 37.186 | 21.842 | 1.00 | 36.50 | O |
| HETATM | 3560 | OH2 | WAT | W | 107 | 41.180 | 35.937 | 17.194 | 1.00 | 41.52 | O |
| HETATM | 3563 | OH2 | WAT | W | 108 | 29.536 | 36.648 | 27.528 | 1.00 | 34.31 | O |
| HETATM | 3566 | OH2 | WAT | W | 109 | 33.496 | 35.655 | 4.170 | 1.00 | 35.21 | O |
| HETATM | 3569 | OH2 | WAT | W | 110 | 38.363 | 16.291 | 9.765 | 1.00 | 43.85 | O |
| HETATM | 3572 | OH2 | WAT | W | 111 | 11.827 | 23.493 | 35.283 | 1.00 | 34.24 | O |
| HETATM | 3575 | OH2 | WAT | W | 112 | 9.679 | 24.493 | 35.404 | 1.00 | 30.98 | O |
| TER | | | | | | | | | | | |
| END | | | | | | | | | | | |

1. A method for identifying a potential inhibitor of HIV-1 protease, the method comprising:
generating a three-dimensional structural model of a molecule or molecular complex comprising an HIV-1 protease active site; and
employing the three-dimensional structural model to design or select a potential inhibitor, wherein the potential inhibitor forms at least one hydrogen bond with the backbone of amino acids 50 and 50′ of the HIV-1 protease via an acyclic group, a sulfonyl group, or a selenonyl group, without an intervening water molecule.

2. The method of claim 1, wherein the potential inhibitor is a small organic molecule inhibitor that forms at least one hydrogen bond with the backbone of amino acids 50 and 50′ of the HIV-1 protease via an acyclic group without an intervening water molecule.

3. The method of claim 1, wherein the potential inhibitor further forms at least one hydrogen bond with the conserved side chain of at least one of amino acids Asp25 and Asp25′ of the HIV-1 protease.

4. The method of claim 3, wherein the hydrogen bond with the side chain is formed by a primary hydroxyl, thiol, or amino group on the potential inhibitor.

5. The method of claim 3, wherein the hydrogen bond is a bifurcated hydrogen bond.

6. The method of claim 1, wherein the potential inhibitor does not hydrogen bond with amino acid 27 of the HIV-1 protease.

7. The method of claim 1, wherein the potential inhibitor forms at least one hydrogen bond with the backbone of amino acids 48 or 28 of the HIV-1 protease.

8. The method of claim 1, wherein the potential inhibitor forms at least one hydrogen bond with the conserved side chain of amino acid Asp29 of the HIV-1 protease.

9. The method of claim 1, wherein the potential inhibitor has the following interactions with the HIV-1 protease:
(a) hydrogen bonding with the backbone of amino acids 50 and 50′ of the HIV-1 protease, without an intervening water molecule; and
(b) direct hydrogen bonding with the conserved side chain of amino acid Asp25 and Asp25′ of the HIV-1 protease;
and two or more of the following:
(c) no hydrogen bonding interaction with any atom of amino acid 27 of the HIV-1 protease;
(d) direct hydrogen bonding with the backbone nitrogen of amino acids 48 and 28 of the HIV-1 protease; and
(e) direct hydrogen bonding with at least one of the oxygen atoms of the conserved side chain of amino acid Asp29 of the HIV-1 protease.

10. The method of claim 9, wherein the potential inhibitor has all of interactions (a)-(e) with the HIV-1 protease.

11. The method of claim 9, wherein the potential inhibitor additionally has one of the following interactions with the HIV-1 protease:
(f) direct hydrogen bonding with the backbone, side chains, or both, of one or more of amino acids 29′, 30′, and 48′ of the HIV-1 protease; or
(g) indirect hydrogen bonding with the backbone, side chains, or both, of one or more of amino acids 29′, 30′, and 48′ of the HIV-1 protease.

12. The method of claim 1, wherein generating a three-dimensional structural model comprises using at least the atomic coordinates of HIV-1 protease amino acids 24-30, 24′-30′, 47-53, 47′-53′, 84 and 84′, and optionally amino acids 82 and 82′, according to Table 2± a root mean square deviation from the backbone atoms of said amino acids of not more than 1.5 Å.

13. The method of claim 1, wherein generating a three-dimensional structural model comprises using the atomic coordinates of HIV-1 protease according to Table 2± a root mean square deviation from the backbone atoms of said amino acids of not more than 1.5 Å.

14. The method of claim 1, further comprising:
synthesizing or obtaining the potential inhibitor;
contacting the potential inhibitor with a sample comprising an HIV-1 protease; and
determining the ability of the potential inhibitor to bind to and/or inhibit protease activity of the HIV-1 protease.

15. The method of claim 14, further comprising subjecting the potential inhibitor to cross-resistance profiling.

16. The method of claim 14, further comprising determining the binding affinity of the potential inhibitor for the HIV-1 protease.

17. The method of claim 1, wherein employing the three-dimensional structural model to design or select a potential inhibitor comprises:
computationally performing a fitting operation between the computer model of the protease active site and the computer model of the potential inhibitor, and
evaluating the results of the fitting operation to determine the ability of the potential inhibitor to interact with the protease active site, and/or to characterize the interaction of the potential inhibitor with the active site.

18. The method of claim 17, wherein the fitting operation comprises determining an energy minima configuration of computer model of the three-dimensional structure of the potential inhibitor in the computer model of the three-dimensional structure of the protease active site.

19. The method of claim 1 wherein the potential inhibitor is: (i) computationally assembled molecular fragments; (ii) selected from a small molecule database; or (iii) computationally created by de novo molecule design.

20. A crystal comprising HIV-1 protease complexed with inhibitor P867883, having space group P2₁2₁2₁.

21. The crystal of claim 20, having the following properties:

| | |
|---|---|
| a (Å) | 51.11 |
| b (Å) | 58.10 |
| c (Å) | 61.60 |

22. The method of claim 1, further comprising providing a crystal comprising HIV-1 protease complexed with inhibitor P867883, having space group P2₁2₁2₁.

23. The method of claim 1, wherein employing the three-dimensional structural model to design or select a potential inhibitor comprises:
providing a three-dimensional model of the potential inhibitor, and
employing computational means to perform a fitting operation between the model of the potential inhibitor and the model of the HIV-1 protease active site to provide an energy minimized configuration of the potential inhibitor in the active site; and
evaluating the results of the fitting operation to design or select the potential inhibitor.

24. The method of claim 1, wherein the potential inhibitor forms at least one hydrogen bond with the backbone nitrogen of amino acids 50 and 50′ of the HIV-1 protease.

25. The method of claim 3, wherein the potential inhibitor forms direct hydrogen bonding with the side chain oxygen atoms of amino acid Asp25 and Asp25′ of the HIV-1 protease.

26. The method of claim 1, wherein the potential inhibitor is selected by assembly of molecular fragments.

27. The method of claim 1, wherein the potential inhibitor is selected by de novo ligand design.

28. The method of claim 19, wherein the potential inhibitor is selected from a database of compounds.

29. The method of claim 9, wherein the potential inhibitor forms direct hydrogen bonding with the side chain oxygen atoms of amino acid Asp25 and Asp25′ of the HIV-1 protease.
